SEQUENCE LISTING (19 sequences)

SEQ ID NO: 1 - DNA, 41 bp, synthetic construct
  source 1..41: mol_type = other DNA
  modified_base 1: mod_base = OTHER; note = PHOSPHORYLATION
acaaaggcagccacgcactccttccctgaaggccggaatct

SEQ ID NO: 2 - DNA, 41 bp, synthetic construct
  source 1..41: mol_type = other DNA
  modified_base 1: mod_base = OTHER; note = PHOSPHORYLATION
gctgccgccactagccatcttactgctgaggactcttcgct

SEQ ID NO: 3 - DNA, 40 bp, synthetic construct
  source 1..40: mol_type = other DNA
  modified_base 1: mod_base = OTHER; note = PHOSPHORYLATION
gattccggccttgtggttggtgagggtcatctcgctggag

SEQ ID NO: 4 - DNA, 40 bp, synthetic construct
  source 1..40: mol_type = other DNA
  modified_base 1: mod_base = OTHER; note = PHOSPHORYLATION
gcgaagagtcctggagtgccgccaatgtatgcgagggtga

SEQ ID NO: 5 - DNA, 71 bp, synthetic construct
  source 1..71: mol_type = other DNA
  modified_base 1: mod_base = OTHER; note = PHOSPHORYLATION
gcgcgcgttttttttgcttgcgtctcctgccagccatatccggtctacgtgatccttttttttcgcgcgct

SEQ ID NO: 6 - DNA, 83 bp, synthetic construct
  source 1..83: mol_type = other DNA
  modified_base 1: mod_base = OTHER; note = PHOSPHORYLATION
gcgcgcgttttttttttttttgcttgcgtctcctgccagccatatccggtctacgtgatccttttttttttttttcgcgcgct

SEQ ID NO: 7 - DNA, 62 bp, synthetic construct
  source 1..62: mol_type = other DNA
  modified_base 1: mod_base = OTHER; note = PHOSPHORYLATION
ggatcacgtagattttgcttgcgtctcctgccagccatatccggtttttctacgtgattcct

SEQ ID NO: 8 - DNA, 81 bp, synthetic construct
  source 1..81: mol_type = other DNA
  modified_base 1: mod_base = OTHER; note = PHOSPHORYLATION
gcgaagagtcctggagtgccgccaatgtatgcgagggtgagctgccgccactagccatcttactgctgaggactcttcgct

SEQ ID NO: 9 - DNA, 93 bp, synthetic construct
  source 1..93: mol_type = other DNA
  modified_base 1: mod_base = OTHER; note = PHOSPHORYLATION
gcgaagagtcctttttttggagtgccgccaatgtatgcgagggtgagctgccgccactagccatcttactgctgttttttaggactcttcgct

SEQ ID NO: 10 - DNA, 100 bp, synthetic construct
  source 1..100: mol_type = other DNA
  modified_base 1: mod_base = OTHER; note = PHOSPHORYLATION
gcgaagagtcctttttttggagtgccgccaatgtatgcgagggtgatttttttgctgccgccactagccatcttactgctgttttttaggactcttcgct

SEQ ID NO: 11 - DNA, 81 bp, synthetic construct
  source 1..81: mol_type = other DNA
  modified_base 1: mod_base = OTHER; note = PHOSPHORYLATION
gattccggccttgtggttggtgagggtcatctcgctggagacaaaggcagccacgcactccttccctgaaggccggaatct

SEQ ID NO: 12 - DNA, 93 bp, synthetic construct
  source 1..93: mol_type = other DNA
  modified_base 1: mod_base = OTHER; note = PHOSPHORYLATION
gattccggccttttttttgtggttggtgagggtcatctcgctggagacaaaggcagccacgcactccttccctgttttttaaggccggaatct

SEQ ID NO: 13 - DNA, 100 bp, synthetic construct
  source 1..100: mol_type = other DNA
  modified_base 1: mod_base = OTHER; note = PHOSPHORYLATION
gattccggccttttttttgtggttggtgagggtcatctcgctggagtttttttacaaaggcagccacgcactccttccctgttttttaaggccggaatct

SEQ ID NO: 14 - DNA, 61 bp, synthetic construct
  source 1..61: mol_type = other DNA
  modified_base 1: mod_base = OTHER; note = PHOSPHORYLATION
ggatcacgtagattttgcttgcgtctcctgccagccatatccggtttttctacgtgatcct

SEQ ID NO: 15 - DNA, 77 bp, synthetic construct
  source 1..77: mol_type = other DNA
  modified_base 1: mod_base = OTHER; note = PHOSPHORYLATION
ggatcacgtagattttttttttttgcttgcgtctcctgccagccatatccggtttttttttttttctacgtgatcct

SEQ ID NO: 16 - DNA, 101 bp, synthetic construct
  source 1..101: mol_type = other DNA
  modified_base 1: mod_base = OTHER; note = PHOSPHORYLATION
ggatcacgtagattttttttttttttttttttttttgcttgcgtctcctgccagccatatccggtttttttttttttttttttttttttctacgtgatcct

SEQ ID NO: 17 - DNA, 103 bp, synthetic construct
  source 1..103: mol_type = other DNA
  modified_base 1: mod_base = OTHER; note = PHOSPHORYLATION
ggatcacgtagattttttttttttttttttttttttttttttttttttttttgcttgcgtctcctgccagccatatccggtttttttttttctacgtgatcct

SEQ ID NO: 18 - DNA, 67 bp, synthetic construct
  source 1..67: mol_type = other DNA
  modified_base 1: mod_base = OTHER; note = PHOSPHORYLATION
ggatcacgtagattttagatctgcttgcgtctcctgccagccatatccggtttttctacgtgatcct

SEQ ID NO: 19 - DNA, 82 bp, synthetic construct
  source 1..82: mol_type = other DNA
  modified_base 1: mod_base = OTHER; note = PHOSPHORYLATION
ggatcacgtagattttttttttttagatctgcttgcgtctcctgccagccatatccggttttttttttttctacgtgatcct

(19) United States
(12) Patent Application Publication
(10) Pub. No.: US 2024/0271208 A1
(43) Pub. Date: Aug. 15, 2024

(54) METHODS FOR DIFFERENTIATING MODIFIED NUCLEOBASES

(71) Applicant: Singular Genomics Systems, Inc., San Diego, CA (US)

(72) Inventor: Eli N. Glezer, Del Mar, CA (US)

(21) Appl. No.: 18/592,127

(22) Filed: Feb. 29, 2024

Related U.S. Application Data
(63) Continuation of application No. PCT/US2022/075688, filed on Aug. 30, 2022.
(60) Provisional application No. 63/240,670, filed on Sep. 3, 2021.
(60) Provisional application No. 63/311,571, filed on Feb. 18, 2022.

(51) Int. Cl. C12Q 1/6874; C12Q 1/34; C12Q 1/48

(52) U.S. Cl. CPC C12Q 1/6874; C12Q 1/34; C12Q 1/48; C12Q 2600/166

(57) ABSTRACT

Disclosed herein, inter alia, are compositions, methods, and kits useful for detecting nucleobase modifications on one or both strands of a double-stranded nucleic acid fragment.

### CROSS-REFERENCES TO RELATED APPLICATIONS

This application claims the benefit of U.S. Provisional Application No. 63/240,670, filed Sep. 3, 2021, and U.S. Provisional Application No. 63/311,571, filed Feb. 18, 2022, each of which are incorporated herein by reference in their entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 051385-557001WO_ST26.xml, created Aug. 12, 2022, 29,074 bytes, machine format IBM-PC, MS Windows operating system, is hereby incorporated by reference.

### BACKGROUND

Liquid biopsies show promise for early detection and therapeutic interventions for metastatic cancers. Repeated biopsies of metastatic lesions are invasive and, depending on the location of the cancerous tissue, difficult to access. In contrast, monitoring of biological samples (e.g., blood, plasma, or other bodily fluids) is minimally invasive and can identify tumor evolution and tumor subtype switches, which may then lead to the selection of appropriate therapies. For example, liquid biopsies analyzing the circulating tumor DNA (ctDNA) found in blood, pancreatic cysts, Pap smears, and saliva have been performed. Epigenetic information, such as biomolecule methylation, and/or -additional protein biomarkers combined with cfDNA and ctDNA analyses, is useful in determining the origin of cancer at an early stage. Biomolecule methylation, such as DNA methylation, is widespread and plays a critical role in the regulation of gene expression in development, differentiation, and disease. Disclosed herein, inter alia, are solutions to these and other problems in the art.

### BRIEF SUMMARY

In an aspect is provided compositions, kits, and methods for differentiating modifications to nucleobases (e.g., discerning chemical modifications to cytosine nucleobases) within double-stranded nucleic acids.

In an aspect is provided a method of sequencing a nucleic acid molecule, wherein the nucleic acid molecule includes, from 5′ to 3′, a first strand, a first primer binding sequence, a second strand including a cytosine nucleobase, and a second primer binding sequence, wherein the second strand is complementary to the first strand, the method including: (a) annealing a blocking primer to the first primer binding sequence of the nucleic acid molecule and extending the blocking primer with a polymerase to form a blocking strand hybridized to the first strand; (b) converting the cytosine nucleobase of the second strand to a uracil nucleobase, or uracil nucleobase analog; and (c) sequencing the second strand to generate a sequencing read.

In an aspect is provided a method of generating a double-stranded nucleic acid including a cytosine mismatch, the method including: (a) ligating a first hairpin adapter to a first end of the double-stranded nucleic acid molecule, and ligating a second adapter to a second end of the double-stranded nucleic acid, thereby forming a nucleic acid template, wherein the double-stranded nucleic acid includes a first strand hybridized to a second strand, and wherein the second strand includes a cytosine nucleobase; (b) annealing a blocking primer to a sequence of the nucleic acid template and extending the blocking primer, thereby generating a blocking strand hybridized to the first strand of the double-stranded nucleic acid, and displacing the second strand of the double-stranded nucleic acid; (c) converting the cytosine nucleobase of the displaced second strand to a uracil nucleobase, or uracil nucleobase analog, to form a converted second strand; (d) removing the blocking strand and hybridizing the first strand to the converted second strand, thereby forming the double-stranded nucleic acid including a cytosine mismatch.

In an aspect is provided a polynucleotide including, from 5′ to 3′, a first primer binding sequence, a first template single-stranded nucleic acid sequence including an unmodified cytosine nucleobase, an adapter, a second template single-stranded nucleic acid, and a second primer binding sequence, wherein the first template single-stranded nucleic acid sequence is hybridized to a blocking nucleic acid sequence; and the second template single-stranded nucleic acid comprises a modified cytosine nucleobase.

In an aspect is provided a polynucleotide including, from 5′ to 3′, a first primer binding sequence, a first template single-stranded nucleic acid sequence, an adapter, a second template single-stranded nucleic acid, and a second primer binding sequence, wherein the second template single-stranded nucleic acid sequence is hybridized to a blocking nucleic acid sequence; and the first template single-stranded nucleic acid includes a modified cytosine nucleobase.

### BRIEF DESCRIPTION OF THE DRAWINGS

FIGS. 1A-1B. FIG. 1A shows an embodiment of an adapter-target-adapter template including a double stranded nucleic acid of interest annealed to a Y-adapter and a hairpin adapter. FIG. 1B shows an embodiment of an adapter-target-adapter template where a double stranded nucleic acid of interest is annealed to a first hairpin adapter (hairpin adapter 1) and a second, non-identical, hairpin adapter (hairpin adapter 2). Primer binding sites (i.e., sequences having complementarity to a specific primer) are identified as P1, P2′, and P3 indicating unique primer binding sites, or complements thereof.

FIGS. 2A-2D show embodiments of adapters. FIG. 2A shows an embodiment of a Y adapter including (i) a first strand having a 5′-arm and a 3′-portion, and (ii) a second strand having a 5′-portion and a 3′-arm, wherein the 3′-portion of the first strand is substantially complementary to the 5′-portion of the second strand, and the 5′-arm of the first strand is not substantially complementary to the 3′-arm of the second strand. In this embodiment, the complementary portions (i.e., duplex regions) of the Y adapter include a melting temperature (Tm) of about 40-45° C. and a length of about 10 to 15 nucleotides. In embodiments, the complementary portions (i.e., duplex regions) of the Y adapter include a Tm (melting temperature) of about 35-45° C. or 30-45° C. and a length of about 12 bases. FIG. 2B shows an embodiment of a hairpin adapter including a 5′-end, a 5′ portion, a loop, a 3′ portion and a 3′-end. In this embodiment, a duplex region of the hairpin adapter includes a Tm (melting temperature) of about 40-45° C. and a length of about 10-16 bases. In embodiments, the duplex region of the adapter includes a Tm (melting temperature) of about 35-45° C. or 30-45° C. and a length of about 12 bases. FIG. 2C illustrates an embodiment of a hairpin adapter, which includes a double stranded (stem) region and a loop region. Within the loop region is a priming site (P3) and optionally a unique molecular identifier (UMI). FIG. 2D illustrates the adapters may include different duplex ends. For example, the double-stranded region of a Y adapter (alternatively referred to as a forked adapter) may be blunt-ended (top), have a 3′ overhang (middle), or a 5′ overhang (bottom). On the right are embodiments of hairpin adapters, each including a 5′-end and a 3′-end. In some embodiments, a hairpin adapter includes a double stranded portion (a double-stranded “stem” region) and a loop, where 5′P refers to a phosphorylated 5′ end. A double-stranded stem region of a hairpin adapter may be blunt-ended (top), it may have a 5′ overhang (middle), or a 3′ overhang (bottom). An overhang may include a single nucleotide or more than one nucleotide.

FIGS. 3A-3B illustrate embodiments for conversion of nucleobases. FIG. 3A illustrates bisulfite conversion, which converts a cytosine nucleobase to a uracil nucleobase (top), however modified cytosine nucleobases (e.g., 5-methylcytosine (5mC) or 5-hydroxymethyl cytosine (5hmC)) are not susceptible to bisulfite conversion methods (bottom). FIG. 3B illustrates an alternate conversion approach, which combines a first enzymatic conversion of a modified cytosine nucleobase (e.g., 5-methylcytosine (5mC) or 5-hydroxymethyl cytosine (5hmC)) to an intermediate nucleobase, 5-carboxylcytosine (5caC), followed by a subsequent conversion to a uracil nucleobase analog, dihydrouridine (DHU).

FIGS. 4A-4C provides an overview of different single-stranded conversion approaches. FIG. 4A illustrates a portion of a single-stranded polynucleotide sequence that contains modified cytosines and non-modified cytosines, e.g., 5′-[A][T][^{5hm}C][A][C][^{5m}C]-3′-, where [^{5hm}C] refers to 5-hydroxymethyl cytosine and [5mC] refers to 5-methylcytosine. Following a first chemical conversion step (e.g., bisulfite conversion) the non-modified cytosine nucleobases are converted to uracil nucleobases. When this single-stranded polynucleotide is subjected to standard amplification methods (e.g., PCR), the resulting polynucleotide includes thymidine nucleobases in the positions where the non-modified cytosine nucleobases were in the original polynucleotide sequence. FIG. 4B depicts a combination of enzymatic and chemical conversion protocols (e.g., TAPS (TET-assisted pyridine borane sequencing)) that convert the modified cytosine nucleobases. Briefly, a portion of a single-stranded polynucleotide sequence that contains modified cytosines and non-modified cytosines, e.g., 5′[A][T][^{5hm}C][A][C][^{5m}C]-3′-, is subjected to a first enzymatic conversion (e.g., a TET (ten-eleven translocation methylcytosine dioxygenase) enzyme conversion), which converts the modified cytosine nucleobases to an intermediate nucleobase, 5-carboxylcytosine (5caC). A chemical conversion (e.g., contacting the polynucleotide with borane derivatives (e.g., pyridine borane and 2-picoline borane), which converts the 5caC nucleobases to a uracil nucleobase analog, dihydrouridine (DHU). Following standard amplification protocols (e.g., PCR), the DHU nucleobases are amplified as thymidine nucleobases and the non-modified cytosine nucleobases are amplified as cytosines. FIG. 4C depicts an alternate enzymatic approach. Similar to the approach described in the beginning of FIG. 4B, a portion of a single-stranded polynucleotide sequence that contains modified cytosines and non-modified cytosines, e.g., 5′-[A][T][^{5hm}C][A][C][^{5m}C]-3′-, is subjected to a first enzymatic conversion (e.g., a TET enzyme conversion), which converts the modified cytosine nucleobases to an intermediate nucleobase, 5-carboxylcytosine (5caC). A second enzymatic conversion (e.g., APOBEC enzyme conversion) converts the non-modified cytosine nucleobases to uracil nucleobases. When this single-stranded polynucleotide is subjected to standard amplification methods (e.g., PCR), the resulting polynucleotide includes thymidine nucleobases in the positions where the non-modified cytosine nucleobases were in the original polynucleotide sequence and the modified cytosine nucleobases are amplified as non-modified cytosines.

FIGS. 5A-5D illustrates an embodiment of the method described herein. FIG. 5A illustrates a nucleic acid template containing a first Y adapter, a double stranded nucleic acid, and a hairpin adapter. The double-stranded nucleic acid includes modified cytosine nucleobases, illustrated as triangles on both strands of the nucleic acid. In this embodiment, a primer anneals to the loop region of the hairpin and is extended by a polymerase (depicted as the squishy cloud) to generate a blocking strand. The blocking strand is hybridized to one of the two strands of the double-stranded nucleic acid, whereas the other strand is rendered single-stranded (FIG. 5B). Now that one of the two strands from the original dsDNA template is liberated, a conversion technique may be applied as known in the art and described herein. For example, the enzymatic and chemical conversion method depicted in FIG. 4B may be applied, which converts the modified cytosine nucleobases (depicted as triangles) to uracil nucleobase analogs (depicted as squares) as shown in FIG. 5C. Once the blocking strand is removed, the template nucleic acid may reanneal as depicted in FIG. 5D, providing a template nucleic acid with asymmetric modifications (i.e., one strand contains modified cytosine nucleobases and the other strand contains converted cytosines (e.g., uracil nucleobase analogs)).

FIGS. 6A-6C presents an embodiment of an amplification method for linked methylation detection of a cytosine-converted adapter-target-adapter construct. FIG. 6A shows a cytosine-converted Y-template-hairpin construct (generated as described in FIGS. 5A-5D) hybridizing to an immobilized P2 primer. In the presence of a polymerase, a copy of the original template is made; this copy then hybridizes to an immobilized P1 primer. The uracil or uracil nucleobases (depicted as squares) are copied as an adenine, while the modified cytosine nucleobases (depicted as triangles) are copied as guanines. FIG. 6B depicts annealing, extending, denaturing, re-annealing, and extending steps common to one embodiment of an amplification method for a cytosine-converted construct. FIG. 6C shows an amplified, cytosine-converted Y-template-hairpin construct hybridizing to an immobilized P2 primer. As this was amplified prior to hybridization, the uracil nucleobase analog has now been replaced with a thymine nucleobase. In the presence of a polymerase, a copy of the original template is made; this copy then hybridizes to an immobilized P1 primer as shown in FIG. 6C.

FIGS. 7A-7B presents an embodiment of a linked duplex sequencing process; the gray ellipse represents a polymerase. FIG. 7A shows a process where a template bound to an immobilized P1 is optionally cleaved (i.e., the cleavable site is indicated as ‘X’) and removed. The P2-anchored strands are terminated using a suitable technique (e.g., depicted is a dideoxynucleotide (dd), however any suitable terminating process is contemplated herein). FIG. 7B shows sequencing up with a strand displacing polymerase (left), following by priming at the P3 priming site and sequencing down with a strand displacing polymerase (right).

### DETAILED DESCRIPTION

### I. Definitions

All patents, patent applications, articles and publications mentioned herein, both supra and infra, are hereby expressly incorporated herein by reference in their entireties. The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices and materials similar or equivalent to those described herein can be used in the practice of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides (e.g., Watson-Crick base pairing). As described herein and commonly known in the art the complementary (matching) nucleotide of adenosine is thymidine and the complementary (matching) nucleotide of guanosine is cytosine. Thus, a complement may include a sequence of nucleotides that base paired with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As used herein, the term “complementary” or “substantially complementary” refers to the hybridization, base pairing, or the formation of a duplex between nucleotides or nucleic acids. When referring to a double-stranded polynucleotide including a first strand hybridized to a second strand, it is to be understood that each of the terms “first strand” and “second strand” refer to single-stranded polynucleotides. As described herein and commonly known in the art the complementary (matching) nucleotide of adenosine (A) is thymidine (T) and the complementary (matching) nucleotide of guanosine (G) is cytosine (C). Thus, a complement may include a sequence of nucleotides that base pair with corresponding complementary nucleotides of a second nucleic acid sequence. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often comprise nucleic acid sequences that are substantially complementary to each other.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g. chemical compounds including biomolecules or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, a protein or enzyme (e.g., a DNA polymerase).

As may be used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

Two or more associated species are “tethered”, “coated”, “attached”, or “immobilized” to one another or to a common solid or semisolid support (e.g. a receiving substrate). An association may refer to a relationship, or connection, between two entities. As used herein, an immobilized polynucleotide or an immobilized primer refers to a polynucleotide or a primer that is attached to a solid surface, such as a solid support. The immobilized polynucleotide and/or immobilized primer may be attached covalently (e.g. through a linker) or non-covalently to a solid support. In embodiments, immobilized polynucleotide and/or immobilized primer is covalently attached to a solid support.

As used herein, the terms “polynucleotide primer” and “primer” refers to any polynucleotide molecule that may hybridize to a polynucleotide template, be bound by a polymerase, and be extended in a template-directed process for nucleic acid synthesis. The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

As used herein, a platform primer is a primer oligonucleotide immobilized or otherwise bound to a solid support (i.e. an immobilized oligonucleotide). Examples of platform primers include P7 and P5 primers, or S1 and S2 sequences, or the reverse complements thereof. A “platform primer binding sequence” refers to a sequence or portion of an oligonucleotide that is capable of binding to a platform primer (e.g., the platform primer binding sequence is complementary to the platform primer). In embodiments, a platform primer binding sequence may form part of an adapter. In embodiments, a platform primer binding sequence is complementary to a platform primer sequence. In embodiments, a platform primer binding sequence is complementary to a primer.

The order of elements within a nucleic acid molecule is typically described herein from 5′ to 3′. In the case of a double-stranded molecule, the “top” strand is typically shown from 5′ to 3′, according to convention, and the order of elements is described herein with reference to the top strand.

The term “adapter” as used herein refers to any oligonucleotide that can be ligated to a nucleic acid molecule, thereby generating nucleic acid products that can be sequenced on a sequencing platform (e.g., an Illumina or Singular Genomics G4™ sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., P7 and P5 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing. In some embodiments, an adapter is hairpin adapter. In some embodiments, a hairpin adapter includes a single nucleic acid strand including a stem-loop structure. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, a method herein includes ligating a first adapter to a first end of a double stranded nucleic acid, and ligating a second adapter to a second end of a double stranded nucleic acid. In some embodiments, the first adapter and the second adapter are different. For example, in certain embodiments, the first adapter and the second adapter may include different nucleic acid sequences or different structures. In some embodiments, the first adapter is a Y-adapter and the second adapter is a hairpin adapter. In some embodiments, the first adapter is a hairpin adapter and a second adapter is a hairpin adapter. In certain embodiments, the first adapter and the second adapter may include different primer binding sites, different structures, and/or different capture sequences (e.g., a sequence complementary to a capture nucleic acid). In some embodiments, some, all or substantially all of the nucleic acid sequence of a first adapter and a second adapter are the same. In some embodiments, some, all or substantially all of the nucleic acid sequence of a first adapter and a second adapter are substantially different.

In some embodiments, a nucleic acid includes a capture nucleic acid. A capture nucleic acid refers to a nucleic acid that is attached to a substrate. In some embodiments, a capture nucleic acid includes a primer. In some embodiments, a capture nucleic acid is a nucleic acid configured to specifically hybridize to a portion of one or more nucleic acid templates (e.g., a template of a library). In some embodiments a capture nucleic acid configured to specifically hybridize to a portion of one or more nucleic acid templates is substantially complementary to a suitable portion of a nucleic acid template, or an amplicon thereof. In some embodiments a capture nucleic acid is configured to specifically hybridize to a portion of an adapter, or a portion thereof. In some embodiments a capture nucleic acid, or portion thereof, is substantially complementary to a portion of an adapter, or a complement thereof. In embodiments, a capture nucleic acid is a probe oligonucleotide. Typically, a probe oligonucleotide is complementary to a target polynucleotide or portion thereof, and further includes a label (such as a binding moiety) or is attached to a surface, such that hybridization to the probe oligonucleotide permits the selective isolation of probe-bound polynucleotides from unbound polynucleotides in a population. A probe oligonucleotide may or may not also be used as a primer.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate). A “canonical” nucleotide is an unmodified nucleotide.

In embodiments, the nucleotides of the present disclosure use a cleavable linker to attach the label to the nucleotide. The use of a cleavable linker ensures that the label can, if required, be removed after detection, avoiding any interfering signal with any labelled nucleotide incorporated subsequently. The use of the term “cleavable linker” is not meant to imply that the whole linker is required to be removed from the nucleotide base. The cleavage site can be located at a position on the linker that ensures that part of the linker remains attached to the nucleotide base after cleavage. The linker can be attached at any position on the nucleotide base provided that Watson-Crick base pairing can still be carried out. In the context of purine bases, it is preferred if the linker is attached via the 7-position of the purine or the preferred deazapurine analog, via an 8-modified purine, via an N-6 modified adenosine or an N-2 modified guanine. For pyrimidines, attachment is preferably via the 5-position on cytidine, thymidine or uracil and the N-4 position on cytosine.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

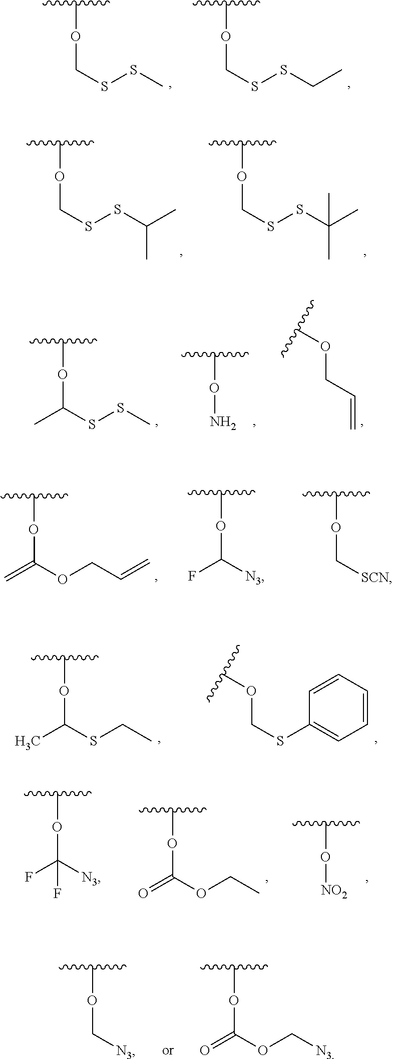

A label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogs include, without limitation, 7-deaza-adenine, 7-deaza-guanine, the analogs of deoxynucleotides shown herein, analogs in which a label is attached through a cleavable linker to the 5-position of cytosine or thymine or to the 7-position of deaza-adenine or deaza-guanine, and analogs in which a small chemical moiety is used to cap the OH group at the 3′-position of deoxyribose. Nucleotide analogs and DNA polymerase-based DNA sequencing are also described in U.S. Pat. No. 6,664,079, which is incorporated herein by reference in its entirety for all purposes.

The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analog such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analog. In general, the conditions under which a removable group is removed are compatible with a process employing the removable group (e.g., an amplification process or sequencing process).

As used herein, the terms “blocking moiety,” “reversible blocking group,” “reversible terminator” and “reversible terminator moiety” are used in accordance with their plain and ordinary meanings and refer to a cleavable moiety which does not interfere with incorporation of a nucleotide including it by a polymerase (e.g., DNA polymerase, modified DNA polymerase), but prevents further strand extension until removed (“unblocked”). For example, a reversible terminator may refer to a blocking moiety located, for example, at the 3′ position of the nucleotide and may be a chemically cleavable moiety such as an allyl group, an azidomethyl group or a methoxymethyl group, or may be an enzymatically cleavable group such as a phosphate ester. Suitable nucleotide blocking moieties are described in applications WO 2004/018497, U.S. Pat. Nos. 7,057,026, 7,541,444, WO 96/07669, U.S. Pat. Nos. 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group may be represented as —OR [reversible terminating (capping) group], wherein O is the oxygen atom of the 3′-OH of the pentose and R is the blocking group, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is

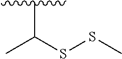 [SMILES: *C(C)SSC]

as described in U.S. Pat. No. 10,738,072, which is incorporated herein by reference for all purposes. In embodiments, the reversible terminator moiety is

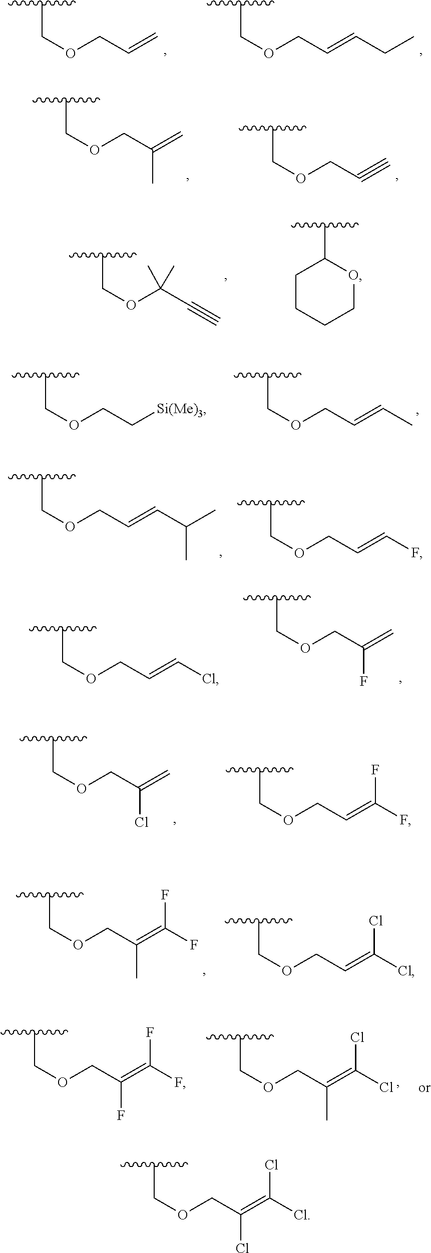

In embodiments, the reversible terminator moiety is

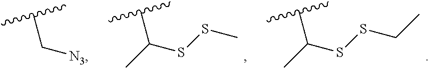

In some embodiments, a nucleic acid (e.g., an adapter or a primer) includes a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads including the barcode can be identified as originating from a single sample polynucleotide molecule on the basis of the barcode alone. In other embodiments, individual barcode sequences may be used more than once, but adapters including the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined.

In embodiments, a nucleic acid (e.g., an adapter or primer) includes a sample barcode. In general, a “sample barcode” is a nucleotide sequence that is sufficiently different from other sample barcode to allow the identification of the sample source based on sample barcode sequence(s) with which they are associated. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments, substantially degenerate sample barcodes may be known as random. In some embodiments, a sample barcode may include a nucleic acid sequence from within a pool of known sequences. In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides.

As used herein, the terms “denaturant” or plural “denaturants” are used in accordance with their plain and ordinary meanings and refer to an additive or condition that disrupts the base pairing between nucleotides within opposing strands of a double-stranded polynucleotide molecule. The term “denature” and its variants, when used in reference to any double-stranded polynucleotide molecule, or double-stranded polynucleotide sequence, includes any process whereby the base pairing between nucleotides within opposing strands of the double-stranded molecule, or double-stranded sequence, is disrupted. Typically, denaturation includes rendering at least some portion or region of two strands of the double-stranded polynucleotide molecule or sequence single-stranded or partially single-stranded. In some embodiments, denaturation includes separation of at least some portion or region of two strands of the double-stranded polynucleotide molecule or sequence from each other. Typically, the denatured region or portion is then capable of hybridizing to another polynucleotide molecule or sequence. Optionally, there can be “complete” or “total” denaturation of a double-stranded polynucleotide molecule or sequence. Complete denaturation conditions are, for example, conditions that would result in complete separation of a significant fraction (e.g., more than 10%, 20%, 30%, 40% or 50%) of a large plurality of strands from their extended and/or full-length complements. Typically, complete or total denaturation disrupts all of the base pairing between the nucleotides of the two strands with each other. Similarly, a nucleic acid sample is optionally considered fully denatured when more than 80% or 90% of individual molecules of the sample lack any double-strandedness (or lack any hybridization to a complementary strand).

Alternatively, the double-stranded polynucleotide molecule or sequence can be partially or incompletely denatured. A given nucleic acid molecule can be considered partially denatured when a portion of at least one strand of the nucleic acid remains hybridized to a complementary strand, while another portion is in an unhybridized state (even if it is in the presence of a complementary sequence). The unhybridized portion is optionally at least 5, 10, 15, 20, 50, or more nucleotides in length. The hybridized portion is optionally at least 5, 10, 15, 20, 50, or more nucleotides in length. Partial denaturation includes situations where some, but not all, of the nucleotides of one strand or sequence, are based paired with some nucleotides of the other strand or sequence within a double-stranded polynucleotide. In some embodiments, at least 20% but less than 100% of the nucleotide residues of one strand of the partially denatured polynucleotide (or sequence) are not base paired to nucleotide residues within the opposing strand. In embodiments, at least 50% of nucleotide residues within the double-stranded polynucleotide molecule (or double-stranded polynucleotide sequence) are in single-stranded (or unhybridized) from, but less than 20% or 10% of the residues are double-stranded.

Optionally, a nucleic acid sample can be considered to be partially denatured when a substantial fraction of individual nucleic acid molecules of the sample (e.g., above 20%, 30%, 50%, or 70%) are in a partially denatured state. Optionally less than a substantial amount of individual nucleic acid molecules in the sample are fully denatured, e.g., not more than 5%, 10%, 20%, 30% or 50% of the nucleic acid molecules in the sample. Under exemplary conditions at least 50% of the nucleic acid molecules of the sample are partly denatured, but less than 20% or 10% are fully denatured. In other situations, at least 30% of the nucleic acid molecules of the sample are partly denatured, but less than 10% or 5% are fully denatured. Similarly, a nucleic acid sample can be non-denatured when a minority of individual nucleic acid molecules in the sample are partially or completely denatured.

In an embodiment, partially denaturing conditions are achieved by maintaining the duplexes as a suitable temperature range. For example, the nucleic acid is maintained at temperature sufficiently elevated to achieve some heat-denaturation (e.g., above 45° C., 50° C., 55° C., 60° C., 65° C., or 70° C.) but not high enough to achieve complete heat-denaturation (e.g., below 95° C. or 90° C. or 85° C. or 80° C. or 75° C.). In an embodiment the nucleic acid is partially denatured using substantially isothermal conditions. Alternatively, chemical denaturation can be accomplished by contacting the double-stranded polynucleotide to be denatured with appropriate chemical denaturants, such as strong alkalis, strong acids, chaotropic agents, and the like and can include, for example, NaOH, urea, or guanidine-containing compounds. In some embodiments, partial or complete denaturation is achieved by exposure to chemical denaturants such as urea or formamide, with concentrations suitably adjusted, or using high or low pH (e.g., pH between 4-6 or 8-9). In embodiments, the denaturant is a buffered solution including betaine, dimethyl sulfoxide (DMSO), ethylene glycol, formamide, glycerol, guanidine thiocyanate, 4-methylmorpholine 4-oxide (NMO), or a mixture thereof. In embodiments, the first denaturant is a buffered solution including about 0% to about 50% dimethyl sulfoxide (DMSO); about 0% to about 50% ethylene glycol; about 0% to about 20% formamide; or about 0 to about 3M betaine, or a mixture thereof. In an embodiment herein, partial denaturation and/or amplification, including any one or more steps or methods described herein, can be achieved using a recombinase and/or single-stranded binding protein.

In some embodiments, complete or partial denaturation of a double-stranded polynucleotide sequence is accomplished by contacting the double-stranded polynucleotide sequence using appropriate denaturing agents. For example, the double-stranded polynucleotide can be subjected to heat-denaturation (also referred to interchangeably as thermal denaturation) by raising the temperature to a point where the desired level of denaturation is accomplished. In some embodiments, thermal denaturation of a double-stranded polynucleotide, includes adjusting the temperature to achieve complete separation of the two strands of the polynucleotide, such that 90% or greater of the strands are in single-stranded form across their entire length. A completely denatured double-stranded polynucleotide results in a separated first strand and a second strand, each of which is a single-stranded polynucleotide. In some embodiments, complete thermal denaturation of a polynucleotide molecule (or polynucleotide sequence) is accomplished by exposing the polynucleotide molecule (or sequence) to a temperature that is at least 5° C., 10° C., 15° C., 20° C., 25° C., 30° C., 50° C., or 100° C., above the calculated or predict melting temperature (Tm) of the polynucleotide molecule or sequence.

In some embodiments, complete or partial denaturation is accomplished by treating the double-stranded polynucleotide sequence to be denatured using a denaturant mixture including an SSB protein (e.g., T4 gp32 protein, T7 gene 2.5 SSB protein, or phi29 SSB protein, Thermococcus kodakarensis (KOD) SSB, Thermus thermophilus (TTH) SSB, Sulfolobus solfataricus (SSO) SSB, or Extreme Thermostable Single-Stranded DNA Binding Protein (ET-SSB)), a strand-displacing polymerase (e.g., Bst large fragment (Bst LF) polymerase, Bst 3.0 polymerase, Bst 2.0 polymerase, Bsu polymerase, SD polymerase, Vent exo-polymerase, Phi29 polymerase, or a mutant thereof), and one or more crowding agents (poly(ethylene glycol) (PEG), polyvinylpyrrolidone (PVP), bovine serum albumin (BSA), dextran, Ficoll (e.g., Ficoll 70 or Ficoll 400), glycerol, or a combination thereof). In embodiments, the crowding agent is poly(ethylene glycol) (e.g., PEG 200, PEG 600, PEG 800, PEG 2,050, PEG 4,600, PEG 6,000, PEG 8,000, PEG 10,000, PEG 20,000, or PEG 35,000), dextran sulfate, bovine pancreatic trypsin inhibitor (BPTI), ribonuclease A, lysozyme, β-lactoglobulin, hemoglobin, bovine serum albumin (BSA), or poly(sodium 4-styrene sulfonate) (PSS). In embodiments, the denaturant mixture including an SSB, a strand-displacing polymerase, and one or more crowding agents does not include a chemical denaturant (e.g., betaine, DMSO, ethylene glycol, formamide, guanidine thiocyanate, NMO, TMAC, or a mixture thereof).

In some embodiments, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing).

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. Coli DNA polymerase I, Bacteriophage T4 DNA polymerase, Sequenase, Taq DNA polymerase, DNA polymerase from Bacillus stearothermophilus, Bst 2.0 DNA polymerase, 9ºN polymerase (exo-) A485L/Y409V, Phi29 DNA Polymerase (429 DNA Polymerase), T7 DNA polymerase, DNA polymerase II, DNA polymerase III holoenzyme, DNA polymerase IV, DNA polymerase V, Vent® DNA polymerase, Therminator™ II DNA Polymerase, Therminator™ III DNA Polymerase, or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol a DNA polymerase, Pol δ DNA polymerase, Pol ε DNA polymerase, Pol η DNA polymerase, Pol t DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g. Therminator γ, 9ºN polymerase (exo-), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044).

As used herein, the term “exonuclease activity” is used in accordance with its ordinary meaning in the art, and refers to the removal of a nucleotide from a nucleic acid by an enzyme (e.g. DNA polymerase, a lambda exonuclease, Exo I, Exo III, T5, Exo V, Exo VII or the like). For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′->5′ direction, releasing deoxyribonucleoside 5′-monophosphates one after another. Methods for quantifying exonuclease activity are known in the art, see for example Southworth et al, PNAS Vol 93, 8281-8285 (1996). In embodiments, 5′-3′ exonuclease activity refers to the successive removal of nucleotides in double-stranded DNA in a 5′->3′ direction. In embodiments, the 5′-3′ exonuclease is lambda exonuclease. For example, lambda exonuclease catalyzes the removal of 5′ mononucleotides from duplex DNA, with a preference for 5′ phosphorylated double-stranded DNA. In other embodiments, the 5′-3′ exonuclease is E. coli DNA Polymerase I.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. For example, a chemical reagent may selectively modify one nucleotide type in that it reacts with one nucleotide type (e.g., cytosines) and not other nucleotide types (e.g., adenine, thymine, or guanine). When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the term “template polynucleotide” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In the context of selective sequencing, “target polynucleotide(s)” refers to the subset of polynucleotide(s) to be sequenced from within a starting population of polynucleotides.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g. apoptosis or necrosis) or cell shedding, releasing polynucleotides into surrounding body fluids or into circulation. Accordingly, cell-free polynucleotides may be isolated from a non-cellular fraction of blood (e.g. serum or plasma), from other bodily fluids (e.g. urine), or from non-cellular fractions of other types of samples

As used herein, the terms “specific”, “specifically”, “specificity”, or the like of a compound refers to the compound's ability to cause a particular action, such as binding, to a particular molecular target with minimal or no action to other proteins in the cell.

As used herein, the terms “bind” and “bound” are used in accordance with their plain and ordinary meanings and refer to an association between atoms or molecules. The association can be direct or indirect. For example, bound atoms or molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

“Specific binding” is where the binding is selective between two molecules. A particular example of specific binding is that which occurs between an antibody and an antigen. Typically, specific binding can be distinguished from non-specific when the dissociation constant (KD) is less than about 1×10⁻⁵ M or less than about 1×10⁶ M or 1×10⁷ M. Specific binding can be detected, for example, by ELISA, immunoprecipitation, coprecipitation, with or without chemical crosslinking, two-hybrid assays and the like. In embodiments, the K_{D} (equilibrium dissociation constant) between two specific binding molecules is less than 10⁻⁶ M, less than 10⁻⁷M, less than 10⁻⁸ M, less than 10⁻⁹M, less than 10⁻⁹ M, less than 10⁻¹¹ M, or less than about 10⁻¹² M or less.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. In embodiments, the solid substrate is in the form of a chip, a bead, a well, a capillary tube, a slide, a wafer, a filter, a fiber, a porous media, or a column. In embodiments, the solid substrate is gold, quartz, silica, plastic, glass, diamond, silver, metal, or polypropylene. In embodiments, the solid substrate is porous.

As used herein, the terms “solid support” and “substrate” and “solid surface” refers to discrete solid or semi-solid surfaces to which a plurality of primers may be attached. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may include a discrete particle that may be spherical (e.g., microspheres) or have a non-spherical or irregular shape, such as cubic, cuboid, pyramidal, cylindrical, conical, oblong, or disc-shaped, and the like. Solid supports may be in the form of discrete particles, which alone does not imply or require any particular shape. The term “particle” means a small body made of a rigid or semi-rigid material. The body can have a shape characterized, for example, as a sphere, oval, microsphere, or other recognized particle shape whether having regular or irregular dimensions. As used herein, the term “discrete particles” refers to physically distinct particles having discernible boundaries. The term “particle” does not indicate any particular shape. The shapes and sizes of a collection of particles may be different or about the same (e.g., within a desired range of dimensions, or having a desired average or minimum dimension). A particle may be substantially spherical (e.g., microspheres) or have a non-spherical or irregular shape, such as cubic, cuboid, pyramidal, cylindrical, conical, oblong, or disc-shaped, and the like. In embodiments, the particle has the shape of a sphere, cylinder, spherocylinder, or ellipsoid. Discrete particles collected in a container and contacting one another will define a bulk volume containing the particles, and will typically leave some internal fraction of that bulk volume unoccupied by the particles, even when packed closely together. In embodiments, cores and/or core-shell particles are approximately spherical. As used herein the term “spherical” refers to structures which appear substantially or generally of spherical shape to the human eye, and does not require a sphere to a mathematical standard. In other words, “spherical” cores or particles are generally spheroidal in the sense of resembling or approximating to a sphere. In embodiments, the diameter of a spherical core or particle is substantially uniform, e.g., about the same at any point, but may contain imperfections, such as deviations of up to 1, 2, 3, 4, 5 or up to 10%. Because cores or particles may deviate from a perfect sphere, the term “diameter” refers to the longest dimension of a given core or particle. Likewise, polymer shells are not necessarily of perfect uniform thickness all around a given core. Thus, the term “thickness” in relation to a polymer structure (e.g., a shell polymer of a core-shell particle) refers to the average thickness of the polymer layer.

A solid support may further include a polymer or hydrogel on the surface to which the primers are attached (e.g., the primers are covalently attached to the polymer, wherein the polymer is in direct contact with the solid support). Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. The solid supports for some embodiments have at least one surface located within a flow cell. The solid support, or regions thereof, can be substantially flat. The solid support can have surface features such as wells, pits, channels, ridges, raised regions, pegs, posts or the like. The term solid support is encompassing of a substrate (e.g., a flow cell) having a surface including a polymer coating covalently attached thereto. In embodiments, the solid support is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g. steel, gold, silver, aluminum, silicon and copper). In some embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In some embodiments a substrate includes a magnetic material (e.g., iron, nickel, cobalt, platinum, aluminum, and the like). In certain embodiments a substrate includes a magnetic bead (e.g., DYNABEADS®, hematite, AMPure XP). Magnets can be used to purify and/or capture nucleic acids bound to certain substrates (e.g., substrates including a metal or magnetic material).

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

Polymers can be hydrophilic, hydrophobic, or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers. Hydrogels can contain over 99% water and may include natural or synthetic polymers, or a combination thereof. Hydrogels also possess a degree of flexibility very similar to natural tissue, due to their significant water content. A detailed description of suitable hydrogels may be found in published U.S. patent application 2010/0055733, herein incorporated by reference. By “hydrogel subunits” or “hydrogel precursors” is meant hydrophilic monomers, prepolymers, or polymers that can be crosslinked, or “polymerized”, to form a three-dimensional (3D) hydrogel network. In some embodiments, the alternating layers of polymeric gels described herein are hydrogels. Hydrogels may be prepared by cross-linking hydrophilic biopolymers or synthetic polymers. Thus, in some embodiments, the hydrogel may include a crosslinker. As used herein, the term “crosslinker” refers to a molecule that can form a three-dimensional network when reacted with the appropriate base monomers. Examples of the hydrogel polymers, which may include one or more crosslinkers, include but are not limited to, hyaluronans, chitosans, agar, heparin, sulfate, cellulose, alginates (including alginate sulfate), collagen, dextrans (including dextran sulfate), pectin, carrageenan, polylysine, gelatins (including gelatin type A), agarose, (meth)acrylate-oligolactide-PEO-oligolactide-(meth)acrylate, PEO—PPO-PEO copolymers (Pluronics), poly(phosphazene), poly(methacrylates), poly(N-vinylpyrrolidone), PL(G)A-PEO-PL(G)A copolymers, poly(ethylene imine), polyethylene glycol (PEG)-thiol, PEG-acrylate, acrylamide, N,N′-bis(acryloyl)cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof. Thus, for example, a combination may include a polymer and a crosslinker, for example polyethylene glycol (PEG)-thiol/PEG-acrylate, acrylamide/N,N′-bis(acryloyl)cystamine (BACy), or PEG/polypropylene oxide (PPO). In embodiments, the hydrogel includes chemical crosslinks (e.g., intermolecular or intramolecular joining of two or more molecules by a covalent bond) and may be referred to as a chemical hydrogel. In embodiments, the hydrogel includes physical crosslinks (e.g., intermolecular or intramolecular joining of two or more molecules by a non-covalent bond) and may be referred to as a physical hydrogel. In embodiments, the physical hydrogel include one or more crosslinks including hydrogen bonds, hydrophobic interactions, and/or polymer chain entanglements.

The term “array” as used herein, refers to a container (e.g., a microplate, tube, or flow cell) including a plurality of features (e.g., wells). For example, an array may include a container with a plurality of wells. In embodiments, the array is a microplate. In embodiments, the array is a flow cell. In embodiments, the solid support includes about 0.2 wells to about 4.0 wells per μm². In embodiments, the solid support includes about 0.2 wells to about 0.8 wells per μm². In embodiments, the solid support includes about 0.8 wells to about 1.2 wells per μm². In embodiments, the solid support includes about 1.2 wells to about 2.0 wells per μm². In embodiments, the solid support includes about 2.0 wells to about 3.0 wells per μm². In embodiments, the solid support includes about 3.0 wells to about 4.0 wells per μm². In embodiments, the solid support includes about 0.2, about 0.4, about 0.6, about 0.8, about 1.0, about 1.2, about 1.4, about 1.6, about 1.8, about 2.0, about 2.2, about 2.4, about 2.6, about 2.8, about 3.0, about 3.2, about 3.4, about 3.6 about 3.8, or about 4.0 wells per μm². In embodiments, the solid support includes about 0.2 wells per μm². In embodiments, the solid support includes about 0.6 wells per μm². In embodiments, the solid support includes about 1.0 wells per μm². In embodiments, the solid support includes about 1.2 wells per μm². In embodiments, the solid support includes about 1.8 wells per μm². In embodiments, the solid support includes about 2.4 wells per μm². In embodiments, the solid support includes about 3.0 wells per μm². In embodiments, the solid support includes about 4.0 wells per μm².

As used herein, the term “sequencing reaction mixture” is used in accordance with its plain and ordinary meaning and refers to an aqueous mixture that contains the reagents necessary to allow dNTP or dNTP analog to add a nucleotide to a DNA strand by a DNA polymerase. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-pipcrazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

As used herein, the term “sequencing cycle” is used in accordance with its plain and ordinary meaning and refers to incorporating one or more nucleotides (e.g., nucleotide analogs) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. An “extension strand” is formed as the one or more nucleotides are incorporated into a complementary polynucleotide hybridized to a template nucleic acid. The extension strand is complementary to the template nucleic acid. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes and other substances can be removed between steps by washing. Cycles may include repeating these steps, and the sequence of each cluster is read over the multiple repetitions.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. The sequence reads are optionally stored in an appropriate data structure for further evaluation. In embodiments, a first sequencing reaction can generate a first sequencing read. The first sequencing read can provide the sequence of a first region of the polynucleotide fragment. In embodiments, a second sequencing primer can initiate sequencing at a second location on the nucleic acid template. The second location can be distinct from the first location. In some cases, a 3′ terminal nucleotide of the second primer can hybridize to a location that is more than 5 nucleotides away from a binding site of a 3′ terminal nucleotide of the first primer. The second sequencing reaction can generate a second sequencing read. The second sequencing read can provide the sequence of a second region of the nucleic acid template which is distinct from the first region of the nucleic acid template. In some embodiments, the nucleic acid template is optionally subjected to one or more additional rounds of sequencing using additional sequencing primers, thereby generating additional sequencing reads.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

Complementary single stranded nucleic acids and/or substantially complementary single stranded nucleic acids can hybridize to each other under hybridization conditions, thereby forming a nucleic acid that is partially or fully double stranded. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

“Hybridize” shall mean the annealing of a nucleic acid sequence to another nucleic acid sequence (e.g., one single-stranded nucleic acid (such as a primer) to another nucleic acid) based on the well-understood principle of sequence complementarity. In an embodiment the other nucleic acid is a single-stranded nucleic acid. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analog capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acid. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double stranded portion of nucleic acid.

A nucleic acid can be amplified by a suitable method. The term “amplified” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction includes a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that includes a polymerase chain reaction (PCR). Conditions conducive to amplification (i.e., amplification conditions) are well known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

As used herein, bridge-PCR (bPCR) amplification is a method for solid-phase amplification as exemplified by the disclosures of U.S. Pat. Nos. 5,641,658; 7,115,400; and U.S. Patent Publ. No. 2008/0009420, each of which is incorporated herein by reference in its entirety. Bridge-PCR involves repeated polymerase chain reaction cycles, cycling between denaturation, annealing, and extension conditions and enables controlled, spatially-localized, amplification, to generate amplification products (e.g., amplicons) immobilized on a solid support in order to form arrays comprised of colonies (or “clusters”) of immobilized nucleic acid molecule.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

A nucleic acid can be amplified by a thermocycling method. In some embodiments, amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution based primers can be used. Non-limiting examples of solid phase nucleic acid amplification reactions include interfacial amplification, bridge PCR amplification, emulsion PCR, WildFire amplification (e.g., US patent publication US20130012399), the like or combinations thereof.

In certain embodiments, a nucleic acid template including a complementary forward and reverse stand of a double stranded nucleic acid, a hairpin adapter on one end, and a Y adapter on the other end, is amplified by bridge PCR amplification. The bridge PCR amplification process of a nucleic acid template including such a configuration is mechanistically distinct from a bridge amplification that takes place for a single stranded nucleic acid template containing no internal complementary regions. For example, after a denaturation step in bridge PCR of a nucleic acid template including such a configuration, amplicons can preferentially form an intramolecular double-stranded region as opposed to staying double-stranded at an intermolecular scale. This enables a free 3′ end at the Y-adapter end, which is available for re-priming with additional solid-phase primers.

In some embodiments, a nucleic acid, adapter, oligonucleotide probe, template and/or substrate includes a binding motif. In some embodiments a binding motif is one member of a binding pair where each member of the binding pair can bind to each other specifically and with relatively high affinity. For example, typical binding pairs bind to each other with a Kd of less than about 10 μM, 5 μM, 1 μM, 500 nM, 250 nM, 100 nM, 75 nM, 50 nM, 25 nM, 15 nM, 10 nM, 5 nM, 1 nM, or less than about 0.1 nM. In some embodiments a binding pair includes at least two members (e.g., molecules) that bind non-covalently to (e.g., associate with) each other. Members of a binding pair often bind specifically to each other. In certain embodiments, members of a binding pair bind reversibly to each other, for example where the association of two members of a binding pair can be dissociated by a suitable method. Non-limiting examples of a binding pair include antibody/antigen, antibody/antibody, antibody/antibody fragment, antibody/antibody receptor, antibody/protein A, antibody/protein G, hapten/anti-hapten, biotin/avidin, biotin/streptavidin, folic acid/folate binding protein, receptor/ligand, vitamin B12/intrinsic factor, analogs thereof, derivatives thereof, binding portions thereof, the like or combinations thereof. Non-limiting examples of a binding motif or a member of a binding pair include an antibody, antibody fragment, reduced antibody, chemically modified antibody, antibody receptor, Fab, Fab′, F(ab′)2, Fv fragment, single-chain Fv (scFv), diabody (Dab), synbody, TandAbs, nanobodies, BiTEs, SMIPs, DARPins, DNLs, affibodies, Duocalins, adnectins, fynomers, Kunitz Domains AlbudAbs, DARTs, DVD-IG, Covx-bodies, peptibodies, scFv-Igs, SVD-Igs, dAb-Igs, Knob-in-Holes, triomAbs, an antigen, hapten, anti-hapten, aptamer, receptor, ligand, metal ion, avidin, streptavidin, neutravidin, biotin, B12, intrinsic factor, analogs thereof, derivatives thereof, binding portions thereof, the like or combinations thereof.

In some embodiments a nucleic acid is directly or indirectly bound (e.g., covalently or non-covalently bound) to a suitable substrate. In certain embodiments a substrate includes a surface (e.g., a surface of a flow cell, a surface of a tube, a surface of a chip), for example a metal surface (e.g. steel, gold, silver, aluminum, silicon and copper). In some embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In some embodiments a substrate includes a magnetic material (e.g., iron, nickel, cobalt, platinum, aluminum, and the like). In certain embodiments a substrate includes a magnetic bead (e.g., DYNABEADS®, hematite, AMPure XP. Magnets can be used to purify and/or capture nucleic acids bound to certain substrates (e.g., substrates including a metal or magnetic material).

Provided herein are methods and compositions for analyzing a sample (e.g., sequencing nucleic acids within a sample). A sample (e.g., a sample including nucleic acid) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, car, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-frec). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, car, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid).

In some embodiments, a sample includes nucleic acid, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

The term “nucleobase” or “base” as used herein refers to a purine or pyrimidine compound, or a derivative thereof, that may be a constituent of nucleic acid (i.e., DNA or RNA, or a derivative thereof). In embodiments, the nucleobase is a divalent purine or pyrimidine, or derivative thereof. In embodiments, the nucleobase is a monovalent purine or pyrimidine, or derivative thereof. In embodiments, the base is a derivative of a naturally occurring DNA or RNA base (e.g., a base analog). In embodiments the base is a hybridizing base. In embodiments the base hybridizes to a complementary base. In embodiments, the base is capable of forming at least one hydrogen bond with a complementary base (e.g., adenine hydrogen bonds with thymine, adenine hydrogen bonds with uracil, guanine pairs with cytosine). Non-limiting examples of a base includes cytosine or a derivative thereof (e.g., cytosine analog), guanine or a derivative thereof (e.g., guanine analog), adenine or a derivative thereof (e.g., adenine analog), thymine or a derivative thereof (e.g., thymine analog), uracil or a derivative thereof (e.g., uracil analog), hypoxanthine or a derivative thereof (e.g., hypoxanthine analog), xanthine or a derivative thereof (e.g., xanthine analog), 7-methylguanine or a derivative thereof (e.g., 7-methylguanine analog), deaza-adenine or a derivative thereof (e.g., deaza-adenine analog), deaza-guanine or a derivative thereof (e.g., deaza-guanine), deaza-hypoxanthine or a derivative thereof, 5,6-dihydrouracil or a derivative thereof (e.g., 5,6-dihydrouracil analog), 5-methylcytosine or a derivative thereof (e.g., 5-methylcytosine analog), or 5-hydroxymethylcytosine or a derivative thereof (e.g., 5-hydroxymethylcytosine analog) moieties. In embodiments, the base is adenine, guanine, uracil, cytosine, thymine, hypoxanthine, xanthine, theobromine, caffeine, uric acid, or isoguanine, which may be optionally substituted or modified. In embodiments, the base is adenine, guanine, hypoxanthine, xanthine, theobromine, caffeine, uric acid, or isoguanine, which may be optionally substituted or modified.

As used herein, a “methylated nucleotide” or a “methylated nucleobase” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA. Typical nucleoside bases for DNA are thymine, adenine, cytosine and guanine. Typical bases for RNA are uracil, adenine, cytosine and guanine.

As used herein, a “methyltransferase reagent” refers to a reagent that can transfer or catalyze transfer of a methyl moiety to a compound such as a nucleotide or nucleic acid molecule. Typically a methyltransferase reagent can transfer the methyl moiety with base specificity. Exemplary methyltransferase reagents are DNA methyltransferases, as known in the art. In some embodiments, the DNA methyltransferase is DNMT1. In embodiments, the methyltransferase reagent is DNMT1, M.SssI, DNMT, or a homolog or mutant thereof. In embodiments, the methyltransferase reagent is DNMT3a, DNMT3b, DRM2, MET1, and CMT3, or a homolog or mutant thereof.

As used herein, the term “CpG island” refers to a genomic DNA region that contains a high percentage of CpG sites relative to the average genomic CpG incidence (per same species, per same individual, or per subpopulation (e.g., strain, ethnic subpopulation, or the like). Various parameters and definitions for CpG islands exist; for example, in some embodiments, CpG islands are defined as having a GC percentage that is greater than 50% and with an observed/expected CpG ratio that is greater than 60% (Gardiner-Garden et al. (1987) J Mol. Biol. 196:261-282; Baylin et al. (2006) Nat. Rev. Cancer 6:107-116; Irizarry et al. (2009) Nat. Genetics 41:178-186; each herein incorporated by reference in its entirety). In some embodiments, CpG islands may have a GC content >55% and observed CpG/expected CpG of 0.65 (Takai et al. (2007) PNAS 99:3740-3745; herein incorporated by reference in its entirety). Various parameters also exist regarding the length of CpG islands. As used herein, CpG islands may be less than 100 bp; 100-200 bp, 200-300 bp, 300-500 bp, 500-750 bp; 750-1000 bp; 1000 or more bp in length. In some embodiments, CpG islands show altered methylation patterns (e.g., altered 5hmC patterns) relative to controls (e.g., altered 5hmC methylation in cancer subjects relative to subjects without cancer; tissue-specific altered 5hmC patterns; altered 5hmC patterns in biological samples from subjects with a neoplasia or tumor relative to subjects without a neoplasia or tumor. In some embodiments, altered methylation involves increased incidence of 5hmC. In some embodiments, altered methylation involves decreased incidence of 5hmC.

As used herein, the term “CpG shore” or “CpG island shore” refers to a genomic region external to a CpG island that is or that has potential to have altered methylation (e.g., 5hmC) patterns (see, e.g., Irizarry et al. (2009) Nat. Genetics 41:178-186; herein incorporated by reference in its entirety). CpG island shores may show altered methylation (e.g., 5hmC) patterns relative to controls (e.g., altered 5hmC in cancer subjects relative to subjects without cancer; tissue-specific altered 5hmC patterns; altered 5hmC in biological samples from subjects with neoplasia or tumor relative to subjects without neoplasia or tumor. In some embodiments, altered methylation involves increased incidence of 5hmC. In some embodiments, altered methylation involves decreased incidence of 5hmC. CpG island shores may be located in various regions relative to CpG islands (see, e.g., Irizarry et al. (2009) Nat. Genetics 41; 178-186; herein incorporated by reference in its entirety). Accordingly, in some embodiments, CpG island shores are located less than 100 bp; 100-250 bp; 250-500 bp; 500-1000 bp; 1000-1500 bp; 1500-2000 bp; 2000-3000 bp; 3000 bp or more away from a CpG island.

As used herein, the “methylation state” or “methylation pattern” of a target nucleic acid molecule refers to the presence of absence of one or more methylated nucleotide bases in a target nucleic acid molecule. For example, a target nucleic acid molecule containing a methylated cytosine is considered methylated (i.e., the methylation state of the target nucleic acid molecule is methylated). A target nucleic acid molecule that does not contain any methylated nucleotides is considered unmethylated. Similarly, the methylation state of a nucleotide locus in a target nucleic acid molecule refers to the presence or absence of a methylated nucleotide at a particular locus in the target nucleic acid molecule. For example, the methylation state of a cytosine at the 7th nucleotide in a target nucleic acid molecule is methylated when the nucleotide present at the 7th nucleotide in the target nucleic acid molecule is 5-methylcytosine. Similarly, the methylation state of a cytosine at the 7th nucleotide in a target nucleic acid molecule is unmethylated when the nucleotide present at the 7th nucleotide in the target nucleic acid molecule is cytosine (and not 5-methylcytosine).

The term “cytosine nucleobase” as used herein refers to nucleotides, nucleosides, nucleotide triphosphates and the like which include cytosine (i.e., 4-amino-3H-pyrimidin-2-one) as the base. A cytosine nucleobase may be an unmodified cytosine nucleobase (e.g., a cytosine (C)), or a cytosine nucleobase may be a modified cytosine nucleobase (e.g., a 5-methylcytosine (5mC), 5-hydroxymethyl cytosine (5hmC), 5-formylcytosine (5fC), 5-carboxylcytosine (5caC), or β-glucosyl-5-hydroxymethylcytosine (5gmC)). Thus, for example, where embodiments describe converting an unmodified cytosine nucleobase to a uracil nucleobase, or uracil nucleobase analog, it will be understood that the term unmodified cytosine nucleobase does not include cytosine residues that are methylated at the 5-position of the cytosine base (e.g., a bisulfite-resistant modified cytosine nucleobase such as 5-methylcytosine), unless specifically indicated to the contrary. In some embodiments, the term cytosine nucleobase can refer to a base structure that is common between unmodified cytosine nucleobases and modified cytosine nucleobases, including bisulfite-resistant cytosine analogs, as described in detail herein.

The term “conversion” or “converted” as used herein in reference to a chemically modified nucleobase (e.g., 5-methylcytosine and 5-hydroxymethylcytosine) refers to the transformation of nucleobase to a different nucleobase. A “conversion agent” as used herein refers to a chemical or enzymatic agent that catalyzes the conversion of a nucleobase to a different nucleobase. For example, a conversion agent may catalyze the deamination of an unmodified cytosine nucleobase to a uracil nucleobase. In embodiments, a converted nucleobase is distinguishable from the modified nucleobase. For example, provided herein are methods for converting a modified cytosine (e.g., 5-methylcytosine, 5-hydroxymethylcytosine, 5-carboxylcytosine) to a uracil nucleobase analog (e.g., DHU). In embodiments, the unmodified cytosine nucleobase is converted to a uracil nucleobase (e.g., via bisulfite conversion, wherein the conversion agent is sodium bisulfite).

The term “cytosine mismatch” as used herein refers to a first nucleic acid sequence hybridized to a second nucleic acid sequence, wherein the cytosine nucleobase(s) does not form a Watson-Crick base pair with a guanine nucleobase(s). For example, a first strand having a cytosine nucleobase will form a cytosine mismatch with a second strand having a uracil nucleobase, or uracil nucleobase analog, at the complementary position. In embodiments, the uracil nucleobase analog is dihydrouridine (DHU).

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics™ such as the G4™ sequencing platform, Illumina™, Inc. (e.g., HiSeq™, MiSeq™, NextSeq™, or NovaSeq™ systems), Life Technologies™ (e.g., ABI PRISM™, or SOLID™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

As used herein, a “plurality” refers to two or more.

As used herein the terms “automated” and “semi-automated” mean that the operations are performed by system programming or configuration with little or no human interaction once the operations are initiated, or once processes including the operations are initiated.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Compositions & Kits

In an aspect is provided a polynucleotide including a first nucleic acid sequence including one or more methylated cytosine nucleobases hybridized to a second nucleic acid sequence including one or more uracil nucleobases, wherein the polynucleotide includes one or more cytosine mismatches. In embodiments, the first nucleic acid sequence and the second nucleic acid sequence may be linked together (e.g., with a hairpin adapter).

In an aspect is provided a polynucleotide including, from 5′ to 3′, a first primer binding sequence, a first template single-stranded nucleic acid sequence including one or more modified cytosine nucleobases, an adapter, a second template single-stranded nucleic acid, and a second primer binding sequence, wherein the first template single-stranded nucleic acid sequence is hybridized to a blocking nucleic acid sequence; and the second template single-stranded nucleic acid includes one or more modified cytosine nucleobases.

In an aspect is provided a polynucleotide including, from 5′ to 3′, a first primer binding sequence, a first template single-stranded nucleic acid sequence, an adapter, a second template single-stranded nucleic acid, and a second primer binding sequence, wherein the second template single-stranded nucleic acid sequence is hybridized to a blocking nucleic acid sequence; and the first template single-stranded nucleic acid includes one or more modified cytosine nucleobases.

In an aspect is provided a polynucleotide including, from 5′ to 3′, a first primer binding sequence, a first template single-stranded nucleic acid sequence including an unmodified cytosine nucleobase, an adapter, a second template single-stranded nucleic acid, and a second primer binding sequence, wherein the first template single-stranded nucleic acid sequence is hybridized to a blocking nucleic acid sequence; and the second template single-stranded nucleic acid comprises a modified cytosine nucleobase.

In an aspect is provided a polynucleotide including, from 5′ to 3′, a first primer binding sequence, a first template single-stranded nucleic acid sequence, an adapter, a second template single-stranded nucleic acid, and a second primer binding sequence, wherein the second template single-stranded nucleic acid sequence is hybridized to a blocking nucleic acid sequence; and the first template single-stranded nucleic acid includes a modified cytosine nucleobase.

In embodiments, the adapter is a hairpin adapter (e.g., a hairpin adapter as described herein). In embodiments, the adapter is a Y-adapter (e.g., a Y-adapter as described herein).

In certain embodiments, presented herein are compositions for conducting a method described herein, and including one or more elements thereof. In some embodiments, a composition includes (i) a template nucleic acid including sequences of a first strand of a Y-adapter, a forward strand (e.g., a first strand) of the double stranded nucleic acid including one or more modified cytosine nucleobases, a hairpin adapter, a reverse strand (e.g., second strand) of the double stranded nucleic acid and a second strand of the Y-adapter arranged in a 5′ to 3′ direction; wherein the template is attached to a substrate. In embodiments, the composition includes (ii) a primer hybridized to a loop of the hairpin adapter; wherein the template is attached to a substrate. In some embodiments, the substrate is a surface of a flow cell. In some embodiments, the substrate is a polymer coated surface of a flow cell. In embodiments, the substrate is a polymer coated particle (e.g., a polymer coated nanoparticle). In embodiments, the composition includes the complement of the template nucleic acid including sequences of a first strand of a Y-adapter, a forward strand (e.g., a first strand) of the double stranded nucleic acid, a hairpin adapter, a reverse strand (e.g., second strand) of the double stranded nucleic acid and a second strand of the Y-adapter arranged in a 5′ to 3′ direction wherein the complement of the template is attached to a substrate. In embodiments, the substrate includes a glass surface including a polymer coating. In embodiments, the substrate is glass or quartz, such as a microscope slide, having a surface that is uniformly silanized. This may be accomplished using conventional protocols, such as those described in Beattie et a (1995), Molecular Biotechnology, 4: 213. Such a surface is readily treated to permit end-attachment of oligonucleotides (e.g., forward and reverse primers) prior to amplification. In embodiments the substrate surface further includes a polymer coating, which contains functional groups capable of immobilizing primers. In some embodiments, the substrate includes a patterned surface suitable for immobilization of primers in an ordered pattern. A patterned surface refers to an arrangement of different regions in or on an exposed layer of a substrate. For example, one or more of the regions can be features where one or more primers are present. The features can be separated by interstitial regions where capture primers are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. In some embodiments, the primers are randomly distributed upon the substrate. In some embodiments, the primers are distributed on a patterned surface.

In embodiments, the template nucleic acid is immobilized on the substrate via a first linker and the complement of the template nucleic acid is immobilized to the substrate via a second linker. The linkers may also include spacer nucleotides. Including spacer nucleotides in the linker puts the template nucleic acid in an environment having a greater resemblance to free solution. This can be beneficial, for example, in enzyme-mediated reactions such as sequencing-by-synthesis. It is believed that such reactions suffer less steric hindrance issues that can occur when the template nucleic acid is directly attached to the solid support or is attached through a very short linker (e.g., a linker comprising about 1 to 3 carbon atoms). Spacer nucleotides form part of the immobilized template nucleic acid but do not participate in any reaction carried out on or with the polynucleotide (e.g. a hybridization or amplification reaction). In embodiments, the spacer nucleotides include 1 to 20 nucleotides. In embodiments, the linker includes 10 spacer nucleotides. In embodiments, the linker includes 12 spacer nucleotides. In embodiments, the linker includes 15 spacer nucleotides. It is preferred to use polyT spacers, although other nucleotides and combinations thereof can be used. In embodiments, the linker includes 10, 11, 12, 13, 14, or 15 T spacer nucleotides. In embodiments, the linker includes 12 T spacer nucleotides. Spacer nucleotides are typically included at the 5′ ends of polynucleotides which are attached to a suitable support. Attachment can be achieved via a phosphorothioate present at the 5′ end of the polynucleotide, an azide moiety, a dibenzocyclooctyne (DBCO) moiety, or any other bioconjugate reactive moiety. The linker may be a carbon-containing chain such as those of formula —(CH₂)n- wherein “n” is from 1 to about 1000. However, a variety of other linkers may be used so long as the linkers are stable under conditions used in DNA sequencing. In embodiments, the linker includes polyethylene glycol (PEG) having a general formula of —(CH₂—CH₂—O)m-, wherein m is from about 1 to 500, 1 to 100, or 1 to 12.

In embodiments, the linker, or the immobilized oligonucleotides (e.g., primers) include a cleavable site. In embodiments, the blocking primer includes a cleavable site. In embodiments, the adapter includes a cleavable site. In embodiments, a cleavable site is a location which allows controlled cleavage of the immobilized polynucleotide strand (e.g., the linker, the primer, or the polynucleotide) by chemical, enzymatic or photochemical means. In embodiments, the cleavable site includes one or more deoxyuracil nucleobases (dUs).

Any suitable enzymatic, chemical, or photochemical cleavage reaction may be used to cleave the cleavable site. The cleavage reaction may result in removal of a part or the whole of the strand being cleaved. Suitable cleavage means include, for example, restriction enzyme digestion, in which case the cleavable site is an appropriate restriction site for the enzyme which directs cleavage of one or both strands of a duplex template; RNase digestion or chemical cleavage of a bond between a deoxyribonucleotide and a ribonucleotide, in which case the cleavable site may include one or more ribonucleotides; chemical reduction of a disulfide linkage with a reducing agent (e.g., THPP or TCEP), in which case the cleavable site should include an appropriate disulfide linkage; chemical cleavage of a diol linkage with periodate, in which case the cleavable site should include a diol linkage; generation of an abasic site and subsequent hydrolysis, etc. In embodiments, the cleavable site is included in the surface immobilized primer (e.g., within the polynucleotide sequence of the primer). In embodiments, the linker, the primer, or the first or second polynucleotide includes a diol linkage which permits cleavage by treatment with periodate (e.g., sodium periodate). It will be appreciated that more than one diol can be included at the cleavable site. One or more diol units may be incorporated into a polynucleotide using standard methods for automated chemical DNA synthesis. Polynucleotide primers including one or more diol linkers can be conveniently prepared by chemical synthesis. The diol linker is cleaved by treatment with any substance which promotes cleavage of the diol (e.g., a diol-cleaving agent). In embodiments, the diol-cleaving agent is periodate, e.g., aqueous sodium periodate (NaIO₄). Following treatment with the diol-cleaving agent (e.g., periodate) to cleave the diol, the cleaved product may be treated with a “capping agent” in order to neutralize reactive species generated in the cleavage reaction. Suitable capping agents for this purpose include amines, e.g., ethanolamine or propanolamine. In embodiments, cleavage may be accomplished by using a modified nucleotide as the cleavable site (e.g., uracil, 8oxoG, 5-mC, 5-hmC) that is removed or nicked via a corresponding DNA glycosylase, endonuclease, or combination thereof.

In embodiments, each of the plurality of immobilized oligonucleotides (e.g., immobilized primers) is about 5 to about 25 nucleotides in length. In embodiments, each of the plurality of immobilized oligonucleotides (e.g., immobilized primers) is about 10 to about 40 nucleotides in length. In embodiments, each of the plurality of immobilized oligonucleotides (e.g., immobilized primers) is about 5 to about 100 nucleotides in length. In embodiments, each of the plurality of immobilized oligonucleotides (e.g., immobilized primers) is about 20 to 200 nucleotides in length. In embodiments, each of the plurality of immobilized oligonucleotides (e.g., immobilized primers) about or at least about 5, 6, 7, 8, 9, 10, 12, 15, 18, 20, 25, 30, 35, 40, 50 or more nucleotides in length. In embodiments, one or more immobilized oligonucleotides include blocking groups at their 3′ ends that prevent polymerase extension. A blocking moiety prevents formation of a covalent bond between the 3′ hydroxyl moiety of the nucleotide and the 5′ phosphate of another nucleotide. In embodiments, the 3′ modification is a 3′-phosphate modification, including a 3′ phosphate moiety, which is removed by a PNK enzyme or a phosphatase enzyme. Alternatively, abasic site cleavage with certain endonucleases (e.g., Endo IV) results in a 3′-OH at the cleavable site from the 3′-diesterase activity.

In embodiments, the immobilized oligonucleotides include one or more phosphorothioate nucleic acids. In embodiments, the immobilized oligonucleotides include a plurality of phosphorothioate nucleic acids. In embodiments, about or at least about 20%, 30%, 40%, 50%, 60%, 70%, 80%, 90%, or about 100% of the nucleotides in the immobilized oligonucleotides are phosphorothioate nucleic acids. In embodiments, most of the nucleotides in the immobilized oligonucleotides are phosphorothioate nucleic acids. In embodiments, all of the nucleotides in the immobilized oligonucleotides are phosphorothioate nucleic acids. In embodiments, none of the nucleotides in the immobilized oligonucleotides are phosphorothioate nucleic acids. In embodiments, the 5′ end of the immobilized oligonucleotide includes one or more phosphorothioate nucleic acids. In embodiments, the 5′ end of the immobilized oligonucleotide includes between one and five phosphorothioate nucleic acids.

In embodiments, the template nucleic acid (e.g., the template polynucleotide) is attached to the solid support (i.e., immobilized on the surface of a solid support). In embodiments, the complement of the template nucleic acid (e.g., the complement of the template polynucleotide) is attached to the solid support. The polynucleotide molecules can be fixed to surface by a variety of techniques, including covalent attachment and non-covalent attachment. In embodiments, the polynucleotides are confined to an area of a discrete region (referred to as a cluster). The discrete regions may have defined locations in a regular array, which may correspond to a rectilinear pattern, circular pattern, hexagonal pattern, or the like. A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. Interstitial regions will typically have a surface material that differs from the surface material of the features on the surface. For example, features of an array can have polynucleotides that exceeds the amount or concentration present at the interstitial regions. In some embodiments the polynucleotides and/or primers may not be present at the interstitial regions. In embodiments, at least two different primers are attached to the solid support (e.g., a forward and a reverse primer), which facilitates generating multiple amplification products from the first extension product or a complement thereof.

In embodiments of the methods and compositions provided herein, the clusters have a mean or median separation from one another of about 0.5-5 μm. In embodiments, the mean or median separation is about 0.1-10 microns, 0.25-5 microns, 0.5-2 microns, 1 micron, or a number or a range between any two of these values. In embodiments, the mean or median separation is about or at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 μm or a number or a range between any two of these values. In embodiments, the mean or median separation is about 0.1-10 microns. In embodiments, the mean or median separation is about 0.25-5 microns. In embodiments, the mean or median separation is about 0.5-2 microns. In embodiments, the mean or median separation is about or at least about 0.1 μm. In embodiments, the mean or median separation is about or at least about 0.25 μm. In embodiments, the mean or median separation is about or at least about 0.5 μm. In embodiments, the mean or median separation is about or at least about 1.0 μm. In embodiments, the mean or median separation is about or at least about 2.0 μm. In embodiments, the mean or median separation is about or at least about 5.0 μm. In embodiments, the mean or median separation is about or at least about 10 μm. The mean or median separation may be measured center-to-center (i.e., the center of one cluster to the center of a second cluster). In embodiments of the methods provided herein, the amplicon clusters have a mean or median separation (measured center-to-center) from one another of about 0.5-5 μm. The mean or median separation may be measured edge-to-edge (i.e., the edge of one amplicon cluster to the edge of a second amplicon cluster). In embodiments of the methods provided herein, the amplicon clusters have a mean or median separation (measured edge-to-edge) from one another of about 0.2-5 μm.

In embodiments of the methods provided herein, the amplicon clusters have a mean or median diameter of about 100-2000 nm, or about 200-1000 nm. In embodiments, the mean or median diameter is about 100-3000 nanometers, about 500-2500 nanometers, about 1000-2000 nanometers, or a number or a range between any two of these values. In embodiments, the mean or median diameter is about or at most about 100, 200, 300, 400, 500, 600, 700, 800, 900, 1,000, 1,100, 1,200, 1,300, 1,400, 1,500, 1,600, 1,700, 1,800, 1,900, 2000 nanometers or a number or a range between any two of these values. In embodiments, the mean or median diameter is about 100-3,000 nanometers. In embodiments, the mean or median diameter is about 100-2,000 nanometers. In embodiments, the mean or median diameter is about 500-2500 nanometers. In embodiments, the mean or median diameter is about 200-1000 nanometers. In embodiments, the mean or median diameter is about 1,000-2,000 nanometers. In embodiments, the mean or median diameter is about or at most about 100 nanometers. In embodiments, the mean or median diameter is about or at most about 200 nanometers. In embodiments, the mean or median diameter is about or at most about 500 nanometers. In embodiments, the mean or median diameter is about or at most about 400 nanometers. In embodiments, the mean or median diameter is about or at most about 500 nanometers. In embodiments, the mean or median diameter is about or at most about 600 nanometers. In embodiments, the mean or median diameter is about or at most about 700 nanometers. In embodiments, the mean or median diameter is about or at most about 1,000 nanometers. In embodiments, the mean or median diameter is about or at most about 2,000 nanometers. In embodiments, the mean or median diameter is about or at most about 2,500 nanometers. In embodiments, the mean or median diameter is about or at most about 3,000 nanometers.

In embodiments of the methods provided herein, each amplicon cluster (e.g., an amplicon cluster having a mean or median diameter of about 100-2000 nm, or about 200-1000 nm) includes about or at least about 100, 500, 1,000, 2,500, 5,000, 10,000, 15,000, 20,000, 25,000, 30,000, 35,000, 40,000, 45,000, or 50,000 dsDNA molecules. In embodiments, each amplicon cluster includes about 100 dsDNA molecules. In embodiments, each amplicon cluster includes about 500 dsDNA molecules. In embodiments, each amplicon cluster includes about 1000 dsDNA molecules. In embodiments, each amplicon cluster includes about 500 dsDNA molecules. In embodiments, each amplicon cluster includes about 1,000 dsDNA molecules. In embodiments, each amplicon cluster includes about 2,500 dsDNA molecules. In embodiments, each amplicon cluster includes about 5,000 dsDNA molecules. In embodiments, each amplicon cluster includes about 10,000 dsDNA molecules. In embodiments, each amplicon cluster includes about 20,000 dsDNA molecules. In embodiments, each amplicon cluster includes about 30,000 dsDNA molecules. In embodiments, each amplicon cluster includes about 40,000 dsDNA molecules. In embodiments, each amplicon cluster includes about 50,000 dsDNA molecules. In embodiments, each amplicon cluster includes more than about 50,000 dsDNA molecules.

In embodiments, the substrate is a particle. In embodiments, the substrate is a multiwell container. In embodiments, the substrate is a polymer coated particle or polymer coated planar support. In embodiments, the substrate includes a polymer. In embodiments, the particle includes polymerized units of polyacrylamide (AAm), poly-N-isopropylacrylamide, poly N-isopropylpolyacrylamide, sulfobetaine acrylate (SBA), carboxybetaine acrylate (CBA), phosphorylcholine acrylate (PCA), sulfobetaine methacrylate (SBMA), carboxybetaine methacrylate (CBMA), phosphorylcholine methacrylate (PCMA), polyethylene glycol acrylate, methacrylate, polyethylene glycol (PEG)-thiol/PEG-acrylate, acrylamide/N,N′-bis(acryloyl)cystamine (BACy), PEG/polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, glicydyl methacrylate (GMA), hydroxyethylmethacrylate (HEMA), hydroxyethylacrylate (HEA), hydroxypropylmethacrylate (HPMA), polyethylene glycol methacrylate (PEGMA), polyethylene glycol acrylate (PEGA), isocyanatoethyl methacrylate (IEM), or a copolymer thereof. In embodiments, the particle shell includes polymerized units of polyacrylamide (AAm), glicydyl methacrylate (GMA), polyethylene glycol methacrylate (PEGMA), polyethylene glycol methacrylate (PEGMA), isocyanatoethyl methacrylate (IEM), or a copolymer thereof. In embodiments, the particle includes polymerized units of polyethylene glycol methacrylate (PEGMA) and glicydyl methacrylate (GMA). In embodiments, the particle includes polymerized units of polyethylene glycol methacrylate (PEGMA) and isocyanatocthyl methacrylate (IEM). In embodiments, the particle includes polymerized units of 3-azido-2-hydroxypropyl methacrylate, 2-azido-3-hydroxypropyl methacrylate, 2-(((2-azidoethoxy)carbonyl)amino)ethyl methacrylate, 3-azido-2-hydroxypropyl acrylate, 2-azido-3-hydroxypropyl acrylate, or 2-(((2-azidocthoxy)carbonyl)amino)ethyl acrylate. In embodiments, the particle includes polymerized units of 3-azido-2-hydroxypropyl methacrylate, 2-azido-3-hydroxypropyl methacrylate, or 2-(((2-azidocthoxy)carbonyl)amino)ethyl methacrylate. In embodiments, the particle includes polymerized units of 3-azido-2-hydroxypropyl methacrylate. In embodiments, the particle includes polymerized units of 3-azido-2-hydroxypropyl methacrylate 2-azido-3-hydroxypropyl methacrylate. In embodiments, the particle includes polymerized units of 3-azido-2-hydroxypropyl methacrylate 2-(((2-azidocthoxy)carbonyl)amino)ethyl methacrylate.

In certain embodiments, presented herein is a kit for sequencing double stranded nucleic acid, in accordance with any of the methods described herein, and including one or more elements thereof. In embodiments, the kit includes: (i) a first adapter, wherein the first adapter includes a double-stranded portion and at least one single-stranded portion including one or more modified cytosine nucleobases; (ii) a second adapter, wherein the second adapter is a hairpin adapter including a nucleic acid having a 5′-end, a 5′-portion, a loop, a 3′-portion and a 3′-end, and the 5′-portion of the hairpin adapter is substantially complementary to the 3′-portion of the hairpin adapter; (iii) a first primer having a nucleic acid sequence complementary to a portion of the first adapter, or a complement thereof; and (iv) a second primer having a nucleic acid sequence complementary to the loop of the hairpin adapter, or a complement thereof. In certain embodiments, the first adapter is a Y-adapter, where the Y-adapter includes (i) a first strand having a 5′-portion and a 3′-portion, and (ii) a second strand having a 5′-portion and a 3′-portion, and the 3′-portion of the first strand is substantially complementary to the 5′-portion of the second strand, and the 5′-portion of the first strand is not substantially complementary to the 3′-portion of the second strand.

In embodiments, the kit includes at least a supply of a Y adapter as defined herein, a hairpin adapter, and a supply of at least one amplification primer which is capable of annealing to the Y adapter and priming synthesis of an extension product, and a supply of at least one amplification primer which is capable of annealing to the hairpin adapter and priming synthesis of an extension product.

The structure and properties of amplification primers will be well known to those skilled in the art. Suitable primers of appropriate nucleotide sequence for use with the adapters included in the kit can be readily prepared using standard automated nucleic acid synthesis equipment and reagents in routine use in the art. The kit may include as supply of one single type of primer or separate supplies (e.g., a mixture) of two different primers, for example a pair of PCR primers suitable for PCR amplification of templates modified with the adapters (e.g., Y adapter, hairpin adapter, or both adapters) in solution phase and/or on a suitable solid support (i.e. solid-phase PCR).

Adapters and/or primers may be supplied in the kits ready for use, or more preferably as concentrates-requiring dilution before use, or even in a lyophilized or dried form requiring reconstitution prior to use. If required, the kits may further include a supply of a suitable diluent for dilution or reconstitution of the primers. Optionally, the kits may further include supplies of reagents, buffers, enzymes, and dNTPs for use in carrying out nucleic acid amplification. Further components which may optionally be supplied in the kit include sequencing primers suitable for sequencing templates prepared using the methods described herein. In embodiments, the kit further includes instructions.

### III. Methods

In an aspect is provided a method of detecting methylation sites on a nucleic acid, the method including: (a) ligating a first adapter to a first end of the double stranded nucleic acid, and ligating a second adapter to a second end of the double stranded nucleic acid, wherein the second adapter is a hairpin adapter, thereby forming a nucleic acid template, the nucleic acid including a hairpin adapter portion, a double-stranded nucleic acid portion, and a Y adaptor portion, the double stranded nucleic acid portion including a first template single-stranded nucleic acid sequence hybridized to a second template single-stranded nucleic acid sequence; (b) annealing a primer to a sequence within the hairpin adapter portion; (c) extending the primer, thereby generating a first transcript nucleic acid (e.g., a blocking strand) hybridized to the first template single-stranded nucleic acid sequence, displacing the second template single-stranded nucleic acid sequence, and forming a template-transcript double stranded nucleic acid portion; and (d) detecting methylation sites on the displaced second template single stranded nucleic acid sequence.

In an aspect is provided a method of sequencing a nucleic acid including one or more cytosine nucleobases, the method including: (a) ligating a first adapter to a first end of the double stranded nucleic acid, and ligating a second adapter to a second end of the double stranded nucleic acid, wherein the second adapter is a hairpin adapter, thereby forming a nucleic acid template, wherein the nucleic acid template includes a hairpin adapter portion, a double-stranded nucleic acid portion, and a first adaptor portion, wherein the double stranded nucleic acid portion includes a first template single-stranded nucleic acid sequence hybridized to a second template single-stranded nucleic acid sequence; (b) annealing a blocking primer to a sequence of the nucleic acid template and extending the blocking primer, thereby generating a blocking strand hybridized to the first template single-stranded nucleic acid sequence, displacing the second template single-stranded nucleic acid sequence, and forming a template-blocking double stranded nucleic acid portion; (c) converting the one or more cytosine nucleobases of the second template strand to a uracil nucleobase, or uracil nucleobase analog; and (d) sequencing the second template strand by annealing a sequencing primer to the nucleic acid template and extending the sequencing primer, thereby sequencing a nucleic acid including one or more cytosine nucleobases.

In an aspect is provided a method of sequencing a nucleic acid molecule, wherein the nucleic acid molecule includes, from 5′ to 3′, a first strand, a first primer binding sequence, a second strand including a cytosine nucleobase, and a second primer binding sequence, wherein the second strand is complementary to the first strand, the method including: (a) annealing a blocking primer to the first primer binding sequence of the nucleic acid molecule and extending the blocking primer with a polymerase to form a blocking strand hybridized to the first strand; (b) converting the cytosine nucleobase of the second strand to a uracil nucleobase, or uracil nucleobase analog; and (c) sequencing the second strand to generate a sequencing read.

In embodiments, the one or more cytosine nucleobases of the second template strand include modified cytosine nucleobases. In embodiments, the modified cytosine nucleobases of the second template strand include 5-methylcytosine (5mC), 5-hydroxymethyl cytosine (5hmC), 5-formylcytosine (5fC), 5-carboxylcytosine (5caC), or β-glucosyl-5-hydroxymethylcytosine (5gmC). In embodiments, the modified cytosine nucleobases include 5-methylcytosine (5mC) or 5-hydroxymethyl cytosine (5hmC). In embodiments, the modified cytosine nucleobases of the second template strand include 5-methylcytosine (5mC). In embodiments, the modified cytosine nucleobases of the second template strand include 5-hydroxymethyl cytosine (5hmC). In embodiments, the modified cytosine nucleobases of the second template strand include 5-formylcytosine (5fC). In embodiments, the modified cytosine nucleobases of the second template strand include 5-carboxylcytosine (5caC). In embodiments, the modified cytosine nucleobases of the second template strand include β-glucosyl-5-hydroxymethylcytosine (5gmC). In embodiments, the modified cytosine nucleobases include 5-methylcytosine (5mC) or 5-hydroxymethyl cytosine (5hmC). In embodiments, the one or more cytosine nucleobases of the second template strand include unmodified cytosine nucleobases (e.g., native cytosine nucleobases). In embodiments, the one or more cytosine nucleobases of the second template strand include modified and unmodified cytosine nucleobases.

In embodiments, the cytosine nucleobase of the second strand that is converted to the uracil nucleobase, or uracil nucleobase analog, is a modified cytosine nucleobase. In embodiments, the modified cytosine nucleobase of the second strand is a 5-methylcytosine (5mC), 5-hydroxymethyl cytosine (5hmC), 5-formylcytosine (5fC), 5-carboxylcytosine (5caC), or β-glucosyl-5-hydroxymethylcytosine (5gmC). In embodiments, the modified cytosine nucleobase is a 5-methylcytosine (5mC) or 5-hydroxymethyl cytosine (5hmC). In embodiments, the modified cytosine nucleobase of the second strand is a 5-methylcytosine (5mC). In embodiments, the modified cytosine nucleobase of the second strand is a 5-hydroxymethyl cytosine (5hmC). In embodiments, the modified cytosine nucleobase of the second strand is a 5-formylcytosine (5fC). In embodiments, the modified cytosine nucleobase of the second strand is a 5-carboxylcytosine (5caC). In embodiments, the modified cytosine nucleobase of the second strand is a β-glucosyl-5-hydroxymethylcytosine (5gmC). In embodiments, the modified cytosine nucleobase is a 5-methylcytosine (5mC) or 5-hydroxymethyl cytosine (5hmC). In embodiments, the cytosine nucleobase of the second strand is an unmodified cytosine nucleobase (e.g., native cytosine nucleobase).

In some embodiments, a double stranded nucleic acid (i.e., a duplex) includes two complementary nucleic acid strands. In embodiments, a double stranded nucleic acid includes a first strand and a second strand which are complementary or substantially complementary to each other. A first strand of a double stranded nucleic acid is sometimes referred to herein as a forward strand and a second strand of the double stranded nucleic acid is sometime referred to herein as a reverse strand. In some embodiments, a double stranded nucleic acid includes two opposing ends. Accordingly, a double stranded nucleic acid often includes a first end and a second end. An end of a double stranded nucleic acid may include a 5′-overhang, a 3′-overhang or a blunt end. In some embodiments, one or both ends of a double stranded nucleic acid are blunt ends. In certain embodiments, one or both ends of a double stranded nucleic acid are manipulated to include a 5′-overhang, a 3′-overhang or a blunt end using a suitable method. In some embodiments, one or both ends of a double stranded nucleic acid are manipulated during library preparation such that one or both ends of the double stranded nucleic acid are configured for ligation to an adapter using a suitable method. For example, one or both ends of a double stranded nucleic acid may be digested by a restriction enzyme, polished, end-repaired, filled in, phosphorylated (e.g., by adding a 5′-phosphate), dT-tailed, dA-tailed, the like or a combination thereof.

In embodiments, the double stranded nucleic acid, alternatively referred to as a library insert or target polynucleotide, is at least 50, 100, 150, 200, 250, or 300 nucleotides in length. In embodiments, the double stranded nucleic acid, alternatively referred to as a library insert, is at least 150, 200, 250, 300, 350, or 400 nucleotides in length. In embodiments, the double stranded nucleic acid, alternatively referred to as a library insert, is at least 450, 500, 650, 700, 750, or 800 nucleotides in length. In embodiments, the double stranded nucleic acid, alternatively referred to as a library insert, is at least 850, 900, 950, 1000, 1050, or 1100 nucleotides in length.

In embodiments, the double stranded nucleic acid, alternatively referred to as a library insert, is about 50, 100, 150, 200, 250, or 300 nucleotides in length. In embodiments, the double stranded nucleic acid, alternatively referred to as a library insert, is about 150, 200, 250, 300, 350, or 400 nucleotides in length. In embodiments, the double stranded nucleic acid, alternatively referred to as a library insert, is about 450, 500, 650, 700, 750, or 800 nucleotides in length. In embodiments, the double stranded nucleic acid, alternatively referred to as a library insert, is about 850, 900, 950, 1000, 1050, or 1100 nucleotides in length. In embodiments, the double stranded nucleic acid, alternatively referred to as a library insert, is about 500-1500 nucleotides in length. In embodiments, the double stranded nucleic acid, alternatively referred to as a library insert, is about 750-1500 nucleotides in length. In embodiments, the double stranded nucleic acid, alternatively referred to as a library insert, is about 1-2 kilobases (kb) in length. In embodiments, the double stranded nucleic acid, alternatively referred to as a library insert, is about 300, 400, 600, or 800 nucleotides in length. In embodiments, the double stranded nucleic acid, alternatively referred to as a library insert, is about 250 to 600 nucleotides in length.

In embodiments, the double stranded nucleic acid is about 100, 125, 150, 175, or 200 nucleotides in length. In embodiments, the double stranded nucleic acid is about 200, 225, 250, 275, or 300 nucleotides in length. In embodiments, the double stranded nucleic acid is less than 150 nucleotides in length. In embodiments, the double stranded nucleic acid is less than 100 nucleotides in length. In embodiments, the double stranded nucleic acid is less than 75 nucleotides in length. In embodiments, the double stranded nucleic acid is about 150 nucleotides in length. In embodiments, the double stranded nucleic acid is about 100 nucleotides in length. In embodiments, the double stranded nucleic acid is about 75 nucleotides in length. In embodiments, the method provides sequencing both strands of a double stranded nucleic acid such that there is overlap in the sequencing reads of the first and second strand. For example, if the double stranded nucleic acid is short (e.g., 150-200 nucleotides) it is possible to sequence the first strand and a complementary region of the second strand (e.g., in the same read).

In embodiments, the double stranded nucleic acid is greater than 150 nucleotides in length. In embodiments, the double stranded nucleic acid is greater than 200 nucleotides in length. In embodiments, the double stranded nucleic acid is greater than 250 nucleotides in length. In embodiments, the double stranded nucleic acid is greater than 300 nucleotides in length. In embodiments, the double stranded nucleic acid is greater than 500 nucleotides in length. In embodiments, the double stranded nucleic acid is greater than 700 nucleotides in length. In embodiments, the double stranded nucleic acid is greater than 900 nucleotides in length. In embodiments, the double stranded nucleic acid is greater than 1,000 nucleotides in length (i.e., greater than 1 kb). In embodiments, the method provides sequencing both strands of a double stranded nucleic acid such that there is no overlap in the sequencing reads of the first and second strand, rather a portion of the first strand and portion of the second strand.

In some embodiments, the methods described herein includes ligating one or more adapters to a double stranded nucleic acid. In some embodiments, the methods described herein includes ligating one or more adapters to a plurality of double stranded nucleic acids. In some embodiments, the methods described herein includes ligating a first adapter to a first end of a double stranded nucleic acid, and ligating a second adapter to a second end of a double stranded nucleic acid. In some embodiments, the first adapter and the second adapter are different (e.g., non-identical adapters). For example, in certain embodiments, the first adapter and the second adapter may include different nucleic acid sequences or different structures. In some embodiments, the first adapter is a Y-adapter and the second adapter is a hairpin adapter. In some embodiments, the first adapter is a hairpin adapter and a second adapter is a hairpin adapter. In certain embodiments, the first adapter and the second adapter may include different primer binding sites, different structures, and/or different capture sequences (e.g., a sequence complementary to a capture nucleic acid). In some embodiments, some, all or substantially all of the nucleic acid sequence of a first adapter and a second adapter are the same. In some embodiments, some, all or substantially all of the nucleic acid sequence of a first adapter and a second adapter are substantially different.

In embodiments, the double-stranded nucleic acid is a DNA sample. In embodiments, the DNA sample includes genomic DNA. In embodiments, the DNA sample includes picogram quantities of DNA. In embodiments, the DNA sample includes about 1 pg to about 900 pg DNA, about 1 μg to about 500 pg DNA, about 1 μg to about 100 pg DNA, about 1 μg to about 50 pg DNA, about 1 to about 10 pg, DNA, less than about 200 pg, less than about 100 pg DNA, less than about 50 pg DNA, less than about 20 pg DNA, and less than about 5 pg DNA. In other embodiments, the DNA sample includes nanogram quantities of DNA. In embodiments, the DNA sample contains about 1 to about 500 ng of DNA, about 1 to about 200 ng of DNA, about 1 to about 100 ng of DNA, about 1 to about 50 ng of DNA, about 1 ng to about 10 ng of DNA, about 1 ng to about 5 ng of DNA, less than about 100 ng of DNA, less than about 50 ng of DNA less than about 5 ng of DNA, or less that about 2 ng of DNA. In embodiments, the DNA sample includes circulating cell-free DNA (cfDNA). In embodiments, the DNA sample includes microgram quantities of DNA.

In embodiments, the double-stranded nucleic acid includes one or more 5-methylcytosine (5mC) or 5-hydroxymethyl cytosine (5hmC) nucleobases. In embodiments, the double-stranded nucleic acid includes one or more 5-methylcytosine (5mC), 5-hydroxymethyl cytosine (5hmC), 5-formylcytosine (5fC), 5-carboxylcytosine (5caC), or β-glucosyl-5-hydroxymethylcytosine (5gmC) nucleobases. In embodiments, the double-stranded nucleic acid includes one or more 5-methylcytosine (5mC) nucleobases. In embodiments, the double-stranded nucleic acid includes one or more 5-hydroxymethyl cytosine (5hmC) nucleobases. In embodiments, the double-stranded nucleic acid includes one or more 5-formylcytosine (5fC) nucleobases. In embodiments, the double-stranded nucleic acid includes one or more 5-carboxylcytosine (5caC) nucleobases. In embodiments, the double-stranded nucleic acid includes one or more β-glucosyl-5-hydroxymethylcytosine (5gmC) nucleobases.

In embodiments, the double-stranded nucleic acid includes a 5-methylcytosine (5mC) or 5-hydroxymethyl cytosine (5hmC) nucleobase. In embodiments, the double-stranded nucleic acid includes a 5-methylcytosine (5mC), 5-hydroxymethyl cytosine (5hmC), 5-formylcytosine (5fC), 5-carboxylcytosine (5caC), or β-glucosyl-5-hydroxymethylcytosine (5gmC) nucleobase. In embodiments, the double-stranded nucleic acid includes a 5-methylcytosine (5mC) nucleobase. In embodiments, the double-stranded nucleic acid includes a 5-hydroxymethyl cytosine (5hmC) nucleobase. In embodiments, the double-stranded nucleic acid includes a 5-formylcytosine (5fC) nucleobase. In embodiments, the double-stranded nucleic acid includes a 5-carboxylcytosine (5caC) nucleobase. In embodiments, the double-stranded nucleic acid includes a β-glucosyl-5-hydroxymethylcytosine (5gmC) nucleobase.

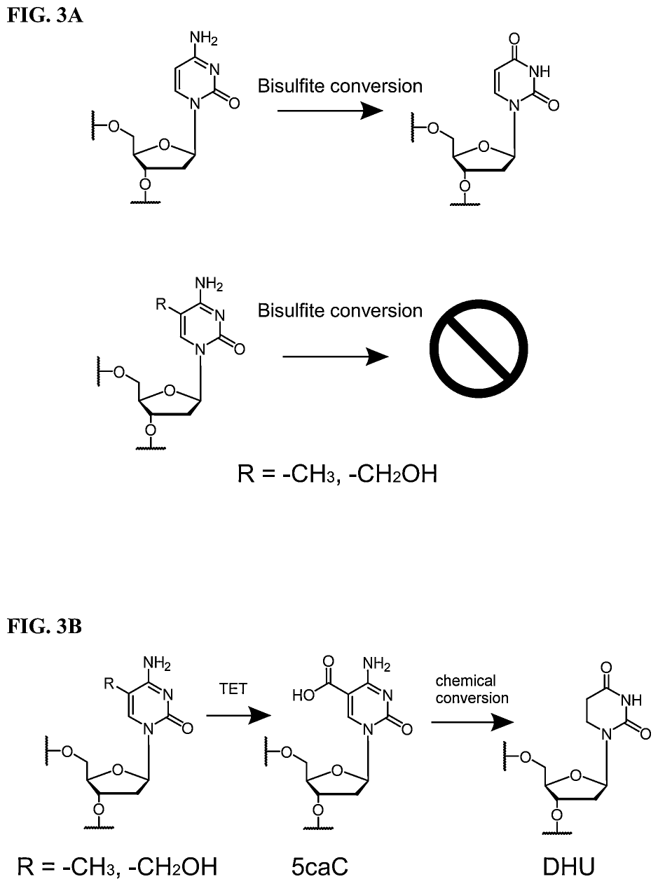

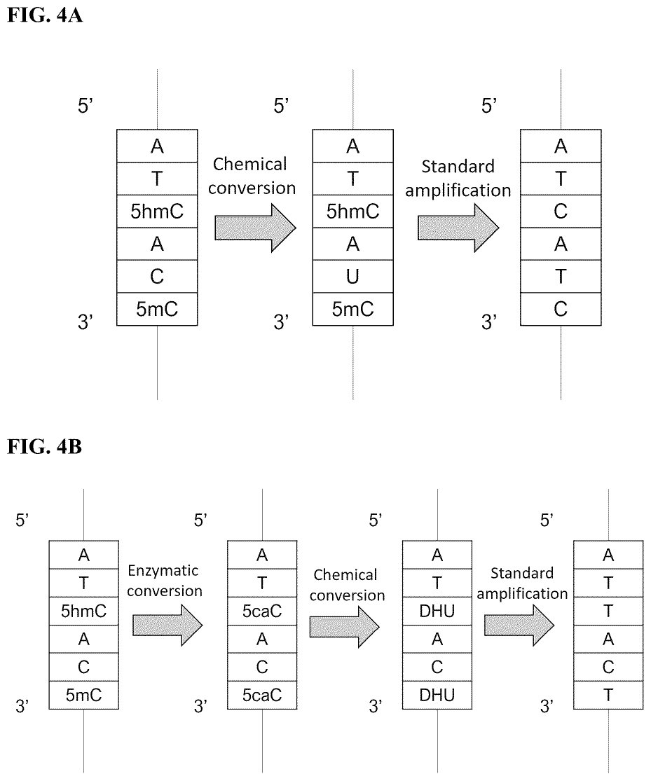

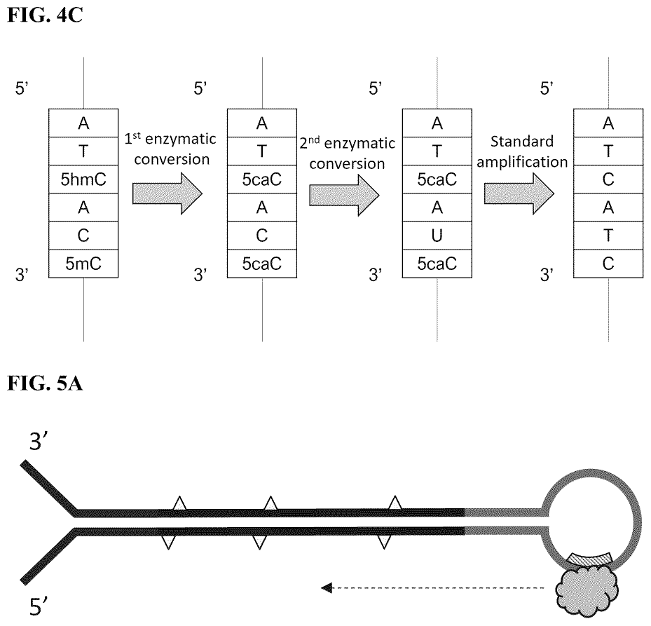

Conversion approaches: chemical approaches. Chemical approaches for converting methylated cytosines have been known for decades. A commonly used agent for modifying unmethylated cytosine preferentially to methylated cytosine is sodium bisulfite. Sodium bisulfite (NaHSO₃) reacts readily with the 5,6-double bond of cytosine, but poorly with methylated cytosine, as described by Olek A., Nucleic Acids Res. 24:5064-6, 1996 or Frommer et al., Proc. Natl. Acad. Sci. USA 89:1827-1831 (1992), each of which is incorporated herein by reference. Cytosine reacts with the bisulfite ion to form a sulfonated cytosine reaction intermediate which is susceptible to deamination, giving rise to a sulfonated uracil. The sulfonate group can be removed under alkaline conditions, resulting in the formation of uracil nucleobase (see FIG. 3A and FIG. 4A for additional detail). Alternatively, conversion may be accomplished using restriction enzymes, such as HpaII and MspI, which recognize the sequence CCGG. Uracil is recognized as a thymine by Taq polymerase and other polymerases and therefore upon amplification (e.g., PCR) and subsequently during detection (e.g., sequencing reaction), the resultant product contains cytosine only at the position where 5-methylcytosine occurs in the initial template nucleic acid.

In embodiments, the one or more cytosine nucleobases of the second strand that are converted to the uracil nucleobase, or uracil nucleobase analog, are unmodified cytosine nucleobases. In embodiments, converting the one or more cytosine nucleobases (e.g., one or more unmodified cytosine nucleobases) of the second template strand includes i) contacting the one or more cytosine nucleobases (e.g., one or more unmodified cytosine nucleobases) with sodium bisulfite to generate one or more uracil nucleobases.

In embodiments, the cytosine nucleobase of the second strand that is converted to the uracil nucleobase, or uracil nucleobase analog, is an unmodified cytosine nucleobase. In embodiments, converting the unmodified cytosine nucleobase of the second strand includes i) contacting the unmodified cytosine nucleobase with sodium bisulfite to generate a uracil nucleobase.

Conversion approaches: enzymatic approaches. A method for bisulfite-free direct detection of 5-methylcytosine (5mC) and 5-hydroxymethylcytosinc (5hmC) has been described (Liu Y et al. Nat. Biotechnol. 2019, 37(4)424-429, which is incorporated herein by reference), which combines ten-eleven translocation (TET) enzymatic oxidation of 5mC and 5hmC to 5-carboxylcytosine (5caC) with pyridine borane reduction of 5caC to dihydrouracil (DHU). Another bisulfite-free approach for methylation analysis is the NEBNext® Enzymatic Methyl-seq product, which first protects 5mC and 5hmC from deamination by TET2 and an oxidation enhancer, followed by APOBEC deamination of unprotected cytosines to uracils.

In another aspect is provided a method of detecting a single-nucleotide polymorphism (SNP) or a single-nucleotide variant (SNV) in a double-stranded nucleic acid. One unresolved problem with bisulfite conversion is that it is often difficult to distinguish between SNPs or unmethylated cytosine in the same double stranded nucleic acid. This is especially difficult for C>T SNPs, which is the most common substitution (>60%) in human population. Often this requires very high sequencing depth to discern simultaneous SNVs, SNPs, and methylation profiles. For example, computational methods have been developed to predict germline SNPs in bulk sequencing and suggest that at least 30× genomic coverage is required to identify 96% of SNPs from bisulfite converted DNA. Using methods described herein, e.g., generating the Y-template-hairpin constructs, and obtaining sequencing information from both strands permits identification of a SNP and a methylation profile. In embodiments, the methods described herein reduce sequencing overhead with higher accuracy for ctDNA. Methods described herein also differentiate SNV and methylation simultaneously, at low sequencing depths for germline mutations.

In embodiments, the method includes detecting SNVs and methylation status from a double stranded nucleic acid. Although SNVs can determine if a mutation occurred, it cannot reveal tissue of origin. Methylation is highly tissue specific and can be used to predict tissue of origin of cfDNA. Current studies have shown common methylation CpG sites that are differentially methylated depending on tissue. By searching for these different methylation signals within ctDNA, one could determine if there are elevated levels of certain tissue signals within the plasma.

Alternatively, a first modified cytosine nucleobase, such as 5mC or 5hmC, may be enzymatically converted to a second modified nucleobase, such as 5caC utilizing a TET enzyme. In embodiments, the second modified nucleobase, 5caC, may be further converted to dihydrouracil (DHU) following contact with a borane-agent (e.g., pyridine borane). In embodiments, the cytosine nucleobases include unmodified cytosine, 5mC, 5gmC, 5hmC nucleobases, or a combination thereof. In embodiments, the cytosine nucleobases include 5mC, 5gmC, or 5hmC nucleobases. In embodiments, the cytosine nucleobases include 5mC or 5hmC nucleobases. In embodiments, the cytosine nucleobases include 5mC and 5hmC nucleobases.

In embodiments, converting the one or more cytosine nucleobases of the second template strand includes contacting the one or more cytosine nucleobases with a ten-eleven translocation (TET) enzyme, an apolipoprotein B mRNA editing enzyme, catalytic polypeptide-like (APOBEC) enzyme, a borane-containing reducing agent, an oxidizing agent, or a combination thereof. In embodiments, the cytosine nucleobases include unmodified cytosine, 5mC, 5gmC, 5hmC nucleobases, or a combination thereof. In embodiments, converting the cytosine nucleobase of the second strand includes contacting the cytosine nucleobase with a ten-eleven translocation (TET) enzyme, an apolipoprotein B mRNA editing enzyme, catalytic polypeptide-like (APOBEC) enzyme, a borane-containing reducing agent, an oxidizing agent, or a combination thereof.

In embodiments, converting the one or more cytosine nucleobases of the second template strand includes i) contacting the one or more cytosine nucleobases with a ten-eleven translocation (TET) enzyme to generate one or more 5-carboxylcytosine (5caC) nucleobases; and ii) contacting the one or more 5caC nucleobases with borane-containing reducing agent to generate one or more uracil nucleobase analogs. In embodiments, converting the cytosine nucleobase of the second strand includes i) contacting the cytosine nucleobase with a ten-eleven translocation (TET) enzyme to generate a 5-carboxylcytosine (5caC) nucleobase; and ii) contacting the 5caC nucleobase with a borane-containing reducing agent to generate a uracil nucleobase analog.

In embodiments, converting the one or more cytosine nucleobases of the second template strand includes i) contacting the one or more cytosine nucleobases with a β-glucosyltransferase to generate one or more β-glucosyl-5-hydroxymethylcytosine (5gmC) nucleobases; ii) contacting the one or more 5gmC nucleobases with a ten-eleven translocation (TET) enzyme to generate one or more 5-carboxylcytosine (5caC) nucleobases; and iii) contacting the one or more 5caC nucleobases with borane-containing reducing agent to generate one or more uracil nucleobase analogs. In embodiments, converting the cytosine nucleobase of the second strand includes i) contacting the cytosine nucleobase with a β-glucosyltransferase to generate a β-glucosyl-5-hydroxymethylcytosine (5gmC) nucleobase; ii) contacting the 5gmC nucleobase with a ten-eleven translocation (TET) enzyme to generate a 5-carboxylcytosine (5caC) nucleobase; and iii) contacting the 5caC nucleobase with a borane-containing reducing agent to generate a uracil nucleobase analog.

In embodiments, converting the one or more cytosine nucleobases of the second template strand includes i) contacting the one or more cytosine nucleobases with an oxidizing agent to generate one or more 5-formyl cytosine (5fC) nucleobases; and ii) contacting the one or more 5caC nucleobases with borane-containing reducing agent to generate one or more uracil nucleobase analogs, wherein the oxidizing agent is selected from the group consisting of potassium perruthenate (KRuO₄), Cu(II)/TEMPO (copper(II) perchlorate and 2,2,6,6-tetramethylpiperidine-1-oxyl (TEMPO)), potassium ruthenate, and manganese oxide. In embodiments, converting the cytosine nucleobase of the second strand includes i) contacting the cytosine nucleobase with an oxidizing agent to generate a 5-formyl cytosine (5fC) nucleobase; and ii) contacting the 5caC nucleobase with a borane-containing reducing agent to generate a uracil nucleobase analog, wherein the oxidizing agent is selected from the group consisting of potassium perruthenate (KRu04), Cu(II)/TEMPO (copper(II) perchlorate and 2,2,6,6-tetramethylpiperidine-1-oxyl (TEMPO)), potassium ruthenate, and manganese oxide. Additional examples of using the oxidizing agent(s) to generate one or more 5-formyl cytosine (5fC) nucleobases may be found, for example, in U.S. Pat. Pub. 2018/0251815, which is incorporated herein by reference in its entirety.

In an embodiment, converting the one or more cytosine nucleobases of the second template strand includes i) contacting the one or more cytosine nucleobases with a ten-eleven translocation (TET) enzyme to generate one or more 5-carboxylcytosine (5caC) nucleobases; and ii) contacting the second template strand with an apolipoprotein B mRNA editing enzyme, catalytic polypeptide-like (APOBEC) enzyme to generate one or more uracil nucleobases. In embodiments, converting the cytosine nucleobase of the second template strand includes i) contacting the cytosine nucleobase with a ten-eleven translocation (TET) enzyme to generate a 5-carboxylcytosine (5caC) nucleobase; and ii) contacting the second template strand with an apolipoprotein B mRNA editing enzyme, catalytic polypeptide-like (APOBEC) enzyme to generate a uracil nucleobase.

In embodiments, the step of converting the 5caC and/or 5fC to DHU includes contacting the DNA sample with a reducing agent including, for example, pyridine borane, 2-picoline borane (pic-BEb), tert-butyl amine borane, borane, sodium borohydride, sodium cyanoborohydride, and sodium triacetoxyborohydride. In embodiments, the reducing agent is pic-BEb and/or pyridine borane.

In an embodiment, extending the blocking primer includes extending the blocking primer with a strand-displacing polymerase. In embodiments, the polymerase is a strand-displacing polymerase. In embodiments, generating the blocking strand includes a plurality of blocking primer extension cycles. In embodiments, generating the blocking strand includes extending the blocking primer by incorporating one or more nucleotides (e.g., dNTPs) using Bst large fragment (Bst LF) polymerase, Bst2.0 polymerase, Bsu polymerase, SD polymerase, Vent exo-polymerase, Phi29 polymerase, or a mutant thereof.

In embodiments, the step of determining the sequence of the modified target DNA includes chain termination sequencing, microarray, high-throughput sequencing, and restriction enzyme analysis. In embodiments, the step of detecting the sequence of the modified target DNA includes a next generation sequencing method.

In an embodiment, the method further includes sequencing the first template strand by annealing a second sequencing primer to the nucleic acid template and extending the sequencing primer.

In embodiments, sequencing includes sequencing by synthesis. In some embodiments, methods provided herein include sequencing a template nucleic acid or amplicon described herein. The methods of template preparation and nucleic acid sequencing described herein can be incorporated into a suitable sequencing technique, non-limiting examples of which include SMRT (single-molecule real-time sequencing), ion semiconductor, pyrosequencing, sequencing by synthesis, combinatorial probe anchor synthesis, and SOLiD sequencing (sequencing by ligation). Non-limiting sequencing platforms include those provided by Singular Genomics™ (e.g., the G4™ sequencing platform), Illumina® (e.g., the MiniSeq™, MiSeq™, NextSeq™, and/or NovaSeq™ sequencing systems); Ion Torrent™ (e.g., the Ion PGM™, Ion S5™, and/or Ion Proton™ sequencing systems); Pacific Biosciences (e.g., the PACBIO RS II and/or Sequel II System sequencing system); ThermoFisher (e.g., a SOLID® sequencing system); or BGI Genomics (e.g., DNBSeq™ sequencing systems). See, for example U.S. Pat. Nos. 7,211,390; 7,244,559; 7,264,929; 6,255,475; 6,013,445; 8,882,980; U.S. Pat. Nos. 6,664,079; and 9,416,409. In some embodiments, a sequencing method described herein does not include the use of SMRT sequencing or single-molecule sequencing.

In embodiments, the method includes sequencing the first and the second strand of a double-stranded template and/or amplification product by extending a sequencing primer hybridized thereto. A variety of sequencing methodologies can be used such as sequencing-by-synthesis (SBS), pyrosequencing, sequencing-by-binding (SBB), sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. No. 10,738,072 and Chen et al, Proteomics & Bioinformatics, V. 11, Issue 1, 2013, Pages 34-40, each of which are incorporated herein by reference. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

Sequencing includes, for example, detecting a sequence of signals. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. A variety of sequencing chemistries are available, non-limiting examples of which are described herein.

In embodiments, sequencing is performed according to a “sequencing-by-binding” method (see, e.g., U.S. Pat. Pubs. US2017/0022553 and US2019/0048404, each of which is incorporated herein by reference in its entirety), which refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

In embodiments, sequencing includes hybridizing a sequencing primer to the second primer binding sequence, incorporating one or more modified nucleotides into the sequencing primer with a polymerase to create an extension strand, and detecting the one or more incorporated nucleotides. In embodiments, sequencing includes hybridizing a sequencing primer to the first primer binding sequence, incorporating one or more modified nucleotides into the sequencing primer with a polymerase to create an extension strand, and detecting the one or more incorporated nucleotides.

In embodiments, sequencing includes annealing a sequencing primer to the second primer binding sequence and contacting the sequencing primer with a sequencing solution including one or more modified nucleotides including a reversible terminator, and monitoring the sequential incorporation of complementary nucleotides to generate one or more sequencing reads, wherein the reversible terminator is removed prior to the introduction of the next complementary nucleotide. In embodiments, sequencing includes annealing a sequencing primer to the first primer binding sequence and contacting the sequencing primer with a sequencing solution including one or more modified nucleotides including a reversible terminator, and monitoring the sequential incorporation of complementary nucleotides to generate one or more sequencing reads, wherein the reversible terminator is removed prior to the introduction of the next complementary nucleotide.

In an aspect is provided a method of generating a double-stranded nucleic acid including one or more cytosine mismatches, the method including: (a) ligating a first adapter to a first end of the double-stranded nucleic acid, ligating a second adapter to a second end of the double stranded nucleic acid, wherein the second adapter is a hairpin adapter, thereby forming a nucleic acid template, wherein the nucleic acid template includes a hairpin adapter portion, a double-stranded nucleic acid portion, and a first adapter portion, wherein the double stranded nucleic acid portion includes one or more cytosine nucleobases and a first template single-stranded nucleic acid sequence hybridized to a second template single-stranded nucleic acid sequence; (b) annealing a blocking primer to a sequence of the nucleic acid template and extending the blocking primer, thereby generating a blocking strand hybridized to the first template single-stranded nucleic acid sequence, displacing the second template single-stranded nucleic acid sequence, and forming a template-blocking double stranded nucleic acid portion; (c) converting the one or more cytosine nucleobases of the second template strand to a uracil nucleobase, or uracil nucleobase analog; thereby forming the double-stranded nucleic acid including one or more cytosine mismatches.

In an aspect is provided a method of generating a double-stranded nucleic acid including a cytosine mismatch, the method including: (a) ligating a first hairpin adapter to a first end of the double-stranded nucleic acid molecule, and ligating a second adapter to a second end of the double-stranded nucleic acid, thereby forming a nucleic acid template, wherein the double-stranded nucleic acid includes a first strand hybridized to a second strand, and wherein the second strand includes a cytosine nucleobase; (b) annealing a blocking primer to a sequence of the nucleic acid template and extending the blocking primer, thereby generating a blocking strand hybridized to the first strand of the double-stranded nucleic acid, and displacing the second strand of the double-stranded nucleic acid; (c) converting the cytosine nucleobase of the displaced second strand to a uracil nucleobase, or uracil nucleobase analog, to form a converted second strand; (d) removing the blocking strand and hybridizing the first strand to the converted second strand, thereby forming the double-stranded nucleic acid including a cytosine mismatch.

In embodiments, the blocking primer includes one or more locked nucleic acids (LNAs), 2-amino-deoxyadenosine (2-amino-dA), trimethoxystilbene-functionalized oligonucleotides (TFOs), Pyrene-functionalized oligonucleotides (PFOs), peptide nucleic acids (PNAs), or aminoethyl-phenoxazine-dC (AP-dC) nucleotides. In embodiments, the blocking primer includes one or more minor groove binder (MGB), C-5 propynyl-deoxycytidine, C-5 propynyl-deoxyuridine, aminoethyl-phenoxazine-deoxycitidine, 5-methyl-deoxycitidine, or 2-amino-deoxyadenosine. In embodiments, the trimethoxystilbene-functionalized oligonucleotides (TFOs) are included at the 5′ end of the blocking primer, and increase the Tm by about 10° C. Including a trimethoxystilbene modification enables manufacturing of hybridization probes with greater affinity to complementary sequences. In embodiments, the pyrene-functionalized oligonucleotides may be generated by a) replacing the nucleobase moiety, b) through attachment to the nucleobase moiety, and c) through attachment to the sugar moiety, as described in Ostergaard M E and Hrdlicka P J. Chem. Soc. Rev. 2011; 40(12): 5771-5788, which is incorporated herein by reference. Pyrenes function as polarity-sensitive and quenchable fluorophores, excimer-generating units, aromatic stacking moieties, and nucleic acid duplex intercalators. The attachment point and linker length influence the position and characteristics of the pyrene label within a nucleic acid duplex. Pyrene-functionalized LNAs display distinctive properties such as large fluorescence quantum yields, efficient discrimination of SNPs, and recognition of mixed-sequence double stranded DNA.

In embodiments, the method further includes amplifying the nucleic acid template including one or more cytosine mismatches to generate amplicons including one or more cytosine mismatches.

In embodiments, the method further includes amplifying the nucleic acid template including the cytosine mismatch to generate amplicons including the cytosine mismatch.

In embodiments, the method further includes annealing a probe oligonucleotide to the second template single-stranded nucleic acid sequence and separating the probe-hybridized double-stranded nucleic acid from nucleic acids not hybridized to a probe. In embodiments, the method further includes annealing a probe oligonucleotide to the displaced second strand and separating the probe-hybridized double-stranded nucleic acid from nucleic acids not hybridized to a probe. In embodiments, the probe oligonucleotide contains a sequence capable of hybridizing to a mutated sequence (i.e., a hotspot sequence) as identified in Catalogue of Somatic Mutations In Cancer (COSMIC), full-length genes, copy number genes, single nucleotide polymorphisms (SNPs), or inter- and intragenic gene fusions. In embodiments, the probe oligonucleotide contains a sequence capable of hybridizing to a region of interest, such as a gene associated with cancer (e.g., lung, colon, breast, ovarian, melanoma, or prostate cancer) see for example Simen B B, Arch Pathol Lab Med; 139(4):508-517 (2015) or Singh R R, J Mol Diagn. September; 15(5):607-22 (2013); a gene associated with a disease (e.g., retinopathy, epilepsy, immunodeficiency, cardiomyopathy, hearing loss, muscular dystrophy, aneuploidy), see for example S. Yohe et al. Vol. 139, No. 2, pp. 204-210 (2015) or Rehm H L. Nat Rev Genet. 14(4):295-300 (2013); or a gene associated with persisting pain (see for example Kringel et al. Front. Pharmacol. V9 Art. 1008 2018).

In embodiments, the probe oligonucleotide contains a sequence capable of hybridizing to uracil nucleobases (e.g., following conversion of cytosine nucleobases with a conversion agent) of the second template single-stranded nucleic acid sequence and separating the probe-hybridized double-stranded nucleic acid from nucleic acids not hybridized to a probe. For example, the probe oligonucleotide contains a dA nucleobase that complements the dU nucleobase that is expected to result from bisulfite conversion of each of the dC positions in a template single-stranded nucleic acid sequence (e.g., the converted dC nucleobase of a CpG site). The probe oligonucleotide may alternatively contain a dG nucleobase that complements the dC nucleobase of a CpG site in a template single-stranded nucleic acid sequence (e.g., a methylated dC nucleobase or non-methylated dC nucleobase of a CpG site). Additional probe oligonucleotide exemplary designs for targeted methylation sequencing may be found, for example, in U.S. Pat. Pub. 2017/0175205, which is incorporated herein by reference in its entirety. In embodiments, following converting the one or more cytosine nucleobases of the second template strand to a uracil nucleobase, or uracil nucleobase analog, the method further includes annealing a probe oligonucleotide to the uracil-containing strand, wherein the probe oligonucleotide is capable of hybridizing to the uracil-containing sequence (e.g., the probe oligonucleotide contains one of more dA nucleobases that complement the one or more dU nucleobases). In embodiments, following converting the one or more cytosine nucleobases of the second template strand to a uracil nucleobase, or uracil nucleobase, the method further includes annealing a probe oligonucleotide to the methylated cytosine-containing strand, wherein the probe oligonucleotide is capable of hybridizing to the methylated cytosine-containing sequence (e.g., the probe oligonucleotide contains one or more dG nucleobases that complement the one or more methylated dC nucleobases). In embodiments, the probe oligonucleotide targets one or more CpG sites in the template single-stranded nucleic acid sequence. In embodiments, the probe oligonucleotide targets one CpG site in the template single-stranded nucleic acid sequence. In embodiments, the probe oligonucleotide targets methylated and/or non-methylated cytosines outside of a CpG site in the template single-stranded nucleic acid sequence. Databases and software for determining the location of CpG islands may be referenced when designing probe oligonucleotides targeting methylated or non-methylated cytosine nucleobases present in CpG sites, for example, the DataBase of CpG islands and Analytical Tool (DBCAT) (dbcat.cgm.ntu.edu.tw), see Kuo H C et al. J. Comput. Biol. 2011; 18(8): 1013-7), or CpGcluster, see Hackenberg M et al. BMC Bioinformatics. 2006; 7:446.

In embodiments, the probe oligonucleotide includes a sequence capable of hybridizing to an oncogene and/or tumor suppressor gene sequence, or a portion thereof. Non-limiting examples of oncogenes and tumor suppressor genes include the ABLI gene, AKT1 gene, ALK gene, APC gene, ATM gene, BRAF gene, BRCA gene, CDH1 gene, CDKN2A gene, CSFIR gene, CTNNB1 gene, EGFR gene, ERBB2 gene, ERBB4 gene, EZH2 gene, FBXW7 gene, FGFR1 gene, FGFR2 gene, FGFR3 gene, FLT3 gene, GNA11 gene, GNAQ gene, GNAS gene, HNFIA gene, HRAS gene, IDH1 gene, IDH2 gene, JAK2 gene, JAK3 gene, KDR gene, KIT gene, KRAS gene, MET gene, MLHI gene, MPL gene, NOTCHI gene, NPM1 gene, NRAS gene, PDGFRA gene, PIK3CA gene, PTEN genc, PTPN11 gene, RB1 gene, RET gene, SMAD4 gene, SMARCBI gene, SMO gene, SRC gene, STK11 gene, TP53 gene, VHL gene, or a portion thereof.

In embodiments, the first adapter is a Y-adapter. In embodiments, the second adapter is a Y-adapter. In embodiments, the Y-adapter includes (i) a first strand having a 5′-arm and a 3′-portion, and (ii) a second strand having a 5′-portion and a 3′-arm, wherein the 3′-portion of the first strand is substantially complementary to the 5′-portion of the second strand, and the 5′-arm of the first strand is not substantially complementary to the 3′-arm of the second strand. In embodiments, the 5′-arm of the first strand or the 3′-arm of the second strand of the Y-adapter includes a melting temperature (Tm) in a range of 60-85° C. In embodiments, the blocking primer anneals to the 5′-portion of the second strand of the Y-adapter.

In embodiments, the first adapter is a hairpin adapter. In some embodiments, the hairpin adapter includes a 5′-end, a 5′-portion, the loop, a 3′-portion and a 3′-end, and the 5′-portion of the hairpin adapter is substantially complementary to the 3′-portion of the hairpin adapter. In embodiments, the blocking primer anneals to a sequence within a loop of the first adapter.

In embodiments, the first adapter is a first hairpin adapter. In embodiments, the first hairpin adapter includes a 5′-end, a 5′-portion, a loop, a 3′-portion and a 3′-end, and the 5′-portion of the second hairpin adapter is substantially complementary to the 3′-portion of the second hairpin adapter. In embodiments, the blocking primer anneals to a sequence within the loop of the first hairpin adapter. In embodiments, the blocking primer anneals to a sequence within the first hairpin adapter.

In embodiments, the second adapter is a second hairpin adapter. In embodiments, the second hairpin adapter includes a 5′-end, a 5′-portion, a loop, a 3′-portion and a 3′-end, and the 5′-portion of the second hairpin adapter is substantially complementary to the 3′-portion of the second hairpin adapter. In embodiments, the blocking primer anncals to a sequence within the loop of the second hairpin adapter. In embodiments, the blocking primer anneals to a sequence within the second hairpin adapter.

In some embodiments, the first adapter is a Y-adapter, and annealing a blocking primer includes: (i) hybridizing a blocking primer to a single-stranded portion of the Y-adapter, and (ii) extending the blocking primer with a strand-displacing polymerase that terminates extension within a loop of the hairpin adapter at a terminating nucleotide.

In embodiments, the second adapter is a Y-adapter, and annealing a blocking primer includes: (i) hybridizing a blocking primer to a single-stranded portion of the Y-adapter, and (ii) extending the blocking primer with a strand-displacing polymerase that terminates extension within a loop of the first hairpin adapter at a terminating nucleotide.

In embodiments, the second adapter is a second hairpin adapter, and annealing a blocking primer includes: (i) hybridizing a blocking primer within a loop of the second hairpin adapter, and (ii) extending the blocking primer with a strand-displacing polymerase that terminates extension within a loop of the first hairpin adapter at a terminating nucleotide.

In embodiments, the terminating nucleotide includes a removable group that blocks progression of the strand-displacing polymerase, and further wherein the terminating nucleotide is treated to release the removable group prior to sequencing. Any of a variety of suitable modifications capable of terminating strand extensions may be used. In general, the terminating nucleotide is the nucleotide position that is modified to inhibit strand extension. The terminating nucleotide may or may not be a nucleotide analog. Thus, a terminating nucleotide is not necessarily chemically modified. For example, a terminating nucleotide may be a naturally occurring nucleotide, but is bound by another factor that inhibits strand extension (such as a sequence-specific binding protein). Any of a variety of suitable chemical modifications and blocking groups may be used. In embodiments, the terminating nucleotide is a nucleotide analog. In embodiments, the terminating nucleotide is an RNA nucleotide. Non-limiting examples include C3′-modifications, C2′-modifications, and phosphorodithioates.

In embodiments, the removable group is a polymer or a protein joined to the terminating nucleotide by a cleavable linker. In embodiments, the removable group is a polymer, such as a dendrimer. Non-limiting examples of polymers include PEG, polyethyleneimine, and poly(amidoamide). In embodiments, the protein is a bovine serum albumin (BSA) or a synthetic version of BSA.

In embodiments, the removable group is a protein that is non-covalently complexed to the terminating nucleotide, and further wherein releasing the protein includes a change in reaction conditions to disrupt the complex. The nature of the change in reaction conditions will depend on the nature of the protein complexed to the terminating nucleotide. In embodiments, the change in reaction conditions includes a change in temperature. In embodiments, the change in reaction conditions includes a change in buffer conditions, such as an increase in salt concentration. In embodiments, the change in reactions conditions includes the addition of another agent that competes with, inhibits, or degrades the protein.

In embodiments, the protein is a first member of a binding pair complexed with a second member of the binding pair that is linked to the terminating nucleotide. In embodiments, the protein is a single-stranded binding protein that recognizes a sequence within the loop of the hairpin adapter. In embodiments, the binding pair is a binding pair as described with respect to other aspects disclosed herein, including with respect to methods of sequencing described above.

In embodiments, the terminating nucleotide is a first nucleotide analog that base pairs with a second nucleotide analog, and the second nucleotide analog is not present in the primer extension reaction, such that primer extension terminates.

In some embodiments, each strand of a Y-adapter, each of the non-complementary arms of a Y-adapter, or a duplex portion of a Y-adapter has a length independently selected from at least 5, at least 10, at least 15, at least 25, and at least 40 nucleotides. In some embodiments, each strand of a Y-adapter, each of the non-complementary arms of a Y-adapter, or a duplex portion of a Y-adapter has a length in a range independently selected from 15 to 500 nucleotides, 15-250 nucleotides, 15 to 200 nucleotides, 15 to 150 nucleotides, 20 to 100 nucleotides, 20 to 50 nucleotides and 10-50 nucleotides. In embodiments, one or both non-complementary arms of the Y-adapter is about or at least about 10, 15, 20, 25, 30, 35, 40, 45, 50, or more nucleotides in length. In embodiments, one or both non-complementary arms of the Y-adapter is about or at least about 20 nucleotides in length. In embodiments, one or both non-complementary arms of the Y-adapter is about or at least about 30 nucleotides in length. In embodiments, one or both non-complementary arms of the Y-adapter is about or at least about 40 nucleotides in length. In embodiments, the duplex portion of a Y-adapter is about or at least about 5, 10, 15, 20, 25, 30, or more nucleotides in length. In embodiments, the duplex portion of a Y-adapter is about 5-50, 5-25, or 10-15 nucleotides in length. In embodiments, the duplex portion of a Y-adapter is about or at least about 10 nucleotides in length. In embodiments, the duplex portion of a Y-adapter is about or at least about 15 nucleotides in length. In embodiments, the duplex portion of a Y-adapter is about or at least about 12 nucleotides in length. In embodiments, the duplex portion of a Y-adapter is about or at least about 20 nucleotides in length.

In some embodiments, a Y-adapter includes a first end including a duplex region including double stranded nucleic acid, and a second end including a forked region, where the first end is configured for ligation to an end of double stranded nucleic acid (e.g., a nucleic acid fragment, e.g., a library insert). In embodiments, a duplex end of a Y-adapter includes a 5′-overhang or a 3′-overhang that is complementary to a 3′-overhang or a 5′-overhang of an end of a double stranded nucleic acid. In some embodiments, a duplex end of a Y-adapter includes a blunt end that can be ligated to a blunt end of a double stranded nucleic acid. In certain embodiment, a duplex end of a Y-adapter includes a 5′-end that is phosphorylated.

In some embodiments, the first and/or second adapter (e.g., one or both strands of a Y-adapter) include one or more of a primer binding site, a capture nucleic acid binding site (e.g., a nucleic acid sequence complementary to a capture nucleic acid), a UMI, a sample barcode, a sequencing adapter, a label, a binding motif, the like or combinations thereof. In some embodiments, a non-complementary portion (e.g., 5′-arm and/or 3′-arm) of a Y-adapter includes one or more of a primer binding site, a capture nucleic acid binding site (e.g., a nucleic acid sequence complementary to a capture nucleic acid), a UMI, a sample barcode, a sequencing adapter, a label, a binding motif, the like or combinations thereof. In certain embodiments, a non-complementary portion of a Y-adapter includes a primer binding site. In certain embodiments, a non-complementary portion of a Y-adapter includes a binding site for a capture nucleic acid. In certain embodiments, a non-complementary portion of a Y-adapter includes a primer binding site and a UMI. In certain embodiments, a non-complementary portion of a Y-adapter includes a binding motif. In embodiments, the first and/or second adapter (e.g., one or both strands of a Y-adapter) does not include a UMI or sample barcode.

In certain embodiments, a complementary strand (e.g., a 3′-portion or 5′-portion) of a Y-adapter includes a primer binding site. In certain embodiments, a complementary strand (e.g., a 3′-portion or 5′-portion) of a Y-adapter includes a binding site for a capture nucleic acid. In certain embodiments, a complementary strand (e.g., a 3′-portion or 5′-portion) of a Y-adapter includes a primer binding site and a UMI. In certain embodiments, a complementary strand (e.g., a 3′-portion or 5′-portion) of a Y-adapter includes a binding motif.

In some embodiments, each of the non-complementary portions (i.e., arms) of a Y-adapter independently have a predicted, calculated, mean, average or absolute melting temperature (Tm) that is greater than 50° C., greater than 55° C., greater than 60° C., greater than 65° C., greater than 70° C. or greater than 75° C. In some embodiments, each of the non-complementary portions of a Y-adapter independently have a predicted, estimated, calculated, mean, average or absolute melting temperature (Tm) that is in a range of 50-100° C., 55-100° C., 60-100° C., 65-100° C., 70-100° C., 55-95° C., 65-95° C., 70-95° C., 55-90° C., 65-90° C., 70-90° C., or 60-85° C. In embodiments, the Tm is about or at least about 70° C. In embodiments, the Tm is about or at least about 75° C. In embodiments, the Tm is about or at least about 80° C. In embodiments, the Tm is a calculated Tm. Tm's are routinely calculated by those skilled in the art, such as by commercial providers of custom oligonucleotides. In embodiments, the Tm for a given sequence is determined based on that sequence as an independent oligo. In embodiments, Tm is calculated using web-based algorithms, such as Primer3 and Primer3Plus (www.bioinformatics.nl/cgi-bin/primer3plus/primer3plus.cgi) using default parameters. The Tm of a non-complementary portion of a Y-adapter can be changed (e.g., increased) to a desired Tm using a suitable method, for example by changing (e.g., increasing) GC content, changing (e.g., increasing) length and/or by the inclusion of modified nucleotides, nucleotide analogs and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, in some embodiments, each of the non-complementary portion of a Y-adapter independently include one or more modified nucleotides, nucleotide analogs and/or modified nucleotides bonds.

In some embodiments, each of the non-complementary portions of a Y-adapter independently include a GC content of greater than 40%, greater than 50%, greater than 55%, greater than 60% greater than 65% or greater than 70%. In certain embodiments, each of the non-complementary portions of a Y-adapter independently include a GC content in a range of 40-100%, 50-100%, 60-100% or 70-100%. In embodiments, one or both non-complementary portions of a Y-adapter have a GC content of about or more than about 40%. In embodiments, one or both non-complementary portions of a Y-adapter have a GC content of about or more than about 50%. In embodiments, one or both non-complementary portions of a Y-adapter have a GC content of about or more than about 60%. Non-base modifiers can also be incorporated into a non-complementary portion of a Y-adapter to increase Tm, non-limiting examples of which include a minor grove binder (MGB), spermine, G-clamp, a Uaq anthraquinone cap, the like or combinations thereof.

In certain embodiments, a duplex region of a Y-adapter includes a predicted, estimated, calculated, mean, average or absolute Tm in a range of 30-70° C., 35-65° C., 35-60° C., 40-65° C., 40-60° C., 35-55° C., 40-55° C., 45-50° C. or 40-50° C. In embodiments, the Tm of a duplex region of the Y-adapter is about or more than about 30° C. In embodiments, the Tm of a duplex region of the Y-adapter is about or more than about 35° C. In embodiments, the Tm of a duplex region of the Y-adapter is about or more than about 40° C. In embodiments, the Tm of a duplex region of the Y-adapter is about or more than about 45° C. In embodiments, the Tm of a duplex region of the Y-adapter is about or more than about 50° C.

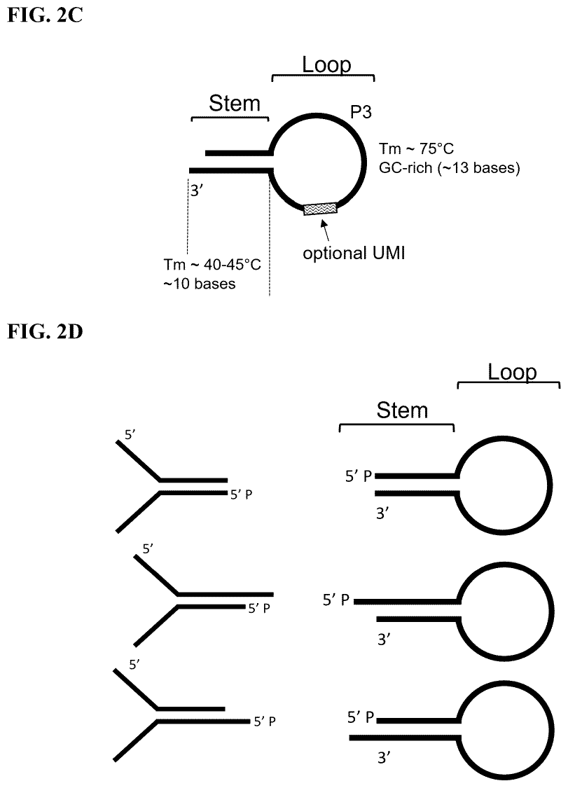

In some embodiments, an adapter is hairpin adapter. In some embodiments, a hairpin adapter includes a single nucleic acid strand including a stem-loop structure. A hairpin adapter can be any suitable length. In some embodiments, a hairpin adapter is at least 40, at least 50, or at least 100 nucleotides in length. In some embodiments, a hairpin adapter has a length in a range of 45 to 500 nucleotides, 75-500 nucleotides, 45 to 250 nucleotides, 60 to 250 nucleotides or 45 to 150 nucleotides. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, a hairpin adapter includes a structure described herein (e.g., FIGS. 2B-2D). In some embodiments, the second adapter includes a sample barcode sequence, a molecular identifier sequence, or both a sample barcode sequence and a molecular identifier sequence. In some embodiments, the second adapter includes a sample barcode sequence.

In some embodiments, a duplex region or stem portion of a hairpin adapter includes an end that is configured for ligation to an end of double stranded nucleic acid (e.g., a nucleic acid fragment, e.g., a library insert). In embodiments, an end of a duplex region or stem portion of a hairpin adapter includes a 5′-overhang or a 3′-overhang that is complementary to a 3′-overhang or a 5′-overhang of one end of a double stranded nucleic acid. In some embodiments, an end of a duplex region or stem portion of a hairpin adapter includes a blunt end that can be ligated to a blunt end of a double stranded nucleic acid. In certain embodiment, an end of a duplex region or stem portion of a hairpin adapter includes a 5′-end that is phosphorylated. In some embodiments, a stem portion of a hairpin adapter is at least 15, at least 25, or at least 40 nucleotides in length. In some embodiments, a stem portion of a hairpin adapter has a length in a range of 15 to 500 nucleotides, 15-250 nucleotides, 15 to 200 nucleotides, 15 to 150 nucleotides, 20 to 100 nucleotides or 20 to 50 nucleotides.

In embodiments, ligating includes ligating both the 3′ end and the 5′ end of the duplex region of the second adapter to the double stranded nucleic acid. In embodiments, ligating includes ligating either the 3′ end or the 5′ end of the duplex region of the second adapter to the double stranded nucleic acid. In embodiments, ligating includes ligating the 5′ end of the duplex region of the second adapter to the double stranded nucleic acid and not the 3′ end of the duplex region.

In some embodiments, a loop of a hairpin adapter includes one or more of a primer binding site, a capture nucleic acid binding site (e.g., a nucleic acid sequence complementary to a capture nucleic acid), a UMI, a sample barcode, a sequencing adapter, a label, the like or combinations thereof. In certain embodiments, a loop of a hairpin adapter includes a primer binding site. In certain embodiments, a loop of a hairpin adapter includes a primer binding site and a UMI. In certain embodiments, a loop of a hairpin adapter includes a binding motif.

In some embodiments, a loop of a hairpin adapter has a predicted, calculated, mean, average or absolute melting temperature (Tm) that is greater than 50° C., greater than 55° C., greater than 60° C., greater than 65° C., greater than 70° C. or greater than 75° C. In some embodiments, a loop of a hairpin adapter has a predicted, estimated, calculated, mean, average or absolute melting temperature (Tm) that is in a range of 50-100° C., 55-100° C., 60-100° C., 65-100° C., 70-100° C., 55-95° C., 65-95° C., 70-95° C., 55-90° C., 65-90° C., 70-90° C., or 60-85° C. In embodiments, the Tm of the loop is about 65° C. In embodiments, the Tm of the loop is about 75° C. In embodiments, the Tm of the loop is about 85° C. The Tm of a loop of a hairpin adapter can be changed (e.g., increased) to a desired Tm using a suitable method, for example by changing (e.g., increasing GC content), changing (e.g., increasing) length and/or by the inclusion of modified nucleotides, nucleotide analogs and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, in some embodiments, a loop of a hairpin adapter includes one or more modified nucleotides, nucleotide analogs and/or modified nucleotides bonds.

In some embodiments, a loop of a hairpin adapter independently includes a GC content of greater than 40%, greater than 50%, greater than 55%, greater than 60% greater than 65% or greater than 70%. In certain embodiments, a loop of a hairpin adapter independently includes a GC content in a range of 40-100%, 50-100%, 60-100% or 70-100%. In embodiments, the loops has a GC content of about or more than about 40%. In embodiments, the loops has a GC content of about or more than about 50%. In embodiments, the loops has a GC content of about or more than about 60%. Non-base modifiers can also be incorporated into a loop of a hairpin adapter to increase Tm, non-limiting examples of which include a minor grove binder (MGB), spermine, G-clamp, a Uaq anthraquinone cap, the like or combinations thereof. A loop of a hairpin adapter can be any suitable length. In some embodiments, a loop of a hairpin adapter is at least 15, at least 25, or at least 40 nucleotides in length. In some embodiments, a hairpin adapter has a length in a range of 15 to 500 nucleotides, 15-250 nucleotides, 20 to 200 nucleotides, 30 to 150 nucleotides or 50 to 100 nucleotides.

In certain embodiments, a duplex region or stem region of a hairpin adapter includes a predicted, estimated, calculated, mean, average or absolute Tm in a range of 30-70° C., 35-65° C., 35-60° C., 40-65° C., 40-60° C., 35-55° C., 40-55° C., 45-50° C. or 40-50° C. In embodiments, the Tm of the stem region is about or more than about 35° C. In embodiments, the Tm of the stem region is about or more than about 40° C. In embodiments, the Tm of the stem region is about or more than about 45° C. In embodiments, the Tm of the stem region is about or more than about 50° C.

In some embodiments, the first adapter is a hairpin adapter, and annealing a blocking primer includes: (i) hybridizing a blocking primer within a loop of the first hairpin adapter, and (ii) extending the blocking primer with a strand-displacing polymerase that terminates extension within a loop of the second hairpin adapter at a terminating nucleotide.

In embodiments, the Y-adapter portion of a Y-adapter-ligated double-stranded nucleic acid is formed from cleavage in the loop of a hairpin adapter (e.g., one or more adapters as described in U.S. Pat. No. 8,883,990, which is incorporated herein by reference for all purposes). For example, in embodiments disclosed herein relating to ligation to a Y-adapter, ligation may instead be to a hairpin adapter, followed by cleavage within the loop of the hairpin adapter to release two unpaired ends. In embodiments, a hairpin adapter includes one or more uracil nucleotide(s) in the loop, and cleavage in the loop may be accomplished by the combined activities of Uracil DNA glycosylase (UDG) and the DNA glycosylase-lyase Endonuclease VIII, or suitable cleavage conditions known in the art. UDG cleaves the glycosidic bond between the deoxyribose of the DNA sugar-phosphate backbone and the uracil base, and Endonuclease VIII cleaves the AP site, effectively cleaving the loop. In embodiments, the hairpin adapter includes a recognition sequence for a compatible restriction enzyme. In embodiments, the hairpin adapter includes one or more ribonucleotides and cleavage in the loop is accomplished by RNase H. In embodiments, the loop of the hairpin adapter includes a cleavable linkage (e.g., a cleavable site) that is positioned between two non-complementary regions of the loop. In embodiments, the non-complementary region that is 5′ of the cleavable linkage includes a primer binding site that is in the range of 8 to 100 nucleotides in length. In embodiments, the first adapter is a hairpin adapter, wherein the hairpin adapter includes a cleavable site in the loop. In embodiments, the first adapter is a first hairpin adapter and the second adapter is a hairpin adapter, wherein only the first hairpin adapter includes a cleavable site in the loop.

In some embodiments, a method includes sequencing a template described herein. In some embodiments, the sequencing includes contacting the template with a suitable polymerase. In certain embodiments, the polymerase is in an aqueous phase. In certain embodiments, the polymerase is soluble in an aqueous solution. In some embodiments, the polymerase is not attached to a substrate. In some embodiments, the polymerase is attached to a substrate. In embodiments, the polymerase is a mutant polymerase capable of incorporating modified nucleotides.

In embodiments, the terminating nucleotide includes a removable group that blocks progression of the strand-displacing polymerase, and further wherein the terminating nucleotide is treated to release the removable group prior to sequencing.

In embodiments, the terminating nucleotide is an RNA nucleotide.

In embodiments, annealing a blocking primer includes (i) forming a complex including a portion of the double-stranded nucleic acid, a blocking primer, and a homologous recombination complex including a recombinase, (ii) releasing the recombinase, and (iii) extending the blocking primer with a strand-displacing polymerase.

In embodiments, annealing the blocking primer includes (i) forming a complex with the double-stranded nucleic acid, the blocking primer, and a homologous recombination complex including a recombinase, (ii) releasing the recombinase, and (iii) extending the blocking primer with a strand-displacing polymerase.

In embodiments, (i) annealing a blocking primer includes forming a complex including a portion of the double-stranded nucleic acid, a probe oligonucleotide, and a homologous recombination complex including a recombinase, and (ii) annealing a probe oligonucleotide to the second template single-stranded nucleic acid includes releasing the recombinase.

In embodiments, the homologous recombination complex further includes a loading factor, a single-stranded binding (SSB) protein, or both.

In embodiments, the probe oligonucleotide is covalently attached to a substrate (e.g., a particle).

In embodiments, the probe oligonucleotide is labeled with a first member of a binding pair, and separating the probe-hybridized double-stranded nucleic acid from nucleic acids not hybridized to a probe includes capturing the probe with a second member of the binding pair. In embodiments, (i) the first member of the binding pair is biotin and the second member of the binding pair is avidin or streptavidin, or (ii) the second member of the binding pair is biotin and the first member of the binding pair is avidin or streptavidin.

In embodiments, the double-stranded nucleic acid is a cell-free DNA (cfDNA) or circulating tumor DNA (ctDNA). In embodiments, the double-stranded nucleic acid is a cell-free DNA (cfDNA). In embodiments, the double-stranded nucleic acid is a circulating tumor DNA (ctDNA). In embodiments, the double-stranded nucleic acid is from a FFPE sample. In embodiments, the double-stranded nucleic acid is extracted from plasma or from peripheral blood mononuclear cells (PBMCs). In embodiments, the double-stranded nucleic acid is 50 to 100 bp in length. In embodiments, the double-stranded nucleic acid includes genomic DNA, complementary DNA (cDNA), cell-free DNA (cfDNA), messenger RNA (mRNA), transfer RNA (tRNA), ribosomal RNA (rRNA), cell-free RNA (cfRNA), or noncoding RNA (ncRNA).

In certain embodiments, a method includes annealing a first primer to a 3′-portion of a template described herein, or to a 3′-end of a complementary sequence of a template described herein (e.g., a 3′ end of an amplicon of a template). In certain embodiments, a method includes annealing a first primer to a 3′-portion of a template described herein, where the 3′-portion of the template includes a portion of an adapter (e.g., a first adapter). In certain embodiments, a method includes annealing a first primer to a 3′-portion of a template described herein, where the 3′-portion of the template includes a portion of a Y-adapter. In certain embodiments, a method includes annealing a first primer to a 3′-arm of a Y-adapter of a template described herein, where the 3′-arm of the adapter includes a primer binding site for the first primer. In certain embodiments, a method includes annealing a first primer to a 5′-portion of a second strand of a Y-adapter of a template described herein, where the 5′-portion of the adapter includes a primer binding site for the first primer.

In embodiments where a template includes two hairpin adapters located on opposing sides of a double stranded nucleic acid, a method includes annealing a first primer to a portion of a first adapter of a template described herein. In certain embodiments, a method includes annealing a first primer to a loop of a first hairpin adapter of a template described herein, where the loop of the adapter includes a first primer binding site for the first primer. In some embodiments, a method includes annealing a first primer to a stem of a first hairpin adapter of a template described herein, where the stem of the adapter includes a first primer binding site for the first primer.

In certain embodiments, a method includes sequencing a first portion of a nucleic acid template by extending a first primer, thereby generating a first read including a first nucleic acid sequence of at least a first portion of the double stranded nucleic acid. In some embodiments, a method includes sequencing a reverse strand of a nucleic acid template by extending a first primer, thereby generating a first read including a nucleic acid sequence of at least a portion of the reverse strand of a double stranded nucleic acid.

In certain embodiments, a method includes sequencing a second portion of a nucleic acid template by extending a second primer, thereby generating a second read including a second nucleic acid sequence of at least a second portion of the double stranded nucleic acid. In some embodiments, a method includes sequencing a forward strand of a nucleic acid template by extending a second primer, thereby generating a second read including a nucleic acid sequence of at least a portion of the forward strand of a double stranded nucleic acid. In some embodiments, a method includes annealing a second primer to the nucleic acid template, wherein the second primer includes a sequence that is complementary to a primer binding sequence located within a loop of the hairpin adapter (i.e., second adapter). In certain embodiments, a second primer is annealed to a loop of the hairpin adapter (i.e., second adapter) and a second portion of the nucleic acid template (e.g., the forward strand) is sequenced by extending the second primer, thereby generating a second read of the nucleic acid template.

In some embodiments, a method includes (i) hybridizing a first primer to a 3′-portion of a template where the 3′ portion of the template includes a portion of a Y-adapter, (ii) sequencing a portion of a first strand of a double-stranded nucleic acid, (iii) hybridizing a second primer to a loop or stem of a hairpin adapter of the template, and (iv) sequencing a portion of a second strand of the double-stranded nucleic acid. In some embodiments, the methods herein can be applied to an amplicon or copy of a template (or complement thereof), as well as to the original template.

In some embodiments, the step of sequencing a first portion of a nucleic acid template as described herein is conducted before, after and/or during the step of sequencing a second portion of a nucleic acid template as described herein. For example, in certain embodiments, a second primer is annealed to a loop or stem region of a hairpin adapter and a first portion of a double stranded nucleic acid insert is sequenced by extending the second primer, followed by annealing a first primer to a 3′-end of the template including a portion of a Y-adapter, and sequencing a second portion of the double stranded nucleic acid insert by extending the first primer.

In certain embodiments, a method includes generating amplicons of the nucleic acid template (e.g., the nucleic acid ligated to a first and second adapter, as described herein). Amplicons may be generated using a suitable amplification method. In certain embodiments, amplicons of a template are generated using a polymerase chain reaction or a rolling circle amplification method, or a combination thereof. In certain embodiments, amplicons are generated using a polymerase chain reaction. In certain embodiments, amplicons are generated using a bridge PCR amplification method. In embodiments, amplicons are generated using thermal bridge polymerase chain reaction (t-bPCR) amplification. In embodiments, amplicons are generated using a chemical bridge polymerase chain reaction (c-bPCR) amplification. Chemical bridge polymerase chain reactions include fluidically cycling a denaturant (e.g., formamide) and maintaining the temperature within a narrow temperature range (e.g., +/−5° C.). In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions. In embodiments, generating amplicons includes a thermal bridge polymerase chain reaction (t-bPCR) amplification. In embodiments, the plurality of cycles includes thermally cycling between (i) about 85° C. for about 15-30 sec for denaturation, and (ii) about 65° C. for about 1 minute for annealing/extension of the primer. In embodiments, the plurality of cycles includes thermally cycling between (i) about 85° C. for about 15-30 sec for denaturation, and (ii) about 65° C. for about 30 seconds for annealing/extension of the primer.

Provided herein in an aspect is a method of amplifying a double-stranded nucleic acid template. In embodiments, the method includes (a) ligating a first adapter to a first end of the double stranded nucleic acid, and ligating a second adapter to a second end of the double stranded nucleic acid, wherein the second adapter is a hairpin adapter, thereby forming a nucleic acid template; (b) annealing a first primer to the nucleic acid template, wherein the first primer includes a sequence that is complementary to a portion of the first adapter, or a complement thereof, and is not substantially complementary to a portion of the second adapter; (c) generating amplicons using a suitable amplification method. In embodiments, the method provides a copy of the nucleic acid template as a single-stranded molecule of DNA, and, advantageously, contains both forward and reverse strands of the original double-stranded DNA molecule. In embodiments, the method further includes sequencing the amplicons using a method known in the art or described herein.

In embodiments, the method includes amplifying a double stranded nucleic acid including a first strand and a second strand, the method including: (a) ligating a first adapter to a first end of the double stranded nucleic acid wherein the first adapter is a Y adapter including (i) a first strand having a 5′-arm and a 3′-portion, and (ii) a second strand having a 5′-portion and a 3′-arm, wherein the 3′-portion of the first strand is substantially complementary to the 5′-portion of the second strand, and the 5′-arm of the first strand is not substantially complementary to the 3′-arm of the second strand, and ligating a second adapter to a second end of the double stranded nucleic acid, wherein the second adapter is a hairpin adapter, thereby forming a nucleic acid template; (b) annealing a primer to the nucleic acid template, wherein the first primer includes a sequence that is complementary to a portion of the first adapter, or a complement thereof, and is not substantially complementary to a portion of the second adapter, or a complement thereof; and (c) amplifying the nucleic acid template by extending the primer using a strand-displacing polymerase, thereby generating an amplicon (e.g., a single-stranded amplicon) including a complement of the first and second strand of the double stranded nucleic acid. In embodiments, the amplicon is a contiguous strand of DNA that contains the first and second strand of the double-stranded nucleic acid. In embodiments, the amplicon is a continuous strand lacking free 5′ and 3′ ends. In embodiments, the amplicon is a single-stranded amplicon. In embodiments, after step (a) the method includes amplifying the nucleic acid template to generate a plurality of nucleic acid templates using a polymerase chain reaction.

In embodiments, amplifying the nucleic acid template is on a solid support including a plurality of primers attached to the solid support, wherein the plurality of primers include a plurality of forward primers with complementarity to a complement of the first strand of the Y adapter (e.g., the 5′ arm portion) and a plurality of reverse primers with complementarity to the second strand of the Y adapter (e.g., the 3′ arm portion), and the amplifying includes a plurality of cycles of strand denaturation, primer hybridization, and primer extension, thereby generating a plurality of forward amplicons and a plurality of reverse amplicons.

In embodiments, the method includes amplifying the nucleic acid template with bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR, or combinations of the methods. In embodiments, generating an amplification product includes bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR on particles, or combinations of the methods. In embodiments, generating an amplification product includes a bridge polymerase chain reaction amplification. In embodiments, generating an amplification product includes a thermal bridge polymerase chain reaction (t-bPCR) amplification. In embodiments, generating an amplification product includes a chemical bridge polymerase chain reaction (c-bPCR) amplification. Chemical bridge polymerase chain reactions include fluidically cycling a denaturant (e.g., formamide) and maintaining the temperature within a narrow temperature range (e.g., +/−5° C.). In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions.

In embodiments, the plurality of forward primers are covalently attached to the solid support via a first linker and the reverse primers are covalently attached to the solid support via a second linker. The linker tethering the polynucleotide strands may be any linker capable of localizing nucleic acids to arrays. The linkers may be the same, or the linkers may be different. Solid-supported molecular arrays have been generated previously in a variety of ways, for example, the attachment of biomolecules (e.g., proteins and nucleic acids) to a variety of substrates (e.g., glass, plastics, or metals) underpins modern microarray and biosensor technologies employed for genotyping, gene expression analysis and biological detection. Silica-based substrates are often employed as supports on which molecular arrays are constructed, and functionalized silanes are commonly used to modify glass to permit a click-chemistry enabled linker to tether the biomolecule.

In embodiments, the method further includes removing the plurality of reverse amplicons, annealing a primer to the amplicon (e.g., the first amplicon), wherein the first primer includes a sequence that is complementary to a portion of the amplicon, or a complement thereof, and sequencing a portion of the first amplicon by extending the primer, thereby generating a sequencing read including a first nucleic acid sequence of at least a first portion of the double stranded nucleic acid. In embodiments, the method further includes removing the plurality of forward amplicons, annealing a primer to the amplicon (e.g., the first amplicon), wherein the first primer includes a sequence that is complementary to a portion of the first amplicon, or a complement thereof, and sequencing a portion of the first amplicon by extending the primer, thereby generating a sequencing read including a first nucleic acid sequence of at least a first portion of the double stranded nucleic acid.

In embodiments, amplifying includes incubation in a denaturant. In embodiments, the denaturant is acetic acid, ethylene glycol, hydrochloric acid, nitric acid, formamide, guanidine, sodium salicylate, sodium hydroxide, dimethyl sulfoxide (DMSO), propylene glycol, urea, or a mixture thereof. In embodiments, the denaturant is an additive that lowers a DNA denaturation temperature. In embodiments, the denaturant is betaine, dimethyl sulfoxide (DMSO), ethylene glycol, formamide, glycerol, guanidine thiocyanate, 4-methylmorpholine 4-oxide (NMO), or a mixture thereof. In embodiments, the denaturant is betaine, dimethyl sulfoxide (DMSO), ethylene glycol, formamide, glycerol, guanidine thiocyanate, or 4-methylmorpholine 4-oxide (NMO).

In embodiments, amplifying includes a plurality of cycles of strand denaturation, primer hybridization, and primer extension. Although each cycle will include each of these three events (denaturation, hybridization, and extension), events within a cycle may or may not be discrete. For example, each step may have different reagents and/or reaction conditions (e.g., temperatures). Alternatively, some steps may proceed without a change in reaction conditions. For example, extension may proceed under the same conditions (e.g., same temperature) as hybridization. After extension, the conditions are changed to start a new cycle with a new denaturation step, thereby amplifying the amplicons. Primer extension products from an earlier cycle may serve as templates for a later amplification cycle. In embodiments, the plurality of cycles is about 5 to about 50 cycles. In embodiments, the plurality of cycles is about 10 to about 45 cycles. In embodiments, the plurality of cycles is about 10 to about 20 cycles. In embodiments, the plurality of cycles is about 20 to about 30 cycles. In embodiments, the plurality of cycles is 10 to 45 cycles. In embodiments, the plurality of cycles is 10 to 20 cycles. In embodiments, the plurality of cycles is 20 to 30 cycles. In embodiments, the plurality of cycles is about 10 to about 45 cycles. In embodiments, the plurality of cycles is about 20 to about 30 cycles.

In some embodiments, an amplification method includes attaching a nucleic acid template described herein to a substrate. In certain embodiments, attaching a nucleic acid template to a substrate includes annealing a capture nucleic acid to a template. In some embodiments, a capture nucleic acid anneals to a complementary sequence that is present on an adapter portion of a template (e.g., a Y-adapter or hairpin adapter). In certain embodiments, a capture nucleic acid anneals to a primer binding site located on a Y-adapter portion of a template described herein. A capture nucleic acid may anneal to a portion of a Y-adapter on or near the 3′-end or 3′-side of a template. In some embodiments, a capture nucleic acid anneals to a 3′-arm of a Y-adapter on a template.

In embodiments, the nucleic acid template is provided in a clustered array. In embodiments, the clustered array includes a plurality of amplicons localized to discrete sites on a solid support. In embodiments, the solid support is a bead. In embodiments, the solid support is substantially planar. In embodiments, the solid support is contained within a flow cell. Flow cells provide a convenient format for housing an array of clusters produced by the methods described herein, in particular when subjected to an SBS or other detection technique that involves repeated delivery of reagents in cycles. For example, to initiate a first SBS cycle, one or more labeled nucleotides and a DNA polymerase in a buffer, can be flowed into/through a flow cell that houses an array of clusters. The clusters of an array where primer extension causes a labeled nucleotide to be incorporated can then be detected. Optionally, the nucleotides can further include a reversible termination moiety that temporarily halts further primer extension once a nucleotide has been added to a primer. For example, a nucleotide analog having a reversible terminator moiety can be added to a primer such that subsequent extension cannot occur until a deblocking agent (e.g., a reducing agent) is delivered to remove the moiety. Thus, for embodiments that use reversible termination, a deblocking reagent (e.g., a reducing agent) can be delivered to the flow cell (before, during, or after detection occurs). Washes can be carried out between the various delivery steps as needed. The cycle can then be repeated N times to extend the primer by N nucleotides, thereby detecting a sequence of length N. Example SBS procedures, fluidic systems and detection platforms that can be readily adapted for use with an array produced by methods of the present disclosure are described, for example, in Bentley et al., Nature 456:53-59 (2008), US Patent Publication 2018/0274024, WO 2017/205336, US Patent Publication 2018/0258472, each of which are incorporated herein in their entirety for all purposes

In some embodiments, an amplification method includes annealing a primer or capture nucleic acid to a portion of a Y-adapter on or near a 3′-end of a template, and extending the primer using a polymerase, thereby generating a first amplicon (first copy) of the template. In certain embodiments, a 3′-end of the first amplicon is annealed to another primer or capture nucleic acid, which is then extended to generate a second amplicon. The amplification process continues until a plurality of first amplicons (e.g., a set of first amplicons) and a plurality of second amplicons (e.g., a set of second amplicons) are generated. In embodiments, a bridge PCR amplification method produces a first set of amplicons that are complementary to an original template, and a second set of amplicons that have nucleic acid sequences substantially identical to the original template, where both the first and second sets of amplicons are attached to a substrate (e.g., a substrate of a flow cell). After bridge amplification, in certain embodiments, the first set of amplicons, or alternatively the second set of amplicons, are removed from a surface or substrate using a suitable method, usually by restriction enzyme. Cleaving one strand may be referred to as linearization. Suitable methods for linearization are known, and described in more detail in U.S. Patent Publication No. 2009/0118128, which is incorporated herein by reference in its entirety. For example, the first strand may be cleaved by exposing the first strand to a mixture containing a glycosylase and one or more suitable endonucleases. In embodiments, cleaving includes chemically cleaving one strand at a cleavable site. In embodiments, the cleavable site includes a diol linker, disulfide linker, photocleavable linker, abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent.

Any suitable enzymatic, chemical, or photochemical cleavage reaction may be used to cleave the cleavage site. The cleavage reaction may result in removal of a part or the whole of the strand being cleaved. Suitable cleavage means include, for example, restriction enzyme digestion, in which case the cleavage site is an appropriate restriction site for the enzyme which directs cleavage of one or both strands of a duplex template; RNase digestion or chemical cleavage of a bond between a deoxyribonucleotide and a ribonucleotide, in which case the cleavage site may include one or more ribonucleotides; chemical reduction of a disulfide linkage with a reducing agent (e.g., THPP or TCEP), in which case the cleavage site should include an appropriate disulfide linkage; chemical cleavage of a diol linkage with periodate, in which case the cleavage site should include a diol linkage; generation of an abasic site and subsequent hydrolysis, etc. In embodiments, the cleavage site is included in the surface immobilized primer (e.g., within the polynucleotide sequence of the primer). In embodiments, one strand of the double-stranded amplification product (or the surface immobilized primer) may include a diol linkage which permits cleavage by treatment with periodate (e.g., sodium periodate). It will be appreciated that more than one diol can be included at the cleavage site. One or more diol units may be incorporated into a polynucleotide using standard methods for automated chemical DNA synthesis. Polynucleotide primers including one or more diol linkers can be conveniently prepared by chemical synthesis. The diol linker is cleaved by treatment with any substance which promotes cleavage of the diol (e.g., a diol-cleaving agent). In embodiments, the diol-cleaving agent is periodate, e.g., aqueous sodium periodate (NaIO₄). Following treatment with the diol-cleaving agent (e.g., periodate) to cleave the diol, the cleaved product may be treated with a “capping agent” in order to neutralize reactive species generated in the cleavage reaction. Suitable capping agents for this purpose include amines, e.g., ethanolamine or propanolamine.

In embodiments, the method includes removing immobilized primers that do not contain a first or second strand of the nucleic acid template (i.e., unused primers) on a solid support. Methods of removing immobilized primers can include digestion using an enzyme with exonuclease activity. Removing unused primers may serve to increase the free volume and allow for greater accessibility. Removal of unused primers may also prevent opportunities for the newly released first strand to rehybridize to an available surface primer, producing a priming site off the available surface primer, thereby facilitating the “reblocking” of the released first strand.

In embodiments, generating the blocking strand includes a plurality of blocking primer extension cycles. In embodiments, generating the blocking strand includes extending the blocking primer by incorporating one or more nucleotides (e.g., dNTPs) using Bst large fragment (Bst LF) polymerase, Bst2.0 polymerase, Bsu polymerase, SD polymerase, Vent exo-polymerase, Phi29 polymerase, or a mutant thereof.

In embodiments, the method further includes removing the blocking strand and sequencing the first strand. In embodiments, the method further includes removing the blocking strand and sequencing the second strand. In embodiments, the method further includes removing both the blocking strand and the second strand prior to sequencing the first strand. In embodiments, the method further includes removing both the blocking strand and the first strand prior to sequencing the second strand. In embodiments, the method further includes removing one or more sequencing reads prior to sequencing the first strand or sequencing the second strand.

In embodiments, removing the blocking strand includes digesting the invasion strand using an exonuclease enzyme. In embodiments, removing the sequencing read includes digesting the sequencing read using an exonuclease enzyme. In embodiments, removing the first strand of the nucleic acid template includes cleaving a cleavable site in the at least first strand of the nucleic acid template. In embodiments, removing the first invasion strand and removing the second invasion strand includes enzymatically cleaving the second primer binding sequence at the cleavable site. In embodiments, the cleavable site includes a sequence that is specifically recognized by a restriction endonuclease. In embodiments, the exonuclease enzyme is a 3′-5′ exonuclease. In embodiments, the exonuclease enzyme is a 5′-3′ exonuclease. In embodiments, the 3′-5′ exonuclease is exonuclease I, exonuclease T, a proofreading polymerase, or a mutant thereof. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” In embodiments, the proofreading polymerase is a phi29 polymerase, or mutant thereof. In embodiments, the 5′-3′ exonuclease is lambda exonuclease, or a mutant thereof.

In embodiments, removing the blocking strand, removing the sequencing read, or removing both the blocking strand and the sequencing read includes incubation in a denaturant as described herein, for example, wherein the denaturant is a buffered solution including about 0% to about 50% dimethyl sulfoxide (DMSO); about 0% to about 50% ethylene glycol; about 0% to about 20% formamide; or about 0 to about 3M betaine, or a mixture thereof.

After removal of one of the sets of amplicons from the substrate, the other remaining set of substrate-attached amplicons is subjected to sequencing by annealing a first sequencing primer at the 3′-end (3′-region) of each of the amplicons (formerly a portion of the Y-adapter), and extending the first primer to obtain a sequence read of a 3′ portion of each of the amplicons, which includes a sequence of a first strand of the original double stranded insert. Before, during or after obtaining a sequence read of the 3′-portion of the amplicons, a second primer is annealed to the loop of each of the set of amplicons (i.e., the loop portion of the hairpin adapter used to make the template) and the second primer is used to obtain a second sequence read of a second portion of the amplicon, which includes a sequence of the opposite strand of the original doubled stranded insert. The process described above obtains a sequence read of both strands of the original double stranded nucleic acid insert from a single set of substantially identical amplicons. In some embodiments, sequencing method is complete at this stage and does not require another amplification step. Traditional methods of paired-end sequencing that utilize bridge amplification require a first amplification to obtain a first read of one strand of an insert followed by a second amplification to obtain a second read of the other strand of an insert. The required second amplification step of traditional method introduces a substantial amount of error in the sequencing reads obtained after the second amplification. The methods described herein, in certain embodiments, do not require a second amplification step and therefore provide for less error in the sequence reads obtained. Accordingly, in some embodiments, a method of sequencing both strands of a double stranded nucleic acid, as described herein, includes, or consists essentially of, generating a first read and a second read from the same template. In some embodiments, a method of sequencing both strands of a double stranded nucleic acid, as described herein, includes, or consists essentially of, generating a first read and a second read from a set of amplicons that are substantially complementary to a nucleic acid template. In some embodiments, a method of sequencing both strands of a double stranded nucleic acid, as described herein, includes, or consists essentially of, generating a first read and a second read from a set of amplicons that are substantially identical to a nucleic acid template.

In certain embodiments, a sequencing method provided herein includes sequencing both strands of a double stranded nucleic acid with an error rate of 5×10⁻⁵ or less, 1×10⁻⁵ or less, 5×10⁻⁶ or less, 1×10⁻⁶ or less, 5×10⁻⁷ or less, 1×10⁻⁷ or less, 5×10⁻⁸ or less, or 1×10⁻⁸ or less. In certain embodiments, a sequencing method provided herein includes sequencing both strands of a double stranded nucleic acid with an error rate of 5×10⁻⁵ to 1×10⁻⁸, 1×10⁻⁵ to 1×10⁻⁸, 5×10⁻⁵ to 1×10⁻⁷, 1×10⁻⁵ to 1×10⁻⁷, 5×10⁻⁶ to 1×10⁻⁸, or 1×10⁻⁶ to 1×10⁻⁸. In certain embodiments, a sequencing method provided herein includes sequencing both strands of a double stranded nucleic acid with an error rate of or 1×10⁻⁶ to 1×10⁻⁸. In certain embodiments, a sequencing method provided herein includes sequencing both strands of a double stranded nucleic acid with an error rate of 1×10⁻⁴ to 1×10⁻⁶. In certain embodiments, a sequencing method provided herein includes sequencing both strands of a double stranded nucleic acid with an error rate of 1×10⁻³ or less. In embodiments, a sequencing method provided herein includes sequencing both strands of a double stranded nucleic acid with an error rate of 1×10⁻⁴ or less. In embodiments, a sequencing method provided herein includes sequencing both strands of a double stranded nucleic acid with an error rate of 1×10⁻⁵ or less. In embodiments, a sequencing method provided herein includes sequencing both strands of a double stranded nucleic acid with an error rate of 1×10⁻⁶ or less. In embodiments, a sequencing method provided herein includes sequencing both strands of a double stranded nucleic acid with an error rate of 1×10⁻⁷ or less. In embodiments, a sequencing method provided herein includes sequencing both strands of a double stranded nucleic acid with an error rate of 1×10⁻⁸ or less.

Optionally, after obtaining sequences of both the first strand and second strand of the original double stranded insert from a single set of substantially identical amplicons (e.g., the first set of amplicons) attached to the substrate, a copy of each of the amplicons is generated by a process including annealing the free 3′-end of each amplicon to a surface-bound capture nucleic acid, extending the capture nucleic acid with a polymerase to generate third set of amplicons, removing the first set of amplicons from the substrate, and sequencing the third set of amplicons. In certain embodiments, the novel methods provided herein do not require this second amplification step which introduces additional error into the sequence reads obtained from the third set of amplicons.

In certain embodiments, templates or amplicons described herein are attached to addressable locations on a substrate using a suitable method known in the art or described herein.

In embodiments, a converted template nucleic acid is detected without sequencing. In embodiments, a converted template nucleic acid is detected through the use of a fluorescence-based, real-time PCR method, for example, MethyLight and Digital MethyLight, as described in Campan M. et al. Methods Mol Biol. 2018; 1708: 497-513, which is incorporated herein by reference. MethyLight relies on methylation-specific priming combined with methylation-specific fluorescent probing. Digital MethyLight involves distributing a MethyLight reaction across a 96- or 384-well plate or higher in a microfluidic device, such that the mean initial template DNA concentration is less than one molecule per reaction compartment. Amplification of methylated DNA molecules occurs in a small minority of PCR wells, and therefore represents a digital readout of the original number of template molecules in each sample.

In an aspect is provided a method of detecting a disease in a subject. In embodiments, the method includes obtaining a sample that includes a double-stranded nucleic acid from the subject; identifying whether a disease is present in the sample by sequencing the sample according to the methods described herein, and detecting a disease in a subject when the presence of a disease is identified in the sample. In another aspect is provided a method of diagnosing a subject with a disease. In embodiments, the method includes obtaining a sample that includes a double-stranded nucleic acid from the subject; identifying whether a disease is present in the sample by sequencing the sample according to the methods described herein, and diagnosing a subject with a disease when the presence of a disease is identified in the sample. In some embodiments, the disease is an autoimmune disease, hereditary disease, or cancer.

In embodiments, the disease is an autoimmune disease. In embodiments, the autoimmune disease is arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, or atopic dermatitis. In embodiments, the autoimmune disease is Achalasia, Addison's disease, Adult Still's disease, Agammaglobulinemia, Alopecia arcata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner car disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acnc Inversa), Hypogammalglobulinemia, IgA Nephropathy, IgG4-related sclerosing disease, Immune thrombocytopeniaurpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Parancoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, or Vogt-Koyanagi-Harada Disease.

In embodiments the disease is a hereditary disease. In embodiments, the hereditary disease is cystic fibrosis, alpha-thalassemia, beta-thalassemia, sickle cell anemia (sickle cell disease), Marfan syndrome, fragile X syndrome, Huntington's disease, or hemochromatosis.

In embodiments the disease is a cancer. As used herein, the term “cancer” refers to all types of cancer, neoplasm or malignant tumors found in mammals (e.g., humans), including leukemia, carcinomas and sarcomas. Exemplary cancers that may be treated with a compound or method provided herein include brain cancer, glioma, glioblastoma, neuroblastoma, prostate cancer, colorectal cancer, pancreatic cancer, cervical cancer, gastric cancer, ovarian cancer, lung cancer, and cancer of the head. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In embodiments, the cancer is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof. In embodiments, the cancer is a predefined stage of a breast cancer, a predefined stage of a lung cancer, a predefined stage of a prostate cancer, a predefined stage of a colorectal cancer, a predefined stage of a renal cancer, a predefined stage of a uterine cancer, a predefined stage of a pancreatic cancer, a predefined stage of a cancer of the esophagus, a predefined stage of a lymphoma, a predefined stage of a head/neck cancer, a predefined stage of a ovarian cancer, a predefined stage of a hepatobiliary cancer, a predefined stage of a melanoma, a predefined stage of a cervical cancer, a predefined stage of a multiple myeloma, a predefined stage of a leukemia, a predefined stage of a thyroid cancer, a predefined stage of a bladder cancer, or a predefined stage of a gastric cancer. In some embodiments, the cancer is a predefined subtype of a cancer. In certain instances, the cancer is early stage cancer. In other instances, the cancer is late stage cancer.

In embodiments, the subject is suspected of having a genetic variation or a disease or condition associated with a genetic variation (e.g., an oncogene). In embodiments, the sample, and/or the oncogene includes one or more mutations in one or more of the genes TP53, PIK3CA, PTEN, APC, VHL, KRAS, MLL3, MLL2, ARID1A, PBRM1, NAV3, EGFR, NF1, PIK3R1, CDKN2A, GATA3, RB1, NOTCHI, FBXW7, CTNNB1, DNMT3A, MAP3K1, FLT3, MALAT1, TSHZ3, KEAP1, CDH1, ARHGAP35, CTCF, NFE2L2, SETBP1, BAP1, NPM1, RUNX1, NRAS, IDH1, TBX3, MAP2K4, RPL22, STK11, CRIPAK, CEBPA, KDM6A, EPHA3, AKT1, STAG2, BRAF, AR, AJUBA, EPPKI, TSHZ2, PIK3CG, SOX9, ATM, CDKNIB, WTI, HGF, KDM5C, PRX, ERBB4, MTOR, TLR4, U2AF1, ARID5B, TET2, ATRX, MLL4, ELF3, BRCA1, LRRK2, POLQ, FOXA1, IDH2, CHEK2, KIT, HIST1H1C, SETD2, PDGFRA, EP300, FGFR2, CCND1, EPHB6, SMAD4, FOXA2, USP9X, BRCA2, NFE2L3, FGFR3, ASXLI, TGFBR2, SOX17, CDKN1A, B4GALT3, SF3B1, TAFI, PPP2R1A, CBFB, ATR, SIN3A, VEZF1, HIST1H2BD, EIF4A2, CDK12, PHF6, SMC1A, PTPN11, ACVRIB, MAPK8IP1, H3F3C, NSD1, TBL1XR1, EGR3, ACVR2A, MECOM, LIFR, SMC3, NCOR1, RPL5, SMAD2, SPOP, AXIN2, MIR142, RAD21, ERCC2, CDKN2C, EZH2, or PCBP1. In embodiments, the cancer is lung cancer, colorectal cancer, skin cancer, colon cancer, pancreatic cancer, breast cancer, cervical cancer, lymphoma, leukemia, or a cancer associated with aberrant K-Ras, aberrant APC, aberrant Smad4, aberrant p53, or aberrant TGFβ. In embodiments, the cancer cell includes a ERBB2, KRAS, TP53, PIK3CA, or FGFR2 genc.

### EXAMPLES

### Example 1: Linked Duplex Methylation Profiling

It is a major challenge to distinguish true variants from background noise for rare variant discovery. Typically, large amounts of sequencing data (high sequencing depth) are required to differentiate a true variant from an amplification and/or sequencing error using previous methods. In addition, the heterogeneity of mutations within populations of rare cells in liquid biopsy samples, such as plasma or saliva, make it difficult to distinguish between sequencing-related errors and true somatic mutations originating from tumors. Methods and constructs provided herein do not necessarily require such large sequencing depths, thereby dramatically reducing the costs associated with sequencing.

Nucleic acids, e.g., cell-free DNA (cfDNA), are released into the bloodstream and other body fluids as part of natural cell apoptosis, necrosis, and secretion, and includes both single- and double-stranded DNA fragments that are relatively short (overwhelmingly less than 200 base-pairs) and are normally at a low concentration (e.g. 1-100 ng/mL in plasma). It is known that the concentration of cfDNA and ctDNA (circulating tumor DNA) in plasma correlates with tumor size and stage. For example, patients having stage I cancer types had fewer than 10 copies per 5 ml of tumor mutations in plasma. In contrast, the copy number increased 10 to 100 times among late-stage patients (Haque et al. bioRxiv. 2017;237578). Thus, ctDNA assays used for early cancer diagnoses should be highly sensitive. Commercial solutions require UMIs on both strands of the double-stranded template, followed by low-error sequencing. To determine a true variant using previous commercial solutions, a large amount of sequencing data (high sequencing depth) is required to generate a consensus sequencing read to confidently ascertain a single nucleotide change.

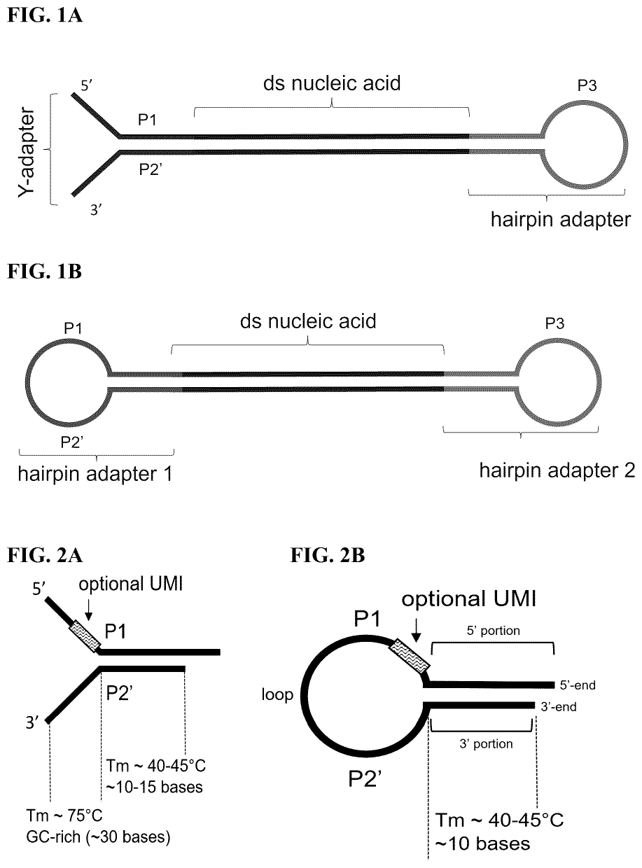

To address these issues, we designed a protocol described herein for ligating two different adapters at each end of a double stranded template nucleic acid (see, e.g., FIG. 1). High accuracy sequencing reads would be particularly useful for rare variant detection in cfDNA. DNA variants cannot be statistically distinguished from sample prep and/or sequencing errors when they are present within a sample at a frequency below the error rate of the sequencing method (typically between 0.05-1%). By linking the parent DNA strands together with a hairpin adapter as described herein, and sequencing both strands of the double stranded template nucleic acid, sample prep and sequencing errors can be distinguished from true variants by identifying discordant base calls between the complementary sequencing reads. True genetic variants will be observed as mutations on both strands, whereas errors will only be observed as mutations on a single strand. The sequencing method described herein allows for very high accuracy results by identifying and correcting for errors within the data which in turn increases the sensitivity for rare variant detection. In general, since two independent sequencing measurements are made for the same base, that is, one for each strand, the accuracy of the consensus base call is the product of the error rate for each individual base call. For example, if the single-pass error rate is 10⁻³ (Q30), then pairwise sequencing using the methods described allows for a double pass rate to be at least double the single-pass rate, i.e., 10⁻⁶ (Q60).

Beyond somatic mutations, epigenetic information, such as biomolecule methylation, and/or additional protein biomarkers combined with cfDNA and ctDNA analyses are useful in determining tumor origin at an early stage. Biomolecule methylation, such as DNA methylation, is widespread and plays a critical role in the regulation of gene expression in development, differentiation, and disease. Methylation is an epigenetic modification in which a methyl group is added to cytosines and/or adenine nucleobases, and frequently occurs in regions of DNA where a cytosine nucleotide is followed by a guanine nucleotide in the linear sequence of bases along its 5′->3′ direction, referred to as a CG or CpG site. In particular regions of genes, for example gene promoter regions, an increase in cytosine methylation at gene promotor regions can inhibit the expression of these genes (Robertson K D Nat. Rev. Genet. 6, 597-610 (2005)). The gene silencing effect of methylated regions is accomplished through the interaction of methylcytosine binding proteins with other structural components of the chromatin, which, in turn, makes the DNA inaccessible to transcription factors through histone deacetylation and chromatin structure changes (Greenberg M V C and Bourc'his D Nat. Rev. Mol. Cell Biol. 20, 590-607 (2019)). Cancers take advantage of this mechanism, and hypermethylate genomic regions associated with DNA repair genes.

Methylation patterns also play an important role in genomic imprinting, in which imprinted genes are preferentially expressed from either the maternal or paternal allele. Patterns of methylation in a genome are heritable because of the semi-conservative nature of DNA replication. During this process, the daughter strand, newly replicated on a methylated template strand is not initially methylated, but the template strand directs methyltransferase enzymes to fully methylate both strands. Deregulation of imprinting has been implicated in several developmental disorders. Moreover, there is abundant evidence that aberrant DNA methylation can preclude normal development.

There are around 25,000 CpG islands in the human genome. CpG islands are usually understood as polynucleotide regions with a length greater than 200 bp having GC content greater than 50%. In various cancers such as leukemia, it has been previously reported that there is a global decrease in DNA methylation and an increase in methylation specifically at CpG islands. It is believed that in a normal cell, the CpG islands are unmethylated and when the cell becomes a tumor cell the CpG island becomes methylated at every CpG. It is suspected that in a normal cell the CpG islands, which are typically located near the promoters of genes, are normally kept hypomethylated. In an unmethylated state, cytosine is converted to uracil after deamination, which is recognized by the cell's repair machinery and is removed, while in a methylated state deamination of cytosine results in the formation of thymine which is not recognized by the repair machinery. Therefore, the presence or absence of hypermethylation at these CpG islands can be used to detect tumor cells. As cancer cells are constantly evolving to avoid treatment regimens, there is a need for a method to detect a tumor cell with high accuracy.

A common method of determining the methylation level and/or pattern of DNA requires methylation status-dependent conversion of cytosine in order to distinguish between methylated and non-methylated CpG dinucleotide sequences. For example, bisulfite conversion is a process in which genomic DNA is denatured (i.e., rendered single-stranded) and treated with sodium bisulfite, leading to deamination of unmethylated cytosine nucleobases into uracil nucleobases, while methylated cytosine nucleobases (e.g., 5-methylcytosine and 5-hydroxymethylcytosine) remain unchanged. Thus, thymine nucleobases detected in bisulfite sequencing correspond to either thymine nucleobases or unmethylated cytosine nucleobases in the original DNA, and alignment with the original template sequence easily differentiates between them. One consequence of bisulfite-mediated deamination of cytosine is that the bisulfite treated cytosine is converted to uracil, which reduces the complexity of the genome. Specifically, a typical 4-base genome (A, T, C, G) is essentially reduced to a 3-base genome (A, T, G) because uracil is read as thymine during downstream analysis techniques such as PCR and sequencing reactions. Thus, the only cytosines present are those that were methylated prior to bisulfite conversion. Commercial solutions for generating bisulfite-converted libraries are available and include, for example, the xGen™ Methylation-Sequencing DNA Library Preparation Kit (IDT catalog #10009860). Because the complexity of the genome is reduced, standard methods for comparing and/or aligning a bisulfite-converted sequence to the pre-conversion genome can be cumbersome and, in some cases, ineffective. For example, problems may arise when aligning converted fragments to the genome, especially when using relatively short sequences (e.g., 25 to 75 bp).

While bisulfite conversion is the current standard for performing DNA methylation analysis, it is a harsh chemical reaction which can lead to DNA degradation, severely limiting its utility if sample DNA quantities are low, as is often the case with cfDNA. Additionally, the complete conversion of unmodified cytosine to thymine reduces sequencing complexity, potentially leading to poor sequencing quality, low mapping rates, and uneven genome coverage. An alternative method for bisulfite-free detection of modified cytosine nucleobases (e.g., 5-methylcytosine (5mC) and 5-hydroxymethylcytosine (5hmC)) has been described (Liu Y et al. Nat. Biotechnol. 2019, 37(4)424-429, which is incorporated herein by reference), which utilizes an enzymatic approach, and combines the ten-eleven translocation (TET) oxidation of 5mC and 5hmC to 5-carboxylcytosine (5caC) with pyridine borane reduction of 5caC to dihydrouracil (DHU). Subsequent PCR converts DHU to thymine, enabling a C-to-T transition of 5mC and 5hmC. This TET-assisted pyridine borane sequencing (TAPS) method results in higher mapping rates and more even coverage than bisulfite conversion and may be applied to the methods described herein for linked duplex methylation profiling. Another bisulfite-free approach for methylation analysis is the NEBNext® Enzymatic Methyl-seq product (EM-Seq), which first protects 5mC and 5hmC from deamination by TET2 and an oxidation enhancer, followed by APOBEC deamination of unprotected cytosines to uracils. However, these enzymatic methods require single stranded nucleic acids (i.e., denaturing the double stranded template nucleic acid). As described above, it is advantageous to retain the double stranded information of the original double stranded template nucleic acid.

Provided herein are methods and compositions that relate to sequencing nucleic acids and determining the methylation level and/or pattern of the nucleic acids. Using methods and/or compositions described herein, the complexity of the target nucleic acids is preserved by keeping track of complementary strands after the strands have been subjected to bisulfite conversion of nucleic acids. In order to preserve complexity of the nucleic acid, embodiments of the present invention relate to a pairing of the cytosine-converted sequences of both strands of a double-stranded nucleic acid and using the sequence information from both strands to determine the sequence and/or methylation status of one or both strands prior to conversion.

### Example 2. Linked Duplex Sequencing: Methylated/Unmethylated Cytosine Conversion

Methylation of CpG dinucleotide sequences can be measured by employing cytosine conversion-based technologies. Commonly used bisulfite conversion modifies unmethylated cytosine nucleobases to uracil nucleobases. Sodium bisulfite (NaHSO₃) reacts readily with the 5,6-double bond of cytosine, but poorly with methylated cytosine, as described by Olek A., Nucleic Acids Res. 24:5064-6, 1996 or Frommer et al., Proc. Natl. Acad. Sci. USA 89:1827-1831 (1992), each of which is incorporated herein by reference. Cytosine reacts with the bisulfite ion to form a sulfonated cytosine reaction intermediate which is susceptible to deamination, giving rise to a sulfonated uracil. The sulfonate group can be removed under alkaline conditions, resulting in the formation of uracil. Uracil is recognized as a thymine by Taq polymerase and other polymerases and therefore upon PCR or during a sequencing reaction, the resultant product contains cytosine only at the position where 5-methylcytosine occurs in the starting template nucleic acid (see, e.g., FIG. 4A). Alternatively, conversion may be accomplished using restriction enzymes, such as HpaII and MspI, which recognize the sequence CCGG.

Traditionally, the recovery of bisulfite-converted DNA is limited due to DNA degradation caused by extended-duration sodium bisulfite treatment protocols and subsequent depyrimidation (Grunau C et al. Nucleic Acids Res. 2001, 29(13):E65-5). Optimized bisulfite conversion protocols that include a fast deamination step reduce incubation times from 12 to 16 hours to 40 min by using a highly concentrated bisulfite solution at high temperatures, leading to a more homogenous conversion of cytosine due to the casier process of DNA denaturation at high temperatures and reduced degradation due to shorter incubation times (Shiraishi M and Hayatsu H. DNA Res. 2004, 11(6):409-15). One study has shown that bisulfite treatment of cfDNA for 30 min at 70° C. leads to complete conversion of cytosine to uracil and is achieved with high post-treatment DNA recovery (Yi S et al. BMC Molecular Biol. 2017, 18:24, which is incorporated herein by reference). Such rapid-bisulfite conversion can also be used in the method described herein. For example, 10 M(NH₄)HSO₃—NaHSO₃ bisulfite solution was added to Y-template-hairpin constructs. The mixtures are heated for 30 min at 70° C. or for 10 min at 90° C. and subsequently cooled to 4° C.

### Linked Duplex Sequencing: Library Prep Overview

Commercially available next-generation sequencing (NGS) technologies typically require library preparation, whereby a pair of specific adapter sequences are ligated to the ends of DNA fragments in order to enable sequencing by the instrument. Typically, preparation of a nucleic acid library involves 5 steps: DNA fragmentation, polishing, adapter ligation, size selection, and library amplification.

Fragmentation of DNA can be achieved by enzymatic digestion or physical methods (e.g., sonication, nebulization, or hydrodynamic shearing). Enzymatic digestion produces DNA ends that can be efficiently polished and ligated to adapter sequences. However, it is difficult to control the enzymatic reaction and produce fragments of predictable length. In addition, enzymatic fragmentation is frequently base-specific thus introducing representation bias into the sequence analysis. Alternatively, physical methods to fragment DNA are random and DNA size distribution can be more easily controlled, but DNA ends produced by physical fragmentation are often damaged and a conventional polishing reaction may be insufficient to generate ample ligation-compatible ends. Typical polishing mixtures contain T4 DNA polymerase and T4 polynucleotide kinase. These enzymes excise 3′ overhangs, fill in 3′ recessed ends, and remove any potentially damaged nucleotides thereby generating blunt ends on the nucleic acid fragments. The T4 polynucleotide kinase used in the polishing mix adds a phosphate to the 5′ ends of DNA fragments that can be lacking such, thus making them ligation-compatible to NGS adapters.

Prior to ligation, adenylation of repaired nucleic acids using a polymerase which lacks 3′-5′ exonuclease activity is often performed in order to minimize chimera formation and adapter-adapter (dimer) ligation products. In these methods, single 3′ A-overhang DNA fragments are ligated to single 5′ T-overhang adapters, whereas A-overhang fragments and T-overhang adapters have incompatible cohesive ends for self-ligation. During size selection, fragments of undesired sizes are eliminated from the library using gel or bead-based selection in order to optimize the library insert size for the desired sequencing read length. This often maximizes sequence data output by minimizing overlap of paired end sequencing that occurs from short DNA library inserts. Amplifying libraries prior to NGS analysis is typically a beneficial step to ensure there is a sufficient quantity of material to be sequenced.

### Linked Duplex Sequencing: Ligating Adapters

In some aspects of a method herein, an adapter-target-adapter nucleic acid template (FIG. 1A and FIG. 1B) is provided where two non-identical adapters are ligated to each respective end of a polynucleotide duplex. Embodiments of adapters contemplated herein include those shown in FIGS. 2A-2D. A polynucleotide duplex refers to a double-stranded portion of a polynucleotide, for example a polynucleotide desired to be sequenced.

As depicted in FIG. 1A, a first adapter is a Y adapter (alternatively, this may be referred to as a mismatched adapter or a forked adapter) that is ligated to one end of a polynucleotide duplex. The adapter is formed by annealing two single-stranded oligonucleotides, herein referred to as P1 and P2′. P1 and P2′ may be prepared by a suitable automated oligonucleotide synthesis technique. The oligonucleotides are partially complementary such that a 3′ end and/or a 3′ portion of P1 is complementary to the 5′ end and/or a 5′ portion of P2′. A 5′ end and/or a 5′ portion of P1 and a 3′ end and/or a 3′ portion of P2′ are not complementary to each other, in certain embodiments. When the two strands are annealed, the resulting Y adapter is double-stranded at one end (the double-stranded region) and single-stranded at the other end (the unmatched region), and resembles a ‘Y’ shape.

The single-stranded portions (the unmatched regions) of both P1 and P2′ have an elevated melting temperature (Tm) (e.g., about 75° C.) relative to their respective complements to enable efficient binding of surface primers and stable binding of sequencing primers. To achieve an elevated Tm in a reasonable length primer, the GC content is often >50% (e.g., approximately 60-75% GC content). In contrast to the single-stranded portions, a double-stranded region, in certain embodiments, has a moderate Tm (e.g., 40-45° C.) so that it is stable during ligation. In embodiments, a double-stranded region has an elevated Tm (e.g., 60-70° C.). In embodiments, the GC content of the double-stranded region is >50% (e.g., approximately 60-75% GC content). The unmatched region of P1 and P2′, in certain embodiments, are about 25-35 nucleotides (e.g., 30 nucleotides), whereas the double-stranded region is shorter, ranging about 10-20 nucleotides (e.g., 13 nucleotides) in total. For example, P2′ may be a total of 43 nucleotides in length, as shown in FIG. 2A. In embodiments, the P1 region of the Y adapter has the sequence S1 sequence (SEQ ID NO: 1) and the P2′ region of the Y adapter has the S2 (SEQ ID NO:3) sequence, as described in Table 1 below. In embodiments, the P1 region of the Y adapter has the sequence S4 sequence (SEQ ID NO:2) and the P2′ region of the Y adapter has the S5 (SEQ ID NO:4) sequence, as described in Table 1 below.

TABLE 1
Sequences for the Y adapters. Note, the ‘*’ is indicative of an
optional phosphorothioate linkage. Phosphorothioate linkages assist
in protecting the oligonucleotide against exonuclease degradation
from certain polymerases (e.g., phi29).
P1 regions of the Y adapter

| | |
|---|---|
| S1 (SEQ ID NO: 1) | /5Phos/ACAAAGGCAGCCACGCACTCCTTCC |
| | CTGAAGGCCGGAATC*T |
| S4 (SEQ ID NO: 2) | /5Phos/GCTGCCGCCACTAGCCATCTTACTG |
| | CTGAGGACTCTTCGC*T |

P2′ regions of the Y adapter

| | |
|---|---|
| S2 (SEQ ID NO: 3) | /5Phos/GATTCCGGCCTTGTGGTTGGTGAGG |
| | GTCATCTCGCTGGAG |
| S5 (SEQ ID NO: 4) | /5Phos/GCGAAGAGTCCTGGAGTGCCGCCAA |
| | TGTATGCGAGGGTGA |

As shown in FIG. 2D, the double-stranded region of the forked adapter may be blunt-ended (top), it may have a 3′ overhang (middle), or a 5′ overhang (bottom). The overhang may include a single nucleotide or more than one nucleotide. The 5′ end of the double-stranded part of the forked adapter is phosphorylated, i.e., the 5′ end of P2′. The presence of the 5′ phosphate group (referred to as 5′P in FIG. 2D) allows the adapter to ligate to the polynucleotide duplex. The 5′ end of P1 may be biotinylated or have a functional group at the end, thus enabling it to be immobilized on a surface (e.g., a planar solid support).

Alternatively, as depicted in FIG. 1B, the first adapter is a hairpin adapter (e.g., the hairpin adapter of FIG. 2B) and it is ligated to one end of a polynucleotide duplex.

The second adapter is a hairpin adapter (alternatively, it may be referred to as a stem-loop adapter, barbell, or hairpin loop adapter) and it is ligated to one end of a polynucleotide duplex, depicted as containing a P3 priming site in FIG. 1B and FIG. 2C. The hairpin adapter includes a double-stranded region which has a moderate Tm (e.g., 40-45° C.) so that it is stable during ligation, and includes at least 10 nucleotides. The hairpin adapter also includes a loop region which has a primer sequence and has an elevated Tm (e.g., 75° C.) relative to the double stranded region to enable stable binding of a complementary sequencing primer. The loop region or the stem region of the hairpin may further include a barcode or Unique Molecular Identifier (UMI) using degenerate sequences. The UMI consists of 3-5 degenerate nucleotides.

TABLE 2
Sequences for the hairpin adapter. Note, the ‘*’ is indicative
of an optional phosphorothioate linkage. Phosphorothioate linkages assist in
protecting the oligonucleotide against exonuclease degradation from certain
polymerases (e.g., phi29).

| | |
|---|---|
| B1 (SEQ ID NO: 5) | /5Phos/GCGCGCG TTT TTT TT |
| | GCTTGCGTCTCCTGCCAGCCATATCCGGTCTACGTGATCC TTT TTT TT |
| | CGCGCGC*T |
| B2 (SEQ ID NO: 6) | /5Phos/GCGCGCGTTT TTT TTT TTT TT |
| | GCTTGCGTCTCCTGCCAGCCATATCCGGTCTACGTGATCC TTT TTT |
| | TTT TTT TT CGCGCGC*T |
| B3 (SEQ ID NO: 7) | /5Phos/GGATCACGTAGATTTTGCTTGCGTCTCCTGCCAGCCATATCCG |
| | GTTTTTCTACGTGATTCC*T |
| B4 (SEQ ID NO: 8) | /5Phos/GCGAAGAGTCCT GGAGTGCCGCCAATGTATGCGAGGGTGA |
| S4S5′_in loop_0Ts | GCTGCCGCCACTAGCCATCTTACTGCTG AGGACTCTTCGC*T |
| B5 (SEQ ID NO: 9) | /5Phos/GCGAAGAGTCCT TTT TTT |
| S4S5′_in loop_6Ts | GGAGTGCCGCCAATGTATGCGAGGGTGA |
| | GCTGCCGCCACTAGCCATCTTACTGCTG TTT TTT AGGACTCTTCGC*T |
| B6 (SEQ ID NO: 10) | /5Phos/GCGAAGAGTCCT TTT TTT |
| S4S5′_in loop_6 + | GGAGTGCCGCCAATGTATGCGAGGGTGA TTT TTT T |
| 8Ts | GCTGCCGCCACTAGCCATCTTACTGCTG TTT TTT AGGACTCTTCGC*T |
| B7 (SEQ ID NO: 11) | /5Phos/GATTCCGGCCTT |
| S1S2′_in loop_0Ts | GTGGTTGGTGAGGGTCATCTCGCTGGAGACAAAGGCAGCCACGCACTCC |
| | TTCCCTGAAGGCCGGAATC*T |
| B8 (SEQ ID NO: 12) | /5Phos/GATTCCGGCCTT TTT TTT |
| S1S2′_in loop_6Ts | GTGGTTGGTGAGGGTCATCTCGCTGGAGACAAAGGCAGCCACGCACTCC |
| | TTCCCTG TTTTTT AAGGCCGGAATC*T |
| B9 (SEQ ID NO: 13) | /5Phos/GATTCCGGCCTT TTT TTT |
| S1S2′_in loop_6 + | GTGGTTGGTGAGGGTCATCTCGCTGGAGTTT TTT |
| 7Ts | TACAAAGGCAGCCACGCACTCCTTCCCTG TTT TTT |
| | AAGGCCGGAATC*T |
| B10 (SEQ ID NO: 14) | /5Phos/GGATCACGTAGATTTTGCTTGCGTCTCCTGCCAGCCATATCCG |
| | GTTTTTCTACGTGATCC*T |
| B11 (SEQ ID NO: 15) | 5Phos/GG ATC ACG TAG ATT TTT TTT TTT TGC TTG CGT CTC CTG |
| B10 + 12T | CCA GCC ATA TCC GGT TTT TTT TTT TTT CTA CGT GAT CC*T |
| B12 (SEQ ID NO: 16) | 5Phos/GG ATC ACG TAG ATT TTT TTT TTT TTT TTT TTT TTT TGC |
| B10 + 24T | TTG CGT CTC CTG CCA GCC ATA TCC GGT TTT TTT TTT TTT TTT |
| | TTT TTT TTT CTA CGT GAT CC*T |
| B13 (SEQ ID NO: 17) | 5Phos/GG ATC ACG TAG ATT TTT TTT TTT TTT TTT TTT TTT |
| B10 + 40-10T | TTT TTT TTT TTT TTT TTG CTT GCG TCT CCT GCC AGC CAT ATC |
| | CGG TTT TTT TTT TTC TAC GTG ATC C*T |
| B14 (SEQ ID NO: 18) | /5Phos/GGA TCA CGT AGA TTT TAG ATC TGC TTG CGT CTC CTG CCA |
| B10 + clv | GCC ATA TCC GGT TTT TCT ACG TGA TCC* T |
| B15 (SEQ ID NO: 19) | /5Phos/GGA TCA CGT AGA TTTTTTTTTTTT AGA TCT GCT TGC GTC |
| B11 + clv | TCC TGC CAG CCA TAT CCG GTTTTTTTTTTTTC TAC GTG ATC C*T |

In embodiments, a hairpin adapter includes a sequence selected from SEQ ID NOs:5-17. In embodiments, the hairpin adapter has the B1 (SEQ ID NO:5) sequence described in Table 2. In embodiments, the hairpin adapter has the B2 (SEQ ID NO:6) sequence described in Table 2. In embodiments, the hairpin adapter has the B3 (SEQ ID NO:7) sequence described in Table 2. In embodiments, the hairpin adapter has the B4 (SEQ ID NO:8) sequence described in Table 2. In embodiments, the hairpin adapter has the B5 (SEQ ID NO:9) sequence described in Table 2. In embodiments, the hairpin adapter has the B6 (SEQ ID NO:10) sequence described in Table 2. In embodiments, the hairpin adapter has the B7 (SEQ ID NO:11) sequence described in Table 2. In embodiments, the hairpin adapter has the B8 (SEQ ID NO:12) sequence described in Table 2. In embodiments, the hairpin adapter has the B9 (SEQ ID NO:13) sequence described in Table 2. In embodiments, the hairpin adapter has the B10 (SEQ ID NO:14) sequence described in Table 2. In embodiments, the hairpin adapter has the B11 (SEQ ID NO: 15) sequence described in Table 2. In embodiments, the hairpin adapter has the B12 (SEQ ID NO:16) sequence described in Table 2. In embodiments, the hairpin adapter has the B13 (SEQ ID NO: 17) sequence described in Table 2. In embodiments, the hairpin adapter has the B13 (SEQ ID NO: 18) sequence described in Table 2. In embodiments, the hairpin adapter has the B13 (SEQ ID NO: 19) sequence described in Table 2.

As shown in FIG. 2D, the double-stranded region of the hairpin adapter may be blunt-ended (top), it may have a 5′ overhang (middle), or a 3′ overhang (bottom). The overhang may include a single nucleotide or more than one nucleotide. The 5′ end of the double-stranded part of the hairpin adapter is phosphorylated. The presence of the 5′ phosphate group allows the adapter to ligate to the polynucleotide duplex.

The order of ligation events is not relevant, however for the purposes of discussion the terms ‘first’ and ‘second’ are used in reference to the sequence in which the adapter is ligated to the polynucleotide duplex. It is understood that the ligation of the Y adapter or the hairpin adapter may occur first, such that the resulting adapter-target-adapter constructs contain non-identical adapters.

Note, during this step it is possible to form adapter dimers (i.e., two adapters ligate together with no intervening template nucleic acid). There are several ways to reduce adapter dimer formation in the adapter ligation NGS library preparation described herein, including i) a stringent purification step (e.g., SPRI) after 3′ adapter ligation to remove non-ligated 3′ adapter molecules, prior to the second ligation of the 5′ adapter; ii) the use of A-tailed DNA and T-overhang adapters; iii) or utilizing alkaline phosphatase treatment after 3′ adapter ligation, before any SPRI cleanup, to remove 5′ phosphate group from the 3′ adapter to render any carryover 3′ adapter to be ligation incompatible and inert in the 5′ adapter ligation step.

### Methods

Fragmented DNA may be made blunt-ended by a number of methods known to those skilled in the art. In embodiments, the ends of the fragmented DNA are end repaired with T4 DNA polymerase and Klenow polymerase, a procedure well known to those skilled in the art, and then phosphorylated with a polynucleotide kinase enzyme. A single ‘A’ deoxynucleotide is then added to both 3′ ends of the DNA molecules using Taq polymerase enzyme, producing a one-base 3′ overhang that is complementary to the one-base T overhang on the double-stranded end of the Y adapter and hairpin adapter. For example, in the presence of a T4 DNA ligase, an A overhang is created on both strands at the 3′ hydroxyl end of a target duplex polynucleotide. For example, using Blunt/TA Ligase Master Mix (NEB #M0367) includes a T4 DNA ligase in a reaction buffer and ligation enhancers to ensure efficient A tailing. It is preferable to polish or use a filling reaction to ensure the ends of the target duplex polynucleotide are blunt before adding the A overhang. Examples of ends that need polishing or filling include inserts generated by shearing or sonication. A number of DNA polymerases will remove DNA overhangs and/or can be used to fill in missing bases if there is a 3′ hydroxyl available for priming. Polymerases for such reactions include, but are not limited to, a T4 DNA polymerase, PFU, and the Klenow Fragment of DNA polymerase I.

A ligation reaction between the Y adapter, the hairpin adapter, and the DNA fragments is then performed using a suitable ligase enzyme (e.g. T4 DNA ligase) which joins one hairpin adapter and one Y adapter to each DNA fragment, one at either end, to form adapter-target-adapter constructs that somewhat resemble a bobby pin hair fastener (see FIG. 1A). Alternatively, a ligation reaction between a first hairpin adapter (e.g., FIG. 2B), and a different second hairpin adapter (e.g., FIG. 2C), and the DNA fragments is then performed using a suitable ligase enzyme (e.g., T4 DNA ligase) which joins the first hairpin adapter and the second hairpin adapter to each DNA fragment, one at either end, to form adapter-target-adapter constructs (see FIG. 1B).

The products of this reaction can be purified from leftover unligated adapters that by a number of means (e.g., NucleoMag NGS Clean-up and Size Select kit, Solid Phase Reversible Immobilization (SPRI) bead methods such as AMPureXP beads, PCRclean-dx kit, Axygen AxyPrep FragmentSelect-I Kit), including size-inclusion chromatography, preferably by electrophoresis through an agarose gel slab followed by excision of a portion of the agarose that contains the DNA greater in size that the size of the adapter.

### Linked Duplex Sequencing: Methylated/Unmethylated Cytosine Conversion

While bisulfite conversion is the current standard for performing DNA methylation analysis, it has several drawbacks. As discussed supra, bisulfite treatment is a harsh chemical reaction which can lead to DNA degradation, severely limiting its utility if sample DNA quantities are low, as is often the case with cfDNA. Additionally, the complete conversion of unmodified cytosine to thymine reduces sequencing complexity, potentially leading to poor sequencing quality, low mapping rates, and uneven genome coverage. A method for bisulfite-free direct detection of 5-methylcytosine (5mC) and 5-hydroxymethylcytosine (5hmC) has been described (Liu Y et al. Nat. Biotechnol. 2019; 37(4):424-429, which is incorporated herein by reference), which combines ten-eleven translocation (TET) oxidation of 5mC and 5hmC to 5-carboxylcytosine (5caC) with pyridine borane reduction of 5caC to dihydrouracil (DHU) (see, e.g., FIG. 3B and FIG. 4B). Subsequent PCR converts DHU to thymine, enabling a C-to-T transition of 5mC and 5hmC. This TET-assisted pyridine borane sequencing (TAPS) method results in higher mapping rates and more even coverage than bisulfite conversion and may be applied to the methods described herein for linked duplex methylation profiling. Another bisulfite-free approach for methylation analysis is the NEBNext® Enzymatic Methyl-seq (EM-seq) product, which first protects 5mC and 5hmC from deamination by TET2 and an oxidation enhancer, followed by APOBEC deamination of unprotected cytosines to uracils (see, e.g., FIG. 4C).

Converted DNA can subsequently be analyzed by conventional molecular techniques, such as PCR amplification, sequencing, and detection including oligonucleotide hybridization. As described below, a variety of techniques are available for sequence-specific analysis (e.g., MSP) of the methylation status of one or more CpG dinucleotides in a particular region of interest. Methods provided herein are particularly useful for creating a reference complimentary copy of the pre-conversion sequence for each of a multitude of genomic fragments. Using these methods, the reference copy may be covalently linked to the converted template. By linking parent strands together using the constructs described herein (e.g., the hairpin adapter depicted in FIG. 2C), the sequence can be corrected prior to mapping using the second strand, increasing the fraction of properly mapped reads. Additionally, C to T mutations (SNVs) are distinguishable from converted bases as the “T” mutation will be confirmed by an “A” on the opposite strand enabling both detection of sequencing variants and methylation state in the same assay.

The DNA is fragmented, repaired, and adapters ligated as described supra. As a result of cytosine conversion, unmethylated cytosines in the template nucleic acid are converted to uracil residues, while methylated cytosines are unchanged (see, for example, FIG. 3A). In embodiments, the cytosine-converted construct may be amplified prior to hybridization to increase the amount of material available for cluster amplification, resulting in conversion of the uracil nucleotides (dUTP) to thymine nucleotides (dTTP). In embodiments where the adapter oligonucleotides include a sequence that will be used in later steps (i.e., for capture on a support or for binding of a sequencing primer), the adapter can be synthesized, for example, using a bisulfite-resistant cytosine analog such as 5-methyl dCTP (Me-C, or 5mC) in the positions where maintaining a cytosine at that position is important. Alternatively, a hairpin adapter could be ligated to one side of a linear template, with the hairpin adapter functioning as a primer to fill in the second strand of the template with dNTPs including Me-C. Following cytosine conversion, the second strand remains unconverted due to the incorporated Me-C bases and can serve as a reference for the original converted template strand.

Described herein, the methods use the physical pairing of the complementary strands to identify DNA fragments having an asymmetric methylcytosine profile (e.g., hemimethylated DNA fragments). These can arise from imprinting, but also as a consequence of active demethylation catalyzed by TET family enzymes (Erlich et al 2012, Shen et al 2014, Song et al 2017), which are misregulated in some cancers. During TET mediated active demethylation, the standard methyl cytosine is converted to 5-hydroxymethylcytosine as well as additional intermediates, finally resulting in an unmethylated cytosine. 5-hydroxymethylcytosine and other intermediates are relatively short lived and are found at low frequency in a cell type undergoing active demethylation.

Quantifying 5-hydroxymethylcytosine and these additional intermediates as a liquid biopsy biomarker is helpful at obtaining an epigenetic snapshot of the cancer status. The methyl moiety of methylated cytosines can be lost or eliminated e.g., passively during DNA replication, or actively through enzymatic DNA demethylation. During active DNA demethylation, 5mC is oxidized to produce 5-hydroxymethylcytosine (5hmC), which acts not only as an intermediate during 5mC demethylation, but also plays important roles in many cellular and developmental processes, including the pluripotency of embryonic stem cells, neuron development, and tumorigenesis in mammals. Methods described herein are useful at quantifying 5mC and 5hmC in both strands of a sample and are useful at revealing the extent of methylation symmetry in a double-stranded nucleic acid. For example, using the methods described herein, one method to detect asymmetric methylation profiles consists of (1) ligating a first adapter and a second adapter to a cfDNA sample, wherein the second adapter is a hairpin adapter, wherein some or all of the adapter cytosines of the hairpin adapters are methylated; (2) optionally, capturing fragments using a hybrid capture panel; (3) converting the cytosines (e.g., contacting the sample with bisulfite to treat the fragments, or using an enzymatic conversion methodology); (4) amplifying the converted sample; and (5) sequencing to identify forward and reverse read mismatches indicative of asymmetric methylation. One useful embodiment of the above approach employs hairpin adapters designed to contain a region consisting of partially methylated cytosines (e.g., during hairpin oligomer synthesis, request that a given position consist of an equal proportion of cytosines and methylcytosines). Bisulfite conversion provides information on the methylation state of individual cytosines by converting cytosine (but not 5-methylcytosine) to uracil, and subsequently to thymine upon PCR amplification. Following bisulfite treatment, a subset of the adapter cytosines would undergo conversion and be read as thymine. The resultant random 2-base code gives rise to a low complexity “methylcytosine UMI” for use in downstream error correction. Hairpin adapters could be further improved, in some embodiments, by inclusion of a “bisulfite conversion control region” consisting of one or more unmethylated cytosines, which undergo bisulfite conversion and are read as thymines. Quantifying the fraction of unconverted cytosines bases in this region provides an indication of the efficiency of bisulfite conversion and may serve as a quality control metric.

Linked Duplex Sequencing: Methylated/Unmethylated Cytosine Conversion with Blocking Strand

Methods for cytosine conversion exhibit preferential activity towards single-stranded DNA, and typically require denaturation of a double-stranded template prior to performing a conversion reaction (e.g., chemical and/or enzymatic conversion). In a typical cytosine conversion protocol, a double-stranded template is melted into single-stranded polynucleotides, and each strand is subjected to the cytosine conversion method being performed. Described herein is a method that takes advantage of the bobby-pin fastener shape (i.e., a Y-shaped adapter on one end of a template and a hairpin adapter on a second end of the template) to generate a template nucleic acid with converted cytosines present on only one strand. This method provides an unmodified polynucleotide strand in the linked complementary strand of the template nucleic acid following the cytosine conversion protocol, providing an internal reference for modifications present on each template nucleic acid molecule that can subsequently be sequenced, simultaneously increasing sequencing depth and accurate detection of nucleobase modifications.

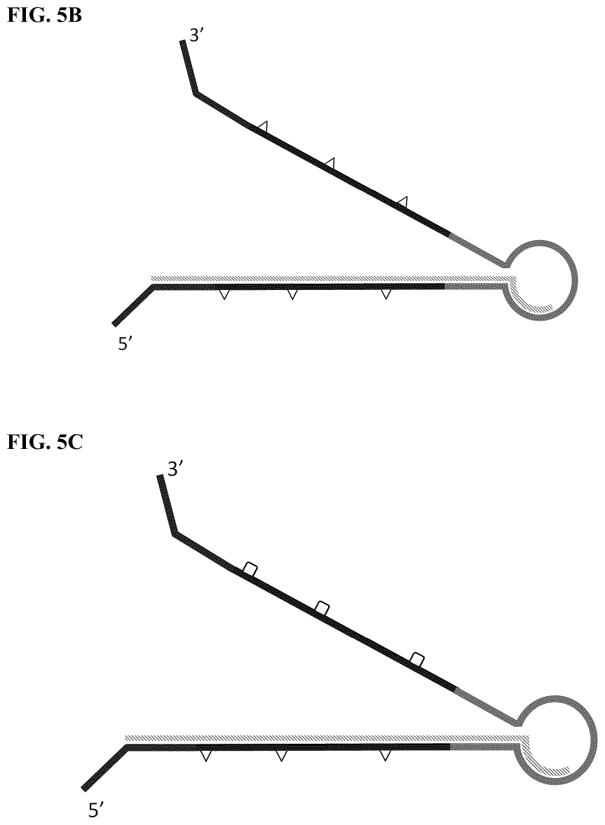

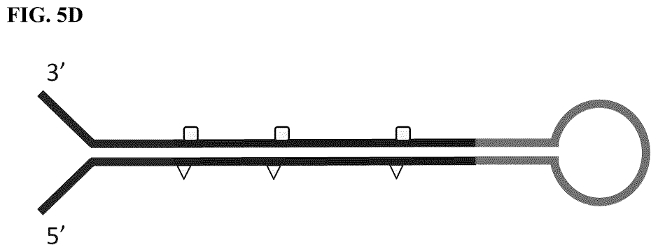

FIGS. 5A-5D illustrate an embodiment of the method described herein. FIG. 5A illustrates a nucleic acid template containing a first Y adapter, a double stranded nucleic acid, and a hairpin adapter. The double-stranded nucleic acid includes modified cytosine nucleobases, illustrated as triangles on both strands of the nucleic acid. In this embodiment, a primer anneals to the loop region of the hairpin and is extended by a polymerase (depicted as the squishy cloud) to generate a blocking strand. The blocking strand is hybridized to one of the two strands of the double-stranded nucleic acid, whereas the other strand is rendered single-stranded (FIG. 5B). Now that one of the two strands from the original dsDNA template is liberated, a conversion technique may be applied as known in the art and described herein. For example, the enzymatic and chemical conversion method depicted in FIG. 4B may be applied, which converts the modified cytosine nucleobases (depicted as triangles) to uracil nucleobase analogs (depicted as squares) as shown in FIG. 5C. Once the blocking strand is removed, the template nucleic acid may reanneal as depicted in FIG. 5D, providing a template nucleic acid with asymmetric modifications (i.e., one strand contains modified cytosine nucleobases and the other strand contains converted cytosines (e.g., uracil nucleobase analogs)).

### Example 3. Linked Duplex Sequencing: Clustering and Use in Sequencing

### Linked Duplex Sequencing: Clustering Amplification

Once formed, the library of adapter-target-adapter templates prepared according to the methods described above can be used for solid-phase nucleic acid amplification. In some embodiments of the invention, the templates used for solid-phase nucleic acid amplification have been treated with bisulfite to convert any unmethylated cytosines to uracils using protocols known in the art. In other embodiments of the invention, the templates used for solid-phase nucleic acid amplification were subjected to TET oxidation of 5mC and 5hmC to 5caC with pyridine borane reduction of 5caC to DHU, as described supra. Other suitable methods known in the art for detecting 5mC and 5hmC, including other enzymatic methods such as EM-seq and those described herein may also be applied to templates for subsequent solid-phase nucleic acid amplification.

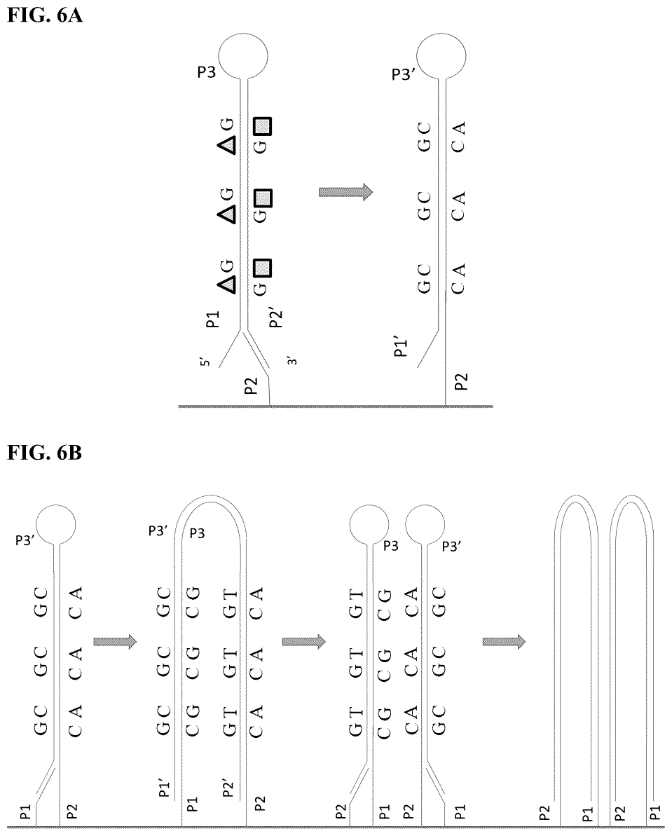

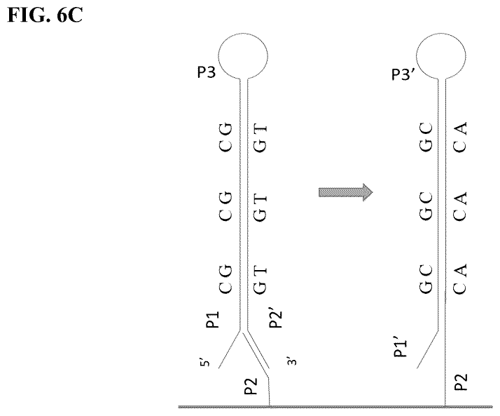

Thus, in another aspect is provided a method of nucleic acid amplification of template polynucleotide molecules which includes preparing a library of template polynucleotide molecules (e.g., adapter-target-adapter templates) and performing an amplification reaction (e.g., a solid-phase nucleic acid amplification reaction) wherein the template polynucleotide molecules are amplified. In embodiments, the method includes providing a plurality of primers (e.g., P1 and P2) that are immobilized on a solid substrate. Note, however, for clarity only a few immobilized primers are depicted in FIG. 6A.

An adapter-target-adapter construct (i.e., the denatured single strand, reading from 5′ to 3′ having the formula P1-template-P3-template-P2′ generated according to methods described herein, wherein modified cytosine nucleobases are depicted as triangles and uracil nucleobase analogs are depicted as squares) is hybridized to a complementary primer (e.g., the complement to P2′, referred to as P2) that is immobilized on a solid substrate. In the presence of a polymerase (wherein the polymerase is not shown in FIG. 6A) the P2 strand is extended to generate a complimentary copy, wherein the denatured single strand, reading from the 5′ to the 3′ has the formula P2-template-P3′-template-P1′. The original adapter-target-adapter may be removed. As shown on the right side of FIG. 6A, the complementary strand of the converted template will contain adenines that are mispaired with cytosines facilitating identification of methylation sites during sequencing analysis. Because of the self-folding of the adapter-target-adapter construct, initially seeding on the solid surface could be done without additional denaturation steps (e.g., as long as the products are in the hairpin state). In some embodiments, an amplified, cytosine-converted Y-template-hairpin construct hybridizes to an immobilized P2 primer (FIG. 6C), wherein the uracil is replaced with a thymine prior to hybridization. In the presence of a polymerase, a copy of the original template is made; this copy then hybridizes to an immobilized P1 primer as shown in FIG. 6B.

Next, the complimentary copy is annealed to a P1 primer that is immobilized on the solid substrate, which in the presence of a DNA polymerase (the polymerase is not shown in FIG. 6B) extends P1 primer to reform the original adapter-target-adapter construct (i.e., the denatured single strand having the formula P1-template-P3-template-P2′) which then hybridizes with an immobilized P2 primer. The products of the extension reaction (i.e., the P1-template-P3-template-P2′ hybridized to an immobilized P2, and P1′-template-P3′-template-P2 hybridized to P1) may be subjected to standard denaturing conditions in order to separate the extension products from strands of the adapter-target constructs. The adapter-target-adapter constructs may then anneal to a complementary immobilized primer and may be extended in the presence of a polymerase. These steps, depicted in FIGS. 6A-6B, may be repeated one or more times, through rounds of primer annealing, extension and denaturation, in order to form multiple copies of the same extension products containing adapter-target-adapter constructs, or the complements thereof. The A/C and T/G mismatches are carried forward through each round of amplification (not shown for clarity on far-right panel of FIG. 6B). Note, this bridging amplification is typically more efficient than amplifying linear strands, because the adapter-target-adapter products self-fold, thus leaving the primer site accessible.

Sequencing can be carried out using any suitable sequencing-by-synthesis technique, wherein nucleotides are added successively to a free 3′ hydroxyl group, resulting in synthesis of a polynucleotide chain in the 5′ to 3′ direction. In embodiments, the identity of the nucleotide added is determined after each nucleotide addition. In embodiments, detection of a methylated cytosine is determined by the presence of a G-T mismatch following sequencing of the amplified converted template nucleic acid. Using the methods described herein, SNPs are distinguishable from converted G-T base pairs

The term “solid-phase amplification” as used herein refers to any nucleic acid amplification reaction carried out on or in association with a solid support such that all or a portion of the amplified products are immobilized on the solid support as they are formed. The term encompasses solid-phase polymerase chain reaction (solid-phase PCR), which is a reaction analogous to standard solution phase PCR, except that both of the forward and reverse amplification primers (referred to herein as P1 and P2) are immobilized on the solid support. In practice, there will be a “plurality” of identical forward primers and/or a “plurality” of identical reverse primers immobilized on the solid support, since the PCR process requires an excess of primers to sustain amplification.

In embodiments, amplification primers for solid-phase amplification are preferably immobilized by covalent attachment to the solid support at or near the 5′ end of the primer, leaving the template-specific portion of the primer free for annealing to the cognate template and the 3′ hydroxyl group free for primer extension. Any suitable covalent attachment means known in the art may be used for this purpose. The primer itself may include a moiety, which may be a non-nucleotide chemical modification, to facilitate attachment. In embodiments, the primer may include a sulfur-containing nucleophile (e.g., phosphorothioate or thiophosphate) at the 5′ end.

In embodiments, the adapter-target-adapter templates prepared according to the methods described above can be used to prepare clustered arrays of nucleic acid colonies by solid-phase PCR amplification. The terms “cluster” and “colony” are used interchangeably herein to refer to a discrete site on a solid support comprised of a plurality of immobilized nucleic acid strands and a plurality of immobilized complementary nucleic acid strands. The term “clustered array” refers to an array formed from such clusters or colonies. In this context the term “array” is not to be understood as requiring an ordered arrangement of clusters.

### Linked Duplex Sequencing: Use in Sequencing

In another aspect is provided methods of sequencing amplified nucleic acids, optionally generated by the amplification methods described herein. The method includes optionally removing all or a portion of one immobilized strand in a “bridged” double-stranded nucleic acid structure (i.e. linearizing) and sequencing.

The products of solid-phase amplification reactions described herein wherein both P1 and P2 primers are covalently immobilized on the solid surface and may be referred to as “bridged structures” formed by annealing of pairs of immobilized polynucleotide strands and immobilized complementary strands, both strands being attached to the solid support at the 5′ end.

Arrays comprised of such bridged structures provide inefficient templates for nucleic acid sequencing, since hybridization of a conventional sequencing primer to one of the immobilized strands is not preferred compared to annealing of this strand to its immobilized complementary strand under standard conditions for hybridization. In order to provide more suitable templates for nucleic acid sequencing it is preferred to remove substantially all or at least a portion of one of the immobilized strands in the “bridged” structure in order to generate a template which is at least partially single-stranded. The portion of the template which is single-stranded will thus be available for hybridization to a sequencing primer. The process of removing all or a portion of one immobilized strand in a “bridged” double-stranded nucleic acid structure may be referred to herein as “linearization”. Bridged template structures may be linearized by cleavage of one or both strands with a restriction endonuclease or by cleavage of one strand with a nicking endonuclease. Other methods of cleavage can be used as an alternative to restriction enzymes or nicking enzymes, including chemical cleavage (e.g. cleavage of a diol linkage with periodate), cleavage of abasic sites by cleavage with endonuclease, or by exposure to heat or alkali, cleavage of ribonucleotides incorporated into amplification products otherwise comprised of deoxyribonucleotides, photochemical cleavage or cleavage of a peptide linker. Alternatively, the primers may be attached to the solid surface with a cleavable linker, such that upon exposure to a cleaving agent, all or a portion of the primer is removed from the surface.

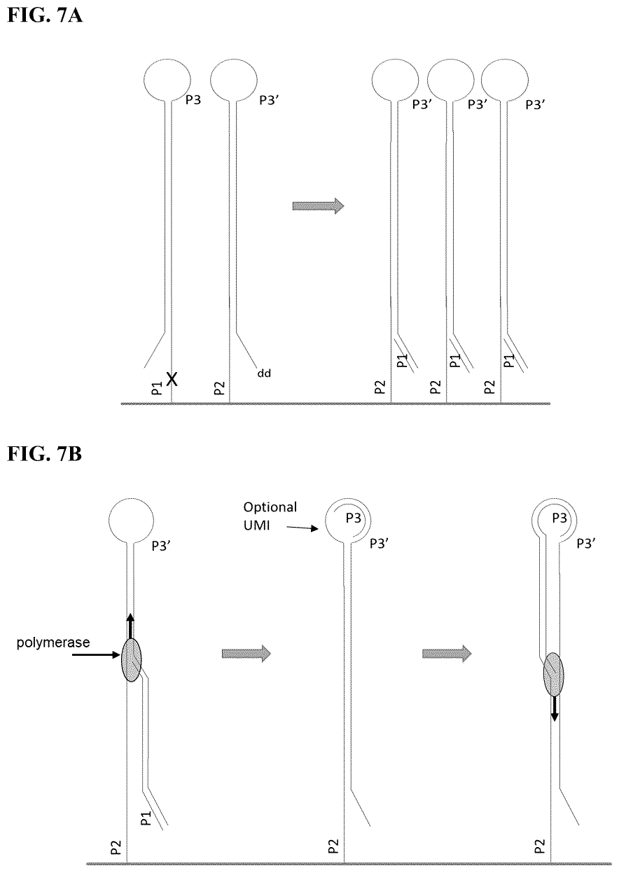

Linearization: To a solid surface having a plurality of extension products generated according to the methods described above, the method includes optionally cleaving one of the immobilized primers (e.g., P1). To the remaining extended primers (e.g., P2), the strands are terminated using dideoxy nucleotides, as shown in FIG. 7A.

Sequencing reactions: The initiation point for the first sequencing reaction is provided by annealing of a sequencing primer complementary to one of the strands in the Y adapter (e.g., P1), also shown in FIG. 7A. FIG. 7B depicts the sequencing steps. In the presence of a strand displacing polymerase, nucleotides (e.g., labeled nucleotides) are incorporated and detected such that the identity of the incorporated nucleotides allow for the identification of the first template strand. Thus, the first sequencing reaction may include hybridizing a sequencing primer to a region of a linearized amplification product, sequentially incorporating one or more nucleotides into a polynucleotide strand complementary to the region of amplified template strand to be sequenced, identifying the base present in one or more of the incorporated nucleotide(s) and thereby determining the sequence of a region of the template strand. Note, the first sequenced strand (i.e., the first primer extension product) may be i) removed; 2) terminated (e.g., introducing dideoxy nucleotides); or iii) extended and ligated to the hairpin adapter.

Next, a second sequencing reaction is initiated by annealing a sequencing primer complementary to a region in the hairpin (e.g., P3), and in the presence of a strand displacing polymerase, nucleotides (e.g., labeled nucleotides) are incorporated and detected such that the identity of the incorporated nucleotides allows for the identification of the second template strand. Thus, the second sequencing reaction may include hybridizing a sequencing primer to a region of a linearized amplification product, sequentially incorporating one or more nucleotides into a polynucleotide strand complementary to the region of amplified template strand to be sequenced, identifying the base present in one or more of the incorporated nucleotide(s) and thereby determining the sequence of a region of the template strand.

Sequencing can be carried out using any suitable sequencing-by-synthesis technique, wherein nucleotides are added successively to a free 3′ hydroxyl group, resulting in synthesis of a polynucleotide chain in the 5′ to 3′ direction. In embodiments, the identity of the nucleotide added is determined after each nucleotide addition.

In embodiments, the sequencing method relies on the use of modified nucleotides that can act as reversible reaction terminators. Once the modified nucleotide has been incorporated into the growing polynucleotide chain complementary to the region of the template being sequenced there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. Such reactions can be done in a single experiment if each of the modified nucleotides has a different label attached thereto, known to correspond to the particular base, to facilitate discrimination between the bases added at each incorporation step. Alternatively, a separate reaction may be carried out containing each of the modified nucleotides separately.

The modified nucleotides may carry a label (e.g., a fluorescent label) to facilitate their detection. Each nucleotide type may carry a different fluorescent label. However, the detectable label need not be a fluorescent label. For example, the detectable label can be a paramagnetic spin label such as nitroxide and detected by electron paramagnetic resonance and related techniques. Exemplary spin labels and techniques for their detection are described in Hubbell et al. Trends Biochem Sci. 27:288-95 (2002), which is incorporated herein by reference in its entirety. Any label can be used which allows the detection of an incorporated nucleotide. One method for detecting fluorescently labeled nucleotides includes using laser light of a wavelength specific for the labeled nucleotides, or the use of other suitable sources of illumination. The fluorescence from the label on the nucleotide may be detected by a detection apparatus (e.g., by a CCD camera or other suitable detection means).

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

### Example 4. Enzymatic-Based Approaches for Methylation Detection

As described supra, a common method of determining the methylation level and/or pattern of DNA is bisulfite conversion, a process in which genomic DNA is denatured (i.e., rendered single-stranded) and treated with sodium bisulfite, leading to deamination of unmethylated cytosine nucleobases into uracil nucleobases, while methylated cytosine nucleobases (e.g., 5-methylcytosine and 5-hydroxymethylcytosine) remain unchanged. Thus, thymine nucleobases detected in bisulfite sequencing correspond to either thymine nucleobases or unmethylated cytosine nucleobases in the original DNA, and alignment with the original template sequence easily differentiates between them. An alternative method for bisulfite-free detection of modified cytosine nucleobases (e.g., 5-methylcytosine (5mC) and 5-hydroxymethylcytosine (5hmC)) has been described (Liu Y et al. Nat. Biotechnol. 2019; 37(4): 424-429, which is incorporated herein by reference), which utilizes an enzymatic approach, and combines the ten-eleven translocation (TET) oxidation of 5mC and 5hmC to 5-carboxylcytosine (5caC) with pyridine borane reduction of 5caC to dihydrouracil (DHU). Subsequent PCR converts DHU to thymine, enabling a C-to-T transition of 5mC and 5hmC. This TET-assisted pyridine borane sequencing (TAPS) method results in higher mapping rates and more even coverage than bisulfite conversion and may be applied to the methods described herein for linked duplex methylation profiling.

Another bisulfite-free approach for methylation analysis is Enzymatic Methyl-seq (EM-Seq, described in Vaisvila R et al. bioRxiv. 2019; 12.20.884692, which is incorporated herein by reference), which uses TET2 to catalyze the oxidization of 5mC to 5hmC, 5-formulcytosine (5fC), and 5caC. T4-phage β glucosyltransferase (T4-BGT) then catalyzes the glucosylation of the formed 5hmC to 5-(β-glucosyloxymethyl)cytosine (5gmC). Combining T4-BGT with TET2 effectively protects both 5mC and 5hmC, but not cytosines, from subsequent deamination by APOBEC3A to uracils. This enzymatic manipulation of cytosine, 5mC and 5hmC enables discrimination of 5mC/5hmC from cytosine in high-throughput sequence data. 5mC and 5hmC are sequenced as cytosines, whereas unmodified cytosines are sequenced as thymines.

Two additional bisulfite-free methods for detecting cytosine modifications have been recently developed, TAPS with β-glucosyltransferase (TAPSB) and chemical-assisted pyridine borane sequencing (CAPS), allowing 5mC-specific and 5hmC-specific sequencing, respectively (for additional details, see Liu Y et al. Nature Comm. 2021; 12: 618, which is incorporated herein by reference). The TAPSB method uses BGT for selective labeling of 5hmC with glucose that enables 5hmC pulldown and protection from TET oxidation or APOBEC deamination. Following 5hmC blocking, TET oxidation and borane (e.g., pyridine borane) reduction is performed on 5mC as described in the TAPS method supra. Thereafter, 5mC are sequenced as thymines, whereas 5hmC are sequenced as cytosines. In the CAPS approach, chemical oxidation of 5hmC to 5fC is performed, which can also be converted to DHU by borane reduction. Thereafter, 5hmC are sequenced as thymines. In CAPS, the oxidation step is performed with a chemical oxidant, for example, potassium perruthenate (KRuO₄) or potassium ruthenate (K₂RuO₄). Both of these oxidants only work on single-stranded DNA. Borane reduction may be performed, for example, with 2-methylpyridine borane (pic-borane) or pyridine borane. In some embodiments, both TAPSβ and CAPS may be applied for detecting cytosine modifications to discriminate between 5mC and 5hmC modifications.

The methods described supra for enzymatic modified cytosine conversion may be applied to the sequencing workflow described in Example 2. For example, a template nucleic acid containing one or more cytosine nucleobases, wherein one or more of the cytosine nucleobases include modified cytosine nucleobases is ligated to a first adapter and a second adapter, forming a linked paired strand nucleic acid template. In some embodiments, after generating a linked paired strand template nucleic acid, the one or more cytosine nucleobases are converted to a uracil nucleobase or a uracil nucleobase analog. In some embodiments, converting the one or more cytosine nucleobases includes the TET-assisted pyridine borane sequencing (TAPS) method. In embodiments, converting the one or more cytosine nucleobases includes the Enzymatic Methyl-seq (EM-Seq) method. In some embodiments, converting the one or more cytosine nucleobases includes performing the TAPS with β-glucosyltransferase (TAPSβ). In some embodiments, converting the one or more cytosine nucleobases includes performing the chemical-assisted pyridine borane sequencing (CAPS) method.

Sequencing can be carried out using any suitable sequencing-by-synthesis technique, wherein nucleotides are added successively to a free 3′ hydroxyl group, resulting in synthesis of a polynucleotide chain in the 5′ to 3′ direction. In embodiments, the identity of the nucleotide added is determined after each nucleotide addition. In embodiments, detection of a methylated cytosine is determined by the presence of a G-T mismatch following sequencing of the amplified converted template nucleic acid. Using the methods described herein, SNPs are distinguishable from converted G-T base pairs.

### P-Embodiments

The present disclosure provides the following illustrative embodiments.

Embodiment P1. A method of sequencing a nucleic acid including one or more cytosine nucleobases, the method including: (a) ligating a first adapter to a first end of the double-stranded nucleic acid, and ligating a second adapter to a second end of the double stranded nucleic acid, wherein the second adapter is a hairpin adapter, thereby forming a nucleic acid template, wherein the nucleic acid template includes a hairpin adapter portion, a double-stranded nucleic acid portion, and a first adapter portion, wherein the double stranded nucleic acid portion includes a first template single-stranded nucleic acid sequence hybridized to a second template single-stranded nucleic acid sequence; (b) annealing a blocking primer to a sequence of the nucleic acid template and extending the blocking primer, thereby generating a blocking strand hybridized to the first template single-stranded nucleic acid sequence, displacing the second template single-stranded nucleic acid sequence, and forming a template-blocking double stranded nucleic acid portion; (c) converting the one or more cytosine nucleobases of the second template strand to a uracil nucleobase, or uracil nucleobase analog; and (d) sequencing the second template strand by annealing a sequencing primer to the nucleic acid template and extending the sequencing primer, thereby sequencing a nucleic acid including one or more cytosine nucleobases.

Embodiment P2. The method of Embodiment P1, wherein the one or more cytosine nucleobases of the second template strand include modified cytosine nucleobases.

Embodiment P3. The method of one of Embodiment P1 or Embodiment P2, wherein the modified cytosine nucleobases of the second template strand include 5-methylcytosine (5mC), 5-hydroxymethyl cytosine (5hmC), 5-formylcytosine (5fC), 5-carboxylcytosine (5caC), or β-glucosyl-5-hydroxymethylcytosine (5gmC).

Embodiment P4. The method of one of Embodiment P1 or Embodiment P2, wherein the modified cytosine nucleobases include 5-methylcytosine (5mC) or 5-hydroxymethyl cytosine (5hmC).

Embodiment P5. The method of one of Embodiment P1 or Embodiment P2, wherein converting the one or more cytosine nucleobases of the second template strand includes contacting the one or more cytosine nucleobases with a ten-eleven translocation (TET) enzyme, an apolipoprotein B mRNA editing enzyme, catalytic polypeptide-like (APOBEC) enzyme, a borane-containing reducing agent, an oxidizing agent, or a combination thereof.

Embodiment P6. The method of one of Embodiment P1 or Embodiment P2, wherein converting the one or more cytosine nucleobases of the second template strand includes i) contacting the one or more cytosine nucleobases with a ten-eleven translocation (TET) enzyme to generate one or more 5-carboxylcytosine (5caC) nucleobases; and ii) contacting the one or more 5caC nucleobases with borane-containing reducing agent to generate one or more uracil nucleobase analogs.

Embodiment P7. The method of one of Embodiment P1 or Embodiment P2, wherein converting the one or more cytosine nucleobases of the second template strand includes i) contacting the one or more cytosine nucleobases with a β-glucosyltransferase to generate one or more β-glucosyl-5-hydroxymethylcytosine (5gmC) nucleobases; ii) contacting the one or more 5gmC nucleobases with a ten-eleven translocation (TET) enzyme to generate one or more 5-carboxylcytosine (5caC) nucleobases; and iii) contacting the one or more 5caC nucleobases with borane-containing reducing agent to generate one or more uracil nucleobase analogs.

Embodiment P8. The method of one of Embodiment P1 or Embodiment P2, wherein converting the one or more cytosine nucleobases of the second template strand includes i) contacting the one or more cytosine nucleobases with an oxidizing agent to generate one or more 5-formyl cytosine (5fC) nucleobases; and ii) contacting the one or more 5caC nucleobases with borane-containing reducing agent to generate one or more uracil nucleobase analogs, wherein the oxidizing agent is selected from the group consisting of potassium perruthenate (KRuO₄), Cu(II)/TEMPO (copper(II) perchlorate and 2,2,6,6-tetramethylpiperidine-1-oxyl (TEMPO)), potassium ruthenate, and manganese oxide.

Embodiment P9. The method of one of Embodiment P1 or Embodiment P2, wherein converting one or more cytosine nucleobases of the second template strand includes i) contacting the one or more cytosine nucleobases with sodium bisulfite to generate one or more uracil nucleobases.

Embodiment P10. The method of one of Embodiment P1 or Embodiment P2, wherein converting the one or more cytosine nucleobases of the second template strand includes i) contacting the one or more cytosine nucleobases with a ten-eleven translocation (TET) enzyme to generate one or more 5-carboxylcytosine (5caC) nucleobases; and ii) contacting the second template strand with an apolipoprotein B mRNA editing enzyme, catalytic polypeptide-like (APOBEC) enzyme to generate one or more uracil nucleobases.

Embodiment P11. The method of any one of Embodiment P1 to Embodiment P10, wherein extending the blocking primer includes extending the blocking primer with a strand-displacing polymerase.

Embodiment P12. The method of any one of Embodiment P1 to Embodiment P11, further including sequencing the first template strand by annealing a second sequencing primer to the nucleic acid template and extending the sequencing primer.

Embodiment P13. The method of any one of Embodiment P1 to Embodiment P12, wherein sequencing includes sequencing by synthesis.

Embodiment P14. A method of generating a double-stranded nucleic acid including one or more cytosine mismatches, said method including: (a) ligating a first adapter to a first end of the double-stranded nucleic acid, ligating a second adapter to a second end of the double stranded nucleic acid, wherein the second adapter is a hairpin adapter, thereby forming a nucleic acid template, wherein the nucleic acid template includes a hairpin adapter portion, a double-stranded nucleic acid portion, and a first adapter portion, wherein the double stranded nucleic acid portion includes one or more cytosine nucleobases and a first template single-stranded nucleic acid sequence hybridized to a second template single-stranded nucleic acid sequence; (b) annealing a blocking primer to a sequence of the nucleic acid template and extending the blocking primer, thereby generating a blocking strand hybridized to the first template single-stranded nucleic acid sequence, displacing the second template single-stranded nucleic acid sequence, and forming a template-blocking double stranded nucleic acid portion; (c) converting the one or more cytosine nucleobases of the second template strand to a uracil nucleobase, or uracil nucleobase analog; thereby forming the double-stranded nucleic acid including one or more cytosine mismatches.

Embodiment P15. The method of any one of Embodiment P1 to Embodiment P14, wherein the blocking primer includes one or more locked nucleic acids (LNAs), 2-amino-deoxyadenosine (2-amino-dA), trimethoxystilbene-functionalized oligonucleotides (TFOs), Pyrene-functionalized oligonucleotides (PFOs), peptide nucleic acids (PNAs), or aminoethyl-phenoxazine-dC (AP-dC) nucleotides.

Embodiment P16. The method of any one of Embodiment P1 to Embodiment P15, further including amplifying the nucleic acid template including one or more cytosine mismatches to generate amplicons including one or more cytosine mismatches.

Embodiment P17. The method of any one of Embodiment P1 to Embodiment P16, further including annealing a probe oligonucleotide to the second template single-stranded nucleic acid sequence and separating the probe-hybridized double-stranded nucleic acid from nucleic acids not hybridized to a probe.

Embodiment P18. The method of any one of Embodiment P1 to Embodiment P17, wherein the first adapter is a Y-adapter.

Embodiment P19. The method of Embodiment P18, wherein the Y-adapter includes (i) a first strand having a 5′-arm and a 3′-portion, and (ii) a second strand having a 5′-portion and a 3′-arm, wherein the 3′-portion of the first strand is substantially complementary to the 5′-portion of the second strand, and the 5′-arm of the first strand is not substantially complementary to the 3′-arm of the second strand.

Embodiment P20. The method of Embodiment P19, wherein the 5′-arm of the first strand or the 3′-arm of the second strand of the Y-adapter includes a melting temperature (Tm) in a range of 60-85° C.

Embodiment P21. The method of Embodiment P19, wherein the blocking primer anneals to the 5′-portion of the second strand of the Y-adapter.

Embodiment P22. The method of any one of Embodiment P1 to Embodiment P17, wherein the first adapter is a hairpin adapter.

Embodiment P23. The method of Embodiment P22, wherein the hairpin adapter includes a 5′-end, a 5′-portion, the loop, a 3′-portion and a 3′-end, and the 5′-portion of the hairpin adapter is substantially complementary to the 3′-portion of the hairpin adapter.

Embodiment P24. The method of Embodiment P23, wherein the blocking primer anneals to a sequence within a loop of the first adapter.

Embodiment P25. The method of any one of Embodiment P1 to Embodiment P17, wherein the first adapter is a Y-adapter, and annealing a blocking primer includes: (i) hybridizing a blocking primer to a single-stranded portion of the Y-adapter, and (ii) extending the blocking primer with a strand-displacing polymerase that terminates extension within a loop of the hairpin adapter at a terminating nucleotide.

Embodiment P26. The method of any one of Embodiment P1 to Embodiment P17, wherein the first adapter is a hairpin adapter, and annealing a blocking primer includes: (i) hybridizing a blocking primer within a loop of the first hairpin adapter, and (ii) extending the blocking primer with a strand-displacing polymerase that terminates extension within a loop of the second hairpin adapter at a terminating nucleotide.

Embodiment P27. The method of any one of Embodiment P25 or Embodiment P26, wherein the terminating nucleotide includes a removable group that blocks progression of the strand-displacing polymerase, and further wherein the terminating nucleotide is treated to release the removable group prior to sequencing.

Embodiment P28. The method of any one of Embodiment P25 or Embodiment P26, wherein the terminating nucleotide is an RNA nucleotide.

Embodiment P29. The method of any one of Embodiment P1 to Embodiment P28, wherein annealing a blocking primer includes (i) forming a complex including a portion of the double-stranded nucleic acid, a blocking primer, and a homologous recombination complex including a recombinase, (ii) releasing the recombinase, and (iii) extending the blocking primer with a strand-displacing polymerase.

Embodiment P30. The method of any one of Embodiment P1 to Embodiment P28, wherein (i) annealing a blocking primer includes forming a complex including a portion of the double-stranded nucleic acid, a probe oligonucleotide, and a homologous recombination complex including a recombinase, and (ii) annealing a probe oligonucleotide to the second template single-stranded nucleic acid includes releasing the recombinase.

Embodiment P31. The method of any one of Embodiment P29 or Embodiment P30, wherein the homologous recombination complex further includes a loading factor, a single-stranded binding (SSB) protein, or both.

Embodiment P32. The method of any one of Embodiment P17 to Embodiment P31, wherein the probe oligonucleotide is covalently attached to a substrate.

Embodiment P33. The method of any one of Embodiment P17 to Embodiment P31, wherein the probe oligonucleotide is labeled with a first member of a binding pair, and separating the probe-hybridized double-stranded nucleic acid from nucleic acids not hybridized to a probe includes capturing the probe with a second member of the binding pair.

Embodiment P34. The method of Embodiment P33, wherein (i) the first member of the binding pair is biotin and the second member of the binding pair is avidin or streptavidin, or (ii) the second member of the binding pair is biotin and the first member of the binding pair is avidin or streptavidin.

Embodiment P35. The method of any one of Embodiment P1 to Embodiment P34, wherein the double-stranded nucleic acid is a cell-free DNA (cfDNA) or circulating tumor DNA (ctDNA).

Embodiment P36. A polynucleotide including, from 5′ to 3′, a first primer binding sequence, a first template single-stranded nucleic acid sequence including one or more modified cytosine nucleobases, an adapter, a second template single-stranded nucleic acid, and a second primer binding sequence, wherein the first template single-stranded nucleic acid sequence is hybridized to a blocking nucleic acid sequence; and the second template single-stranded nucleic acid includes one or more modified cytosine nucleobases.

Embodiment P37. A polynucleotide including, from 5′ to 3′, a first primer binding sequence, a first template single-stranded nucleic acid sequence, an adapter, a second template single-stranded nucleic acid, and a second primer binding sequence, wherein the second template single-stranded nucleic acid sequence is hybridized to a blocking nucleic acid sequence; and the first template single-stranded nucleic acid includes one or more modified cytosine nucleobases.

### Additional Embodiments

The present disclosure provides the following additional illustrative embodiments.

Embodiment 1. A method of sequencing a nucleic acid molecule, wherein the nucleic acid molecule comprises, from 5′ to 3′, a first strand, a first primer binding sequence, a second strand comprising a cytosine nucleobase, and a second primer binding sequence, wherein the second strand is complementary to the first strand, the method comprising: (a) annealing a blocking primer to the first primer binding sequence of the nucleic acid molecule and extending the blocking primer with a polymerase to form a blocking strand hybridized to the first strand; (b) converting the cytosine nucleobase of the second strand to a uracil nucleobase, or uracil nucleobase analog; and (c) sequencing the second strand to generate a sequencing read.

Embodiment 2. The method of Embodiment 1, wherein the cytosine nucleobase of the second strand that is converted to the uracil nucleobase, or uracil nucleobase analog, is a modified cytosine nucleobase.

Embodiment 3. The method of Embodiment 2, wherein the modified cytosine nucleobase of the second strand is a 5-methylcytosine (5mC), 5-hydroxymethyl cytosine (5hmC), 5-formylcytosine (5fC), 5-carboxylcytosine (5caC), or β-glucosyl-5-hydroxymethylcytosine (5gmC).

Embodiment 4. The method of Embodiment 2 or 3, wherein the modified cytosine nucleobase is a 5-methylcytosine (5mC) or 5-hydroxymethyl cytosine (5hmC).

Embodiment 5. The method of any one of Embodiments 1 to 4, wherein converting the cytosine nucleobase of the second strand comprises contacting the cytosine nucleobase with a ten-eleven translocation (TET) enzyme, an apolipoprotein B mRNA editing enzyme, catalytic polypeptide-like (APOBEC) enzyme, a borane-containing reducing agent, an oxidizing agent, or a combination thereof.

Embodiment 6. The method of any one of Embodiments 1 to 5, wherein converting the cytosine nucleobase of the second strand comprises i) contacting the cytosine nucleobase with a ten-eleven translocation (TET) enzyme to generate a 5-carboxylcytosine (5caC) nucleobase; and ii) contacting the 5caC nucleobase with a borane-containing reducing agent to generate a uracil nucleobase analog.

Embodiment 7. The method of any one of Embodiments 1 to 5, wherein converting the cytosine nucleobase of the second strand comprises i) contacting the cytosine nucleobase with a β-glucosyltransferase to generate a β-glucosyl-5-hydroxymethylcytosine (5gmC) nucleobase; ii) contacting the 5gmC nucleobase with a ten-eleven translocation (TET) enzyme to generate a 5-carboxylcytosine (5caC) nucleobase; and iii) contacting the 5caC nucleobase with a borane-containing reducing agent to generate a uracil nucleobase analog.

Embodiment 8. The method of any one of Embodiments 1 to 5, wherein converting the cytosine nucleobase of the second strand comprises i) contacting the cytosine nucleobase with an oxidizing agent to generate a 5-formyl cytosine (5fC) nucleobase; and ii) contacting the 5caC nucleobase with a borane-containing reducing agent to generate a uracil nucleobase analog, wherein the oxidizing agent is selected from the group consisting of potassium perruthenate (KRuO4), Cu(II)/TEMPO (copper(II) perchlorate and 2,2,6,6-tetramethylpiperidine-1-oxyl (TEMPO)), potassium ruthenate, and manganese oxide.

Embodiment 9. The method of any one of Embodiments 1 to 5, wherein converting the cytosine nucleobase of the second template strand comprises i) contacting the cytosine nucleobase with a ten-eleven translocation (TET) enzyme to generate a 5-carboxylcytosine (5caC) nucleobase; and ii) contacting the second template strand with an apolipoprotein B mRNA editing enzyme, catalytic polypeptide-like (APOBEC) enzyme to generate a uracil nucleobase.

Embodiment 10. The method of Embodiment 1, wherein the cytosine nucleobase of the second strand that is converted to the uracil nucleobase, or uracil nucleobase analog, is an unmodified cytosine nucleobase.

Embodiment 11. The method of Embodiment 10, wherein converting the unmodified cytosine nucleobase of the second strand comprises i) contacting the unmodified cytosine nucleobase with sodium bisulfite to generate a uracil nucleobase.

Embodiment 12. The method of any one of Embodiments 1 to 11, wherein the polymerase is a strand-displacing polymerase.

Embodiment 13. The method of any one of Embodiments 1 to 12, further comprising removing the blocking strand and sequencing the first strand.

Embodiment 14. The method of any one of Embodiments 1 to 13, wherein sequencing comprises sequencing by synthesis.

Embodiment 15. The method of any one of Embodiments 1 to 13, wherein sequencing comprises hybridizing a sequencing primer to the second primer binding sequence, incorporating one or more modified nucleotides into the sequencing primer with a polymerase to create an extension strand, and detecting the one or more incorporated nucleotides.

Embodiment 16. The method of any one of Embodiments 1 to 13, wherein sequencing comprises annealing a sequencing primer to the second primer binding sequence and contacting the sequencing primer with a sequencing solution comprising one or more modified nucleotides comprising a reversible terminator, and monitoring the sequential incorporation of complementary nucleotides to generate one or more sequencing reads, wherein the reversible terminator is removed prior to the introduction of the next complementary nucleotide.

Embodiment 17. A method of generating a double-stranded nucleic acid comprising a cytosine mismatch, said method comprising: (a) ligating a first hairpin adapter to a first end of the double-stranded nucleic acid molecule, and ligating a second adapter to a second end of the double-stranded nucleic acid, thereby forming a nucleic acid template, wherein the double-stranded nucleic acid comprises a first strand hybridized to a second strand, and wherein the second strand comprises a cytosine nucleobase; (b) annealing a blocking primer to a sequence of the nucleic acid template and extending the blocking primer, thereby generating a blocking strand hybridized to the first strand of the double-stranded nucleic acid, and displacing the second strand of the double-stranded nucleic acid; (c) converting the cytosine nucleobase of the displaced second strand to a uracil nucleobase, or uracil nucleobase analog, to form a converted second strand; (d) removing the blocking strand and hybridizing the first strand to the converted second strand, thereby forming the double-stranded nucleic acid comprising a cytosine mismatch.

Embodiment 18. The method of Embodiment 17, further comprising amplifying the nucleic acid template comprising the cytosine mismatch to generate amplicons comprising the cytosine mismatch.

Embodiment 19. The method of Embodiment 17 to 18, further comprising annealing a probe oligonucleotide to the displaced second strand and separating the probe-hybridized double-stranded nucleic acid from nucleic acids not hybridized to a probe.

Embodiment 20. The method of any one of Embodiments 17 to 19, wherein the second adapter is a Y-adapter.

Embodiment 21. The method of Embodiment 20, wherein the Y-adapter comprises (i) a first strand having a 5′-arm and a 3′-portion, and (ii) a second strand having a 5′-portion and a 3′-arm, wherein the 3′-portion of the first strand is substantially complementary to the 5′-portion of the second strand, and the 5′-arm of the first strand is not substantially complementary to the 3′-arm of the second strand.

Embodiment 22. The method of Embodiment 20 or 21, wherein the 5′-arm of the first strand or the 3′-arm of the second strand of the Y-adapter comprises a melting temperature (Tm) in a range of 60-85° C.

Embodiment 23. The method of Embodiment 21 or 22, wherein the blocking primer anneals to the 5′-portion of the second strand of the Y-adapter.

Embodiment 24. The method of any one of Embodiments 17 to 19, wherein the second adapter is a second hairpin adapter.

Embodiment 25. The method of Embodiment 24, wherein the second hairpin adapter comprises a 5′-end, a 5′-portion, a loop, a 3′-portion and a 3′-end, and the 5′-portion of the second hairpin adapter is substantially complementary to the 3′-portion of the second hairpin adapter.

Embodiment 26. The method of Embodiment 25, wherein the blocking primer anneals to a sequence within the loop of the second hairpin adapter.

Embodiment 27. The method of any one of Embodiments 17 to 26, wherein the blocking primer anneals to a sequence within the first hairpin adapter.

Embodiment 28. The method of any one of Embodiments 17 to 23, wherein the second adapter is a Y-adapter, and annealing a blocking primer comprises: (i) hybridizing a blocking primer to a single-stranded portion of the Y-adapter, and (ii) extending the blocking primer with a strand-displacing polymerase that terminates extension within a loop of the first hairpin adapter at a terminating nucleotide.

Embodiment 29. The method of any one of Embodiments 17 to 19 or 24 to 27, wherein the second adapter is a second hairpin adapter, and annealing a blocking primer comprises: (i) hybridizing a blocking primer within a loop of the second hairpin adapter, and (ii) extending the blocking primer with a strand-displacing polymerase that terminates extension within a loop of the first hairpin adapter at a terminating nucleotide.

Embodiment 30. The method of Embodiment 28 or 29, wherein the terminating nucleotide comprises a removable group that blocks progression of the strand-displacing polymerase, and further wherein the terminating nucleotide is treated to release the removable group prior to sequencing.

Embodiment 31. The method of Embodiment 28 or 29, wherein the terminating nucleotide is an RNA nucleotide.

Embodiment 32. The method of any one of Embodiments 17 to 27, wherein annealing the blocking primer comprises (i) forming a complex with said double-stranded nucleic acid, the blocking primer, and a homologous recombination complex comprising a recombinase, (ii) releasing the recombinase, and (iii) extending the blocking primer with a strand-displacing polymerase.

Embodiment 33. The method of Embodiment 32, wherein the homologous recombination complex further comprises a loading factor, a single-stranded binding (SSB) protein, or both.

Embodiment 34. The method of Embodiment 19, wherein the probe oligonucleotide is covalently attached to a substrate.

Embodiment 35. The method of Embodiment 19 or 34, wherein the probe oligonucleotide is labeled with a first member of a binding pair, and separating the probe-hybridized double-stranded nucleic acid from nucleic acids not hybridized to a probe comprises capturing the probe with a second member of the binding pair.

Embodiment 36. The method of Embodiment 35, wherein (i) the first member of the binding pair is biotin and the second member of the binding pair is avidin or streptavidin, or (ii) the second member of the binding pair is biotin and the first member of the binding pair is avidin or streptavidin.

Embodiment 37. The method of any one of Embodiments 17 to 36, wherein the nucleic acid molecule is a cell-free DNA (cfDNA) or circulating tumor DNA (ctDNA).

Embodiment 38. The method of any one of Embodiments 1 to 37, wherein the blocking primer comprises one or more locked nucleic acids (LNAs), 2-amino-deoxyadenosine (2-amino-dA), trimethoxystilbene-functionalized oligonucleotides (TFOs), Pyrene-functionalized oligonucleotides (PFOs), peptide nucleic acids (PNAs), or aminoethyl-phenoxazine-dC (AP-dC) nucleotides.

Embodiment 39. A polynucleotide comprising, from 5′ to 3′, a first primer binding sequence, a first template single-stranded nucleic acid sequence comprising an unmodified cytosine nucleobase, an adapter, a second template single-stranded nucleic acid, and a second primer binding sequence, wherein the first template single-stranded nucleic acid sequence is hybridized to a blocking nucleic acid sequence; and the second template single-stranded nucleic acid comprises a modified cytosine nucleobase.

Embodiment 40. A polynucleotide comprising, from 5′ to 3′, a first primer binding sequence, a first template single-stranded nucleic acid sequence, an adapter, a second template single-stranded nucleic acid, and a second primer binding sequence, wherein the second template single-stranded nucleic acid sequence is hybridized to a blocking nucleic acid sequence; and the first template single-stranded nucleic acid comprises a modified cytosine nucleobase.

1. A method of sequencing a nucleic acid molecule, wherein the nucleic acid molecule comprises, from 5′ to 3′, a first strand, a first primer binding sequence, a second strand comprising a cytosine nucleobase, and a second primer binding sequence, wherein the second strand is complementary to the first strand, the method comprising:
(a) annealing a blocking primer to the first primer binding sequence of the nucleic acid molecule and extending the blocking primer with a polymerase to form a blocking strand hybridized to the first strand;
(b) converting the cytosine nucleobase of the second strand to a uracil nucleobase, or uracil nucleobase analog; and
(c) sequencing the second strand to generate a sequencing read.

2. The method of claim 1, wherein the cytosine nucleobase of the second strand that is converted to the uracil nucleobase, or uracil nucleobase analog, is a modified cytosine nucleobase.

3. The method of claim 2, wherein the modified cytosine nucleobase of the second strand is a 5-methylcytosine (5mC), 5-hydroxymethylcytosine (5hmC), 5-formylcytosine (5fC), 5-carboxylcytosine (5caC), or β-glucosyl-5-hydroxymethylcytosine (5gmC).

4. The method of claim 3, wherein the modified cytosine nucleobase is a 5-methylcytosine (5mC) or 5-hydroxymethylcytosine (5hmC).

5. The method of claim 1, wherein converting the cytosine nucleobase of the second strand comprises contacting the cytosine nucleobase with a ten-eleven translocation (TET) enzyme, an apolipoprotein B mRNA editing enzyme, a catalytic polypeptide-like (APOBEC) enzyme, a borane-containing reducing agent, an oxidizing agent, a β-glucosyltransferase, or a combination thereof.

6. The method of claim 1, wherein converting the cytosine nucleobase of the second strand comprises i) contacting the cytosine nucleobase with a ten-eleven translocation (TET) enzyme to generate a 5-carboxylcytosine (5caC) nucleobase; and ii) contacting the 5caC nucleobase with a borane-containing reducing agent to generate a uracil nucleobase analog.

7. The method of claim 1, wherein converting the cytosine nucleobase of the second strand comprises i) contacting the cytosine nucleobase with a β-glucosyltransferase to generate a β-glucosyl-5-hydroxymethylcytosine (5gmC) nucleobase; ii) contacting the 5gmC nucleobase with a ten-eleven translocation (TET) enzyme to generate a 5-carboxylcytosine (5caC) nucleobase; and iii) contacting the 5caC nucleobase with a borane-containing reducing agent to generate a uracil nucleobase analog.

8. The method of claim 1, wherein converting the cytosine nucleobase of the second strand comprises i) contacting the cytosine nucleobase with an oxidizing agent to generate a 5-formyl cytosine (5fC) nucleobase; and ii) contacting the 5fC nucleobase with a borane-containing reducing agent to generate a uracil nucleobase analog, wherein the oxidizing agent is selected from the group consisting of potassium perruthenate (KRuO4), Cu(II)/TEMPO (copper(II) perchlorate and 2,2,6,6-tetramethylpiperidine-1-oxyl (TEMPO)), potassium ruthenate, and manganese oxide.

9. The method of claim 1, wherein converting the cytosine nucleobase of the second strand comprises i) contacting the cytosine nucleobase with a ten-eleven translocation (TET) enzyme to generate a 5-carboxylcytosine (5caC) nucleobase; and ii) contacting the second template strand with an apolipoprotein B mRNA editing enzyme, catalytic polypeptide-like (APOBEC) enzyme to generate a uracil nucleobase.

10. The method of claim 1, wherein the cytosine nucleobase of the second strand that is converted to the uracil nucleobase, or uracil nucleobase analog, is an unmodified cytosine nucleobase.

11. The method of claim 10, wherein converting the unmodified cytosine nucleobase of the second strand comprises contacting the unmodified cytosine nucleobase with sodium bisulfite to generate a uracil nucleobase.

12. The method of claim 1, wherein the polymerase is a strand-displacing polymerase.

13. The method of claim 1, further comprising removing the blocking strand and sequencing the first strand.

14. The method of claim 1, wherein sequencing comprises sequencing by synthesis.

15. The method of claim 1, wherein sequencing comprises hybridizing a sequencing primer to the second primer binding sequence, incorporating one or more modified nucleotides into the sequencing primer with a polymerase to create an extension strand, and detecting the one or more incorporated nucleotides.

16. The method of claim 1, wherein sequencing comprises annealing a sequencing primer to the second primer binding sequence and contacting the sequencing primer with a sequencing solution comprising one or more modified nucleotides comprising a reversible terminator, and monitoring the sequential incorporation of complementary nucleotides to generate one or more sequencing reads, wherein the reversible terminator is removed prior to the introduction of the next complementary nucleotide.

17.-40. (canceled)

41. The method of claim 1, wherein, prior to step (c), the nucleic acid molecule is amplified.

42. The method of claim 1, further comprising annealing a probe oligonucleotide to the displaced second strand and separating the probe-hybridized double-stranded nucleic acid from nucleic acids not hybridized to the probe.

43. The method of claim 12, wherein the extension of the blocking primer with the strand-displacing polymerase terminates at a terminating nucleotide between the second strand and the first strand.

44. The method of claim 43, wherein the terminating nucleotide comprises a removable group that blocks progression of the strand-displacing polymerase, and further wherein the terminating nucleotide is treated to release the removable group prior to sequencing.

45. The method of claim 43, wherein the terminating nucleotide is an RNA nucleotide.

46. The method of claim 42, wherein the probe oligonucleotide is covalently attached to a substrate.

47. The method of claim 42, wherein the probe oligonucleotide is labeled with a first member of a binding pair, and separating the probe-hybridized double-stranded nucleic acid from nucleic acids not hybridized to a probe comprises capturing the probe with a second member of the binding pair.

48. The method of claim 47, wherein (i) the first member of the binding pair is biotin and the second member of the binding pair is avidin or streptavidin, or (ii) the second member of the binding pair is biotin and the first member of the binding pair is avidin or streptavidin.

49. The method of claim 1, wherein the blocking primer comprises one or more locked nucleic acids (LNAs), 2-amino-deoxyadenosines (2-amino-dAs), trimethoxystilbene-functionalized oligonucleotides (TFOs), pyrene-functionalized oligonucleotides (PFOs), peptide nucleic acids (PNAs), or aminoethyl-phenoxazine-dCs (AP-dCs).
